(19) United States
(12) Patent Application Publication
(10) Pub. No.: US 2024/0032167 A1
(43) Pub. Date: Jan. 25, 2024

(54) DEVICE, METHOD AND SYSTEM FOR BIOLOGICALLY BALANCED LIGHTING

(71) Applicant: Queensland University of Technology, Queensland (AU)

(72) Inventors: Andrew J. ZELE, Queensland (AU); Beatrix FEIGL, Queensland (AU); Drew D. CARTER, Queensland (AU)

(21) Appl. No.: 18/252,172

(22) PCT Filed: Nov. 9, 2021
(86) PCT No.: PCT/AU2021/051324

§ 371 Date: May 8, 2023

(30) Foreign Application Priority Data
Nov. 9, 2020 (AU) 2020904090

(51) Int. Cl. H05B 45/20; A61M 21/00; H05B 45/325; H05B 47/11

(52) U.S. Cl. CPC H05B 45/20; A61M 21/00; H05B 45/325; H05B 47/11; A61M 2021/0044

(57) ABSTRACT

A lighting device providing emitted light with an adjustable correlated color temperature (CCT) is described. The lighting device comprises one or more light emitters and a controller configured to independently control each of the one or more light emitters. Each respective light emitter is configured to provide one or more spectral components of the emitted light, wherein the emitted light comprises a respective photoreceptor-to-photopic luminance activation ratio for each ocular photoreceptor class corresponding to an intended CCT of the emitted light. The respective photoreceptor-to-photopic luminance activation ratios comprise each of (a) a melanopsin (i)-to-photopic-luminance activation ratio; (b) a rhodopsin (R)-to-photopic-luminance activation ratio; (c) a long-wavelength sensitive opsin (L)-to-photopic-luminance activation ratio; (d) a middle-wavelength sensitive opsin (M)-to-photopic-luminance activation ratio; and (e) a short-wavelength sensitive opsin (S)-to-photopic-luminance activation ratio. Each of (a); (b); (c); (d); and (e) are within a defined range of a respective activation ratio of a blackbody radiator with the intended CCT. The emitted light may have variable excitation ratios for the five photoreceptors prompting a biological response matching that of a different blackbody radiator with changes that are invisible to the eye or difficult to perceive.

### FIELD OF THE INVENTION

The present invention relates to a device, method and system for generating biologically balanced artificial light. More particularly, this invention relates to a device, method and system for providing light with an adjustable correlated color temperature (CCT) which provides artificial light that actively modulates all ocular photoreceptor classes independently, optionally with the capability of minimising perceptual variations in color.

### BACKGROUND TO THE INVENTION

The human sleep/wake cycle is entrained to the variations in the natural solar spectrum during an approximately 24-hour day/night cycle. Natural daylight with high melanopsin photoreceptor excitation can supress the melatonin hormone to promote wakefulness and alertness. Night-time illumination conditions with high rod photoreceptor excitation can lead to an increase in the concentration of this dark (melatonin) hormone to promote sleep behaviour. Circadian rhythms are therefore influenced by a person's exposure to the quality, timing and intensity of the natural and artificial lighting spectrums in our environment.

Artificial light sources, such as typical white or red green blue (RGB) LED (Light Emitting Diode) lamps, as used in conventional buildings were developed based on the goal of enabling people to see better in a darkened environment. These conventional light sources are often built based on the combination of available technology, for example LEDs and a ‘wow’ factor, as exemplified by tuneable colored (or coloured) lights available for homes for mood lighting. This mood lighting might include, for example setting the room illumination to pink, or to warmer hues invoke a fireplace feeling.

Most current home and office lighting is not designed to produce specific biological, behavioural and/or visual responses. Artificial lights with high color temperatures, which for example may have a color temperature of daylight 6,500K, including those in computer devices, such as screens and phones, tend to have an unnaturally high melanopsin and/or rhodopsin excitation which can cause circadian disruption and negatively affect mood, sleep-wake patterns. This is a significant problem for human health and well-being.

To limit the negative effects of light on biological rhythms and mood, conventional technologies attenuate the shorter-wavelength spectral content in lights during the evening, which is good for sleep, but it has a negative effect on people during the day because shorter-wavelength visible light is the primary stimulant for synchronizing circadian rhythms and arousal. Examples include blue light filters in glasses, or selectively turning down the blue content in light displays which degrades the color representation.

Conventional and currently available LED based lighting apparatus, extending to other phosphor technologies such as, organics and laser diodes, including those with tuneable correlated color temperature (CCT) and multi-channel lighting controller units propose to better support circadian rhythms than common three-primary (red, green, blue) lighting fixtures.

Some lighting systems described in the prior art, including those specifically mentioned below, are able to exert some degree of control over circadian rhythms by tuning the color of the white light by varying the CCT. These approaches are designed with reference to the physical properties of light and its effects on vision and circadian rhythms and estimate the change in a parameter that is elicited by a change in the light to assess the effect of the change. The parameters include the Melanopsin Effectiveness Factor (MEF; see WO2016/146688A1, discussed below) and the Melanopic/Photopic Ratio (M/P; MUSCO: see WO2017/106759 and US2017/0348506 discussed below) of the light.

WO2016/146688, the publication of International Patent Application PCT/EP2016/055696 to PHILIPS LIGHTING HOLDING B.V., discloses one example of a multi-channel lighting controller unit. This document describes a three-channel lighting apparatus with the option to support the human circadian rhythm. By choosing especially the blue LED and green phosphor, the range of biological activity that can be changed is adjusted.

US2018/338359A1, the publication of U.S. patent application Ser. No. 15/875,143, to Biological Innovation & Optimization Systems, LLC, is directed to lighting systems and methods for providing biologically optimized illumination throughout the day. These systems and methods of providing LED light engines and associated illumination spectrums are described as being both visually appealing, rich in melanopic flux and reducing blue light hazard exposure. This document describes specific spectra of illumination containing high or low amounts of melanopic light, spectrally and spatially tuneable LED lighting systems, programmed and automated controllers for temporally controlling bio-effective illumination, and dimming circuitry for tuning the spectral output of lighting devices.

US2018/172227, the publication of U.S. patent application Ser. No. 15/833,023, also to Biological Innovation & Optimization Systems, LLC, is directed to light sources and methods of providing both spectrally and spatially targeted illumination using LED packages with high melanopic flux and secondary optics for spatially directing or modulating illumination to facilitate or optimize biological effects of lighting.

US2014/0104321A1, the publication of U.S. patent application Ser. No. 13/849,335, to Gary Steffy, discloses software that determines the settings and timing of luminance and color emitting from an electronic device display based on an individual's circadian rhythm preferences.

US2016/0262222, the publication of U.S. patent application Ser. No. 15/031,595 to ZUMTOBEL LIGHTING GMBH, teaches a lamp having a first light source for producing a light having a spectral distribution, wherein the light is represented by a set of chromaticity coordinates in a chromaticity diagram, and having a second light source for producing a second light having a second spectral distribution, wherein the second light is represented by a second set of coordinates in the chromaticity diagram. The control unit for controlling the light sources is designed so that an intensity of the first light can be changed independently of an intensity of the second light and the intensities are changed so that the weighting of the lights can be changed so the melanopic effect factor of the light emitted by the lamp is changed without the color temperature of the light changing.

WO2017/10675, the publication of International Patent Application No.: PCT/US2016/067340, to MUSCO CORPORATION, is directed to a method of illumination comprising comparing metamers at a known and similar CCT with at least one metamer having a higher M/P or S/P (scotopic/photopic) ratio; selecting at least one of said metamers for improved perceived brightness; evaluating the selected metamer(s) for desired CCT and acceptable CRI (Color Rendering Index); and providing light of a given CCT having increased melanopic content compared to one or more extant metameric variations of the same or similar CCT.

US2017/0348506, the publication of U.S. patent application Ser. No. 15/611,511, also to MUSCO CORPORATION, is a family member of PCT/US2016/067340, discussed above. This document is directed to improvements to circadian lighting systems based on melanopsin stimulation whereby ambient and/or device background lighting may be temporally tuned over a range of prescribed color temperatures from a first subset of lighting having a higher melanopic content to a second subset of lighting having a lower melanopic content or vice versa in accordance with a desired circadian cycle, and in a manner where net light output is of a constant perceived brightness and color throughout temporal tuning.

WO2016/199101A2, the publication of PCT/IB2016/053454 to CREE, INC., describes a device that controls a plurality of emitters through pulse width modulation (PWM) to produce a light spectrum with preferred luminous flux and efficiency, CCT, CRI, gamut and melatonin suppression characteristics, using standard CIE color metrics and reference to the dominant spectral response region of the melanopsin photopigment. Now-commonplace device control (user personalisation and temporal tuning), ambient light sensors and digital/wireless connectivity is incorporated.

U.S. Pat. No. 8,469,547 B2, to Telelumen LLC, describes a device that contains many emitters, sensors and control elements to generate a combined spectrum that mimics a preferred or recorded spectral content, with the inclusion of spatial variations. It describes the generation of broad-spectrum lighting through the use of multiple overlapping spectra and discusses the benefits of sunlight over artificially-generated light.

US 2019/0267356 A1, the publication of U.S. patent application Ser. No. 16/270,936 also to Biological Innovation and Optimization Systems LLC, describes a device that generates a spectrum intended for circadian entrainment by increasing spectral content in the effective melanopic range in conjunction with longer-wavelength light, to create a ‘white’ with increased melanopic content.

EP3422817A2, to Ind Tech Res Inst, describes a device that is capable of providing ‘white’ light with varying levels of circadian-directed spectral content (Circadian Action Factor, CAF—related to the Melanopsin Effectiveness Factor, MEF) across a range of CCTs with various CRI values. The device includes generic interface and light-emission components organised into two light groups that are switched between preferred lighting conditions.

WO2018/130403A1, the publication of PCT/EP2017/084188 to Signify Holding B.B., describes a device with a plurality of emitters organised into two light groups to generate spectra with different melanopic efficacy across a range of CCTs. The device incorporates automated adjustment of CCT and optimisation of CRI, albeit at suboptimal values.

US 2015/0062892 A1, the publication of U.S. Pat. No. 9,410,664 to SORAA Inc, describes a device with a plurality of emitters organised into two light groups that adjust the relative circadian stimulation in a defined ratio while maintaining CRI values above 80.

The reference to any prior art in this specification is not, and should not be taken as, an acknowledgement or any form of suggestion that the prior art forms part of the common general knowledge.

### SUMMARY OF THE INVENTION

Generally, the present invention is directed to a device, method and system for generating biologically balanced artificial light or electromagnetic radiation.

In a broad form, the invention relates to a device, method and system for providing light with an adjustable correlated color temperature which provides artificial light or electromagnetic radiation that actively modulates all ocular photoreceptor classes independently.

In various forms, this invention describes a device, method and system designed principally in terms of its capacity for functional control of the stimulation of all photoreceptors in the eye, and correction of those perceptual qualities (e.g. hue, saturation and/or brightness) that result from stimulation of the rod and/or melanopsin photoreceptors by the light spectrum, in order to maintain a desired cone photoreceptor response during the desired change in the melanopsin and/or rod stimulation (e.g. FIG. 10 and FIG. 11). In one embodiment, this invention describes a device, method and system designed to produce biologically directed changes in the physiological stimulation of five photoreceptor classes in the eye to produce a desired color and illumination level while accommodating, through implementation of photoreceptor-driven color corrections, a change in the cone photoreceptor stimulation to balance the visual contributions from melanopsin and/or rods to color and/or brightness perception. The device spectrums can, in line with prior art, also be operated within desired ranges specified according to metrics including, but not limited to, chromaticity, CCT and CRI and a melanopsin effectiveness factor. While incorporating similar functionalities as prior art technologies (e.g. multiple controllable output spectra, connectivity), the present invention is built upon scientific data and understanding that is not commonly known, and could not have been developed as a logical progression from the prior art without the inventors inventive contribution.

Advantageously, in another form the invention provides “biologically balanced” or “human centric lighting”. In this embodiment a device, method and system is provided for producing illumination specific to all ocular photoreceptor classes, melanopsin containing intrinsically photosensitive Retinal Ganglion Cells (ipRGC; or (i)); rods (R); and the three cone classes L-, M- and S-. Advantageously, the invention may positively benefit behaviour and/or will not have the disadvantages of existing illumination methods and devices which disrupt circadian rhythm or negatively affect mood or sleep-wake patterns. Further advantages may include one or more of promotion of wakefulness, increased autonomic nervous system arousal and improved sleep. By improving sleep quality, the invention may aid productivity, health and well-being and quality of life. As will be elucidated below another possible advantage is increased energy efficiency.

In a first aspect, although it need not be the only or indeed the broadest aspect, the invention provides a lighting device providing emitted light with an adjustable correlated color temperature (CCT), the lighting device comprising:
one or more light emitters wherein each respective light emitter is configured to provide one or more spectral components of the emitted light, wherein the emitted light comprises a respective photoreceptor-to-photopic luminance activation ratio for each ocular photoreceptor class corresponding to an intended CCT of the emitted light, the respective photoreceptor-to-photopic luminance activation ratios comprising:
(a) a melanopsin (i)-to-photopic-luminance activation ratio within a defined range of a melanopsin-to-photopic-luminance activation ratio of a blackbody radiator with the intended CCT;
(b) a rhodopsin (R)-to-photopic-luminance activation ratio within a defined range of a rhodopsin-to-photopic-luminance activation ratio of a blackbody radiator with the intended CCT;
(c) a long-wavelength sensitive opsin (L)-to-photopic-luminance activation ratio within a defined range of a long-wavelength sensitive opsin-to-photopic-luminance activation ratio of a blackbody radiator with the intended CCT;
(d) a middle-wavelength sensitive opsin (M)-to-photopic-luminance activation ratio within a defined range of a middle-wavelength sensitive opsin-to-photopic-luminance activation ratio of a blackbody radiator with the intended CCT; and
(e) a short-wavelength sensitive opsin (S)-to-photopic-luminance activation ratio within a defined range of a short-wavelength sensitive opsin-to-photopic-luminance activation ratio of a blackbody radiator with the intended CCT; and
a light emitter controller, configured to independently control each of the one or more light emitters to provide the emitted light.

In a second aspect, the invention provides a method for providing emitted light with an adjustable correlated color temperature, the method comprising:
independently controlling one or more light emitters to provide emitted light wherein each respective light emitter is configured to provide one or more spectral components of the emitted light, wherein the emitted light comprises a respective photoreceptor-to-photopic luminance activation ratio for each ocular photoreceptor class corresponding to an intended CCT of the emitted light, the respective photoreceptor-to-photopic luminance activation ratios comprising:
(a) a melanopsin (i)-to-photopic-luminance activation ratio within a defined range of a melanopsin-to-photopic-luminance activation ratio of a blackbody radiator with the intended CCT;
(b) a rhodopsin (R)-to-photopic-luminance activation ratio within a defined range of a rhodopsin-to-photopic-luminance activation ratio of a blackbody radiator with the intended CCT;
(c) a long-wavelength sensitive opsin (L)-to-photopic-luminance activation ratio within a defined range of a long-wavelength sensitive opsin-to-photopic-luminance activation ratio of a blackbody radiator with the intended CCT;
(d) a middle-wavelength sensitive opsin (M)-to-photopic-luminance activation ratio within a defined range of a middle-wavelength sensitive opsin-to-photopic-luminance activation ratio of a blackbody radiator with the intended CCT; and
(e) a short-wavelength sensitive opsin (S)-to-photopic-luminance activation ratio within a defined range of a short-wavelength sensitive opsin-to-photopic-luminance activation ratio of a blackbody radiator with the intended CCT.

In a third aspect, the invention provides a system for providing emitted light with an adjustable correlated color temperature, the system comprising:
one or more light emitters wherein each respective light emitter is configured to provide one or more spectral components of the emitted light, wherein the emitted light comprises a respective photoreceptor-to-photopic luminance activation ratio for each ocular photoreceptor class corresponding to an intended CCT of the emitted light, the respective photoreceptor-to-photopic luminance activation ratios comprising:
(a) a melanopsin (i)-to-photopic-luminance activation ratio within a defined range of a melanopsin-to-photopic-luminance activation ratio of a blackbody radiator with the intended CCT;
(b) a rhodopsin (R)-to-photopic-luminance activation ratio within a defined range of a rhodopsin-to-photopic-luminance activation ratio of a blackbody radiator with the intended CCT;
(c) a long-wavelength sensitive opsin (L)-to-photopic-luminance activation ratio within a defined range of a long-wavelength sensitive opsin-to-photopic-luminance activation ratio of a blackbody radiator with the intended CCT;
(d) a middle-wavelength sensitive opsin (M)-to-photopic-luminance activation ratio within a defined range of a middle-wavelength sensitive opsin-to-photopic-luminance activation ratio of a blackbody radiator with the intended CCT; and
(e) a short-wavelength sensitive opsin (S)-to-photopic-luminance activation ratio within a defined range of a short-wavelength sensitive opsin-to-photopic-luminance activation ratio of a blackbody radiator with the intended CCT; and
a light emitter controller, configured to independently control each of the one or more light emitters to provide the emitted light.

In a fourth aspect, the invention provides a computer program product comprising a non-transitory computer usable medium comprising:
a computer usable medium and computer readable program code embodied on said computer usable medium, the code for providing emitted light with an adjustable correlated color temperature (CCT), the computer readable code comprising:
computer readable program code devices (1) configured to cause the computer to control one or more light emitters wherein each respective light emitter is configured to provide one or more spectral components of the emitted light, wherein the emitted light comprises a respective photoreceptor-to-photopic luminance activation ratio for each ocular photoreceptor class corresponding to an intended CCT of the emitted light, the respective photoreceptor-to-photopic luminance activation ratios comprising:
(a) a melanopsin (i)-to-photopic-luminance activation ratio within a defined range of a melanopsin-to-photopic-luminance activation ratio of a blackbody radiator with the intended CCT;
(b) a rhodopsin (R)-to-photopic-luminance activation ratio within a defined range of a rhodopsin-to-photopic-luminance activation ratio of a blackbody radiator with the intended CCT;
(c) a long-wavelength sensitive opsin (L)-to-photopic-luminance activation ratio within a defined range of a long-wavelength sensitive opsin-to-photopic-luminance activation ratio of a blackbody radiator with the intended CCT;
(d) a middle-wavelength sensitive opsin (M)-to-photopic-luminance activation ratio within a defined range of a middle-wavelength sensitive opsin-to-photopic-luminance activation ratio of a blackbody radiator with the intended CCT; and
(e) a short-wavelength sensitive opsin (S)-to-photopic-luminance activation ratio within a defined range of a short-wavelength sensitive opsin-to-photopic-luminance activation ratio of a blackbody radiator with the intended CCT;
and
computer readable program code devices (2) configured to cause the computer to independently control each of the one or more light emitters to provide the emitted light.

In further aspects, the invention provides a method of photoentrainment; a method of treating an ophthalmic disease, disorder or condition; a method of treating a neurologic disease, disorder or condition; a method of treating metabolic disease, disorder or conditions; a method of treating a sleep disease, disorder or condition; a method of treating a mood disease, disorder or condition; a method of treating a circadian rhythm disease, disorder or condition; a method of promoting and/or aiding sleep or wakefulness and/or alertness; a method of supporting biorhythm; or a method of conserving energy during illumination comprising providing emitted light with an adjustable correlated color temperature (CCT), comprising:
independently controlling one or more light emitters to provide emitted light wherein each respective light emitter is configured to provide one or more spectral components of the emitted light, wherein the emitted light comprises a respective photoreceptor-to-photopic luminance activation ratio for each ocular photoreceptor class corresponding to an intended CCT of the emitted light, the respective photoreceptor-to-photopic luminance activation ratios comprising:
(a) a melanopsin (i)-to-photopic-luminance activation ratio within a defined range of a melanopsin-to-photopic-luminance activation ratio of a blackbody radiator with the intended CCT;
(b) a rhodopsin (R)-to-photopic-luminance activation ratio within a defined range of a rhodopsin-to-photopic-luminance activation ratio of a blackbody radiator with the intended CCT;
(c) a long-wavelength sensitive opsin (L)-to-photopic-luminance activation ratio within a defined range of a long-wavelength sensitive opsin-to-photopic-luminance activation ratio of a blackbody radiator with the intended CCT;
(d) a middle-wavelength sensitive opsin (M)-to-photopic-luminance activation ratio within a defined range of a middle-wavelength sensitive opsin-to-photopic-luminance activation ratio of a blackbody radiator with the intended CCT; and
(e) a short-wavelength sensitive opsin (S)-to-photopic-luminance activation ratio within a defined range of a short-wavelength sensitive opsin-to-photopic-luminance activation ratio of a blackbody radiator with the intended CCT.

In still further aspects the device or system of the first or third aspects may be used or when used in one or more of the methods of the further aspects.

According to one embodiment one of the above aspects, each respective light emitter of the one or more light emitters may comprise one or more radiation emitters such as one or more phosphor. The one or more radiation emitters may be arranged in one or more array, bank or group. Each array, bank or group, or a subset of the one or more emitters comprised in each array, bank or group, may be independently controlled.

According to still another embodiment of any one of the above aspects, the one or more light emitters may comprise five light emitters, wherein each of the five light emitters emit light comprising one spectral component comprising light eliciting the photoreceptor-to-photopic luminance activation ratio for a respective one of (a) i; (b) R; (c) L; (d) M; or (e) S.

According to yet another embodiment of any one of the above aspects, at least one of the one or more light emitters emit light comprising two or more spectral components wherein each spectral component comprises light eliciting the photoreceptor-to-photopic luminance activation ratio for a respective one of (a) i; (b) R; (c) L; (d) M; or (e) S.

According to another embodiment of any one of the above aspects, the emitted light produces a photoreceptor class-to-photopic-luminance activation defined by a respective cubic polynomial which is a function of CCT for each respective ocular photoreceptor class: i; R; L; M; and S. The photoreceptor class-to-photopic-luminance activation ratios may be defined as being unit-normalized to equal-luminance. The photoreceptor class-to-photopic-luminance activation ratios may be defined using one or more sensitivity function. In one embodiment, each respective cubic polynomial for each ocular photoreceptor class i; R; L; M; and S may comprise ax³+bx²+cx+d where x is a desired CCT divided by 1,000. The emitted light may comprise each of the one or more spectral components.

In another embodiment according to any one of the above aspects, the defined range for each respective ocular photoreceptor class comprises an increase and/or decrease relative to a target activation ratio of each respective photoreceptor class: i; R; L; M; and S.

In still another embodiment of any one of the above aspects, the intended perceived color is maintained while melanopsin (i) stimulation and/or rhodopsin (R) stimulation may be varied to simulate an effect of the sun at the same or a different CCT. The perceived color may be maintained by correcting for any color contributions from melanopsin (i) stimulation and/or rhodopsin (R) stimulation.

In one embodiment, the defined range may be defined by:
ax³+bx²+cx+d±B wherein the value for B for each respective ocular photoreceptor class i; R; L; M; and S comprises 0.01 to 0.1; 0.02 to 0.08; or 0.025 to 0.075.

In another embodiment, the defined range may be defined by:
ax³+bx²+(c±(B/F))x+d wherein the value for the bound for each respective ocular photoreceptor class comprises 0.01 to 0.1; 0.02 to 0.08; or 0.025 to 0.075, wherein B is between 3.0 and 10.0 wherein F may be selected from 3.0; 3.5; 4.0; 4.5; 5.0; 5.5; 6.0; 6.5; 7.0; 7.5; 8.0; 8.5; 9.0; 9.5 or 10.0. In a particular embodiment, B is 4.0 or 6.5. In another particular embodiment, the defined range may apply for a CCT greater than or equal to 3,000K. In another particular embodiment, the value for B for i=0.05; R=0.05; for L=0.025, for M=0.025; and for S=0.075.

In another embodiment according to any one of the above aspects, for the melanopsin-to-photopic-luminance ratio the coefficient may be 0.001953407, the coefficient b may be −0.047973588, the coefficient c may be 0.461696814, the coefficient d may be −0.412592279, and B may be 0.05.

In one embodiment according to any one of the above aspects, for the rhodopsin-to-photopic-luminance activation ratio according to any one of the above aspects, the coefficient a may be 0.002189895, the coefficient b may be −0.049925408, the coefficient c may be 0.435812967, the coefficient d may be −0.244240435, and B may be 0.05.

In another embodiment according to any one of the above aspects, for the long-wavelength-sensitive-opsin-to-photopic-luminance ratio the coefficient may be −0.000505513, the coefficient b may be 0.010270342, the coefficient c may be −0.074854481, the coefficient d may be 0.846873597, and B may be 0.025.

In still another embodiment according to any one of the above aspects, for the medium-wavelength-sensitive-opsin-to-photopic-luminance ratio the coefficient a may be the coefficient b may be −0.010270342, the coefficient c may be 0.074854481, the coefficient d may be 0.153126403, and B may be 0.025.

In yet another embodiment according to any one of the above aspects, for the short-wavelength-sensitive-opsin-to-photopic-luminance ratio the coefficient a may be the coefficient b may be −0.034754594, the coefficient c may be 0.433092008, the coefficient d may be −0.619542968, and B may be 0.075.

In still another embodiment according to any one of the above aspects, the coefficient values for each respective ocular photoreceptor class may be as shown in Table 1.

In yet another embodiment according to any one of the above aspects, the adjustable CCT comprises an adjustment from a first CCT as defined by x or as shown in Table 2 to a second different CCT as defined by x or as shown in Table 2. The adjustment may be within 2,000 to 8,000K; 3,000 to 8,000K; 3,200 to 8,000K; 3,500 to 8,000K; or 4,000 to 8,000K.

According to any one of the above aspects, the emitted light may be referenced to the 10 degree CIE (International Commission on Illumination) 1964 Colorimetric Observer.

According to yet another embodiment of any one of the above aspects, the respective ocular photoreceptor activation ratio may be independent of the illumination level of the emitted light.

According to still another embodiment of any one of the above aspects, the emitted light may comprise a CRI of 80 or higher; 85 or higher; 90 or higher; 91 or higher; 92 or higher; 93 or higher; 94 or higher; 95 or higher; 96 or higher; 97 or higher; 98 or higher; 99 or higher.

According to any one of the above aspects, component photoreceptor-to-photopic luminance activation spectra ratios may be specified with reference to a 12-bit scale.

According to any one of the above aspects, active stimulation of all ocular photoreceptor classes may be directly due to the emitted electromagnetic radiation that is emitted from the one or more emitters. The active stimulation of all ocular photoreceptor classes may be in a defined ratio. The defined ratio may be to a stimulation correlated to a part of a solar day. The stimulation correlated to a part of a solar day may be in synchronisation with the solar day at that location or time-shifted from the solar day at that location. The defined ratio may be to a stimulation correlated to one or more dysfunctional photoreceptor class in the retina due to, for example an ophthalmic disease or condition. The stimulation correlated to the one or more dysfunctional photoreceptor class may comprise increasing the dysfunctional photoreceptor activation to a normal or functional level.

According to any one of the above aspects, the one or more emitter may emit light or electromagnetic radiation comprising a bandwidth between 420 and 650 nm or between 300 and 780 nm. The emitted light or electromagnetic radiation may comprise at least five distinctly controllable spectral components, which may comprise spectral components at 420 to 470 nm; 460 to 510 nm; 500 to 550 nm; 540 to 600 nm; and 580 to 650 nm. The emitted light or electromagnetic radiation may comprise a sixth distinctly controllable spectral component at 500 to 610 nm. In a particular embodiment, the emitted light electromagnetic radiation may comprise eight unique spectral components. The eight unique spectral components, defined by peak wavelength and deviation from peak wavelength at half-maximum, may comprise: 440±5 nm; 459±5 nm; 473±5 nm; 499±524±5 nm; 567±5 nm; 592±8 nm; and 632±8 nm.

According to any one of the above aspects, the one or more emitter comprises one or more broadband emitter. The one or more broadband emitter may comprise a white LED. At least one emitter of the one or more broadband emitter may emit a blue-shifted yellow electromagnetic radiation.

According to any one of the above aspects, the one or more emitter may comprise a plurality of emitters, wherein each of the plurality of emitters emits light or electromagnetic radiation at one or more bandwidth between 420 and 650 nm or between 300 and 780 nm. The plurality of emitters may comprise distinctly controllable emitters emitting light or electromagnetic radiation at the following spectral components: 420 to 470 nm; 460 to 510 nm; 500 to 550 nm; 540 to 600 nm; and 580 to 650 nm. The distinctly controllable emitters may further emit light or electromagnetic radiation at the spectral component: 500 to 610 nm. The distinctly controllable emitters may emit light or electromagnetic radiation at the following spectral components, defined by peak wavelength and deviation from peak wavelength at half-maximum: 440±5 nm; 459±5 nm; 473±5 nm; 499±5 nm; 524±5 nm; 567±5 nm; 592±8 nm; and 632±8 nm.

The plurality of emitters may comprise a respective emitter for each spectral component. The plurality of controllable emitters may comprise a fewer number of emitters than spectral components wherein at least one of the controllable emitters emits light or electromagnetic radiation at two or more of the spectral components. The plurality of controllable emitters may comprise a larger number of emitters than spectral components wherein at least two of the controllable emitters emits light or electromagnetic radiation to create one spectral component.

According to any one of the above aspects, five or more channels of light or electromagnetic radiation emission are provided. The five or more channels may comprise respective channels to the distinct spectral components. In a particular embodiment eight channels are provided.

Each distinct spectral component may be associated with light or electromagnetic radiation emitted by a respective emitter. Each distinct spectral component may comprise a subset of the electromagnetic radiation spectrum. The subset of the electromagnetic radiation spectrum may be a discrete continuum of the electromagnetic spectrum.

The active stimulation of all ocular photoreceptor classes by the artificial light or electromagnetic radiation may be independent active stimulation. The independent active stimulation may comprise one or a combination of multiple respective channels and/or spectral components of emitted light or electromagnetic radiation for dominant influence of one or more respective ocular photoreceptor class. The active stimulation may map daylight; daytime; dusk; dawn; and/or night-time photoreceptor excitations with variation in CCT.

The one or more emitter may comprise one; two; three; four; five; six; seven; eight; nine; ten; or ten or more emitters.

According to any one of the above aspects the controllable spectral components may comprise narrowband or one or more wideband output including but not limited to: 1 nm; 2 nm; 3 nm; 4 nm; 5 nm; 6 nm; 7 nm; 8 nm; 9 nm; 10 nm; 15 nm; 20 nm; 25 nm; 30 nm; 40 nm; 45 nm; 50 nm; 60 nm; 70 nm; 80 nm; 90 nm; 100 nm; or 110 nm.

According to any one of the above aspects, each emitter may comprise one or more light source such as, an LED; one or more biologically luminescent material; and/or stimulated emission for example, from a LASER (light amplification by stimulated emission of radiation). Each emitter may comprise one or more of a solid state light source; an organic luminescent material and/or inorganic luminescent material. Each emitter may comprise a spectrum bandwidth that may either be un-attenuated, or optically attenuated using organic or inorganic substrates to narrow their output spectrum. The one or more emitter may be provided in a combination to control the available modulation ratios of the five classes of human photoreceptors.

In one embodiment of any one of the above aspects, each of the one or more emitter may further comprise one or more filter. The one or more filter may comprise one or more colored interference filter; one or more spectral filter; one or more neutral density filter for tuning the emitted light electromagnetic radiation. The emitted light electromagnetic radiation may comprise white light.

According to any one of the above aspects, the one or more emitter, or respective emitters may be independently controllable by the light emitter controller. The independent control may be for spectral power manipulation corresponding to a set of photoreceptor class ratios.

According to any one of the above aspects, an absolute illumination level may be modulated in any desired set of photoreceptor ratios (note: dim lighting is an important stimulus for circadian photoentrainment). The one or more emitters integrated in single or multiple emitters enable dimming (output level decrease) while providing the same photoreceptor ratios independent of the illumination level (output level).

According to any one of the above aspects, the light emitter controller may comprise one or more computer processor. The controller, computer or computer processor may comprise one or more of a microcontroller; a Field-programmable Gate Array (FPGA); or other control generating device. The controller, computer or computer processor may be comprised in a single system or distributed systems.

According to any one of the above aspects, the light emitter controller may utilise a minimisation algorithm (optimisation) to compute spectral outputs based on individual photoreceptor responses. The algorithm may produce high Color Rendering Index (CRI) solutions for all CCTs through the daylight spectrum with variations in Melanopsin and/or Rhodopsin stimulation.

According to any one of the above aspects, the light emitter controller may utilise pulsed width modulation (PWM) dimming and/or illumination source measurement.

According to any one of the above aspects, one or more sensor may be comprised for detecting light or electromagnetic radiation. The detected light or electromagnetic radiation may comprise the emitted light or electromagnetic radiation. The detected light or electromagnetic radiation may be analysed. The analysis may comprise a spectral analysis. The analysis may be in real-time or close to real-time.

According to any one of the above aspects, the light emitter controller may comprise one or more processor to determine one or more control value for a desired light or electromagnetic radiation emission and may control the illumination source to modulate the emitted light or electromagnetic radiation to the desired emitted light or electromagnetic radiation. The desired emitted light or electromagnetic radiation emission may comprise an artificial spectrum or a natural spectrum. The natural spectrum may comprise a solar spectrum. The desired emitted light electromagnetic radiation may be to one or more control value for each emitter over time. The one or more control value may be for a desired photoreceptor response(s) which may optionally vary over time. The one or more control value may correct for perceptual color variations caused by color contributions of ipRGCs (melanopsin (i)) and/or rods (rhodopsin (R)).

According to any one of the above aspects the device may generate emitted light comprising a spectrum from a desired set of photoreceptor ratios. The desired set of photoreceptor ratios may comprise (a) a melanopsin (i)-to-photopic-luminance activation ratio and/or (b) a rhodopsin (R)-to-photopic-luminance activation ratio calculated from a natural spectrum and (c) a long-wavelength sensitive opsin (L)-to-photopic-luminance activation ratio; (d) a middle-wavelength sensitive opsin (M)-to-photopic-luminance activation ratio; and (e) a short-wavelength sensitive opsin (S)-to-photopic-luminance activation ratio chosen for a given application or preference to generate a perceived color. The perceived color may be selected from a variety of colors such as, blue, green, yellow, orange, purple, pink or red for example. For an absolute Photopic luminance, a natural spectrum may be defined by any one of the above aspects.

According to any one of the above aspects, the sensor may measure ambient illumination. The device may utilise one or more spectra of daily light exposure, in order to calculate and provide supplemental light exposure.

The minimization algorithm may optimize the component spectra contributions to decrease the area under the curve (AUC) of the emitted light spectra (power).

According to any one of the above aspects, the light emitter controller may control the one or more emitter to dynamically control melanopsin (i) and rhodopsin (R) photoreceptor activation. The light emitter controller may allow any realisable contrast level (flux) relative to constant (or variable) melanopsin (i), rhodopsin (R) and/or cone-opsin (L-; M-; S-) activation. The control may comprise real-time or near real-time modulation.

According to any one of the above aspects, the emitted light or electromagnetic radiation may comprise a light spectrum that produces photoreceptor activations that are closer to that resultant from the environmental broadband solar spectrum.

According to any one of the above aspects, the one or more emitter may comprise a different primary combination to create a white light based on the CCT. The illumination may provide a constant CCT while varying melanopsin (i) and/or rhodopsin (R) activation. Advantageously, this may correct for changes in color appearance caused by variations in melanopsin (i) and rhodopsin (R) activation.

According to any one of the above aspects, the CRI may be higher than conventional white LEDs. The higher CRI may be due to a more uniform spectral output. The CRI may comprise a higher Color Rendering Index (CRI) than the Bio Hue Lamp. The CRI may be varied by adjusting the spectral composition of the illumination source. The CRI may be varied by adjusting the bandwidth and/or dominant wavelength and/or spectral distribution of the one or more emitter.

According to any one of the above aspects, the emitted light or electromagnetic radiation stimulates all photoreceptors comprising melanopsin containing intrinsically photosensitive retinal ganglion cells; rods; and cones optionally at a system or user defined ratio. The emitted light or electromagnetic radiation may stimulate all ocular photoreceptor proteins comprising: melanopsin; rhodopsin; and opsins. The opsins may comprise three opsins. The three opsins may comprise long wavelength sensitive opsin (erythrolabe) or red opsin; middle wavelength sensitive opsin (chlorolabe) or green opsin; and short wavelength sensitive opsin (cyanolabe) or blue opsin. The stimulation may independently control the activity level of each of the photoreceptors.

According to any one of the above aspects, a Color Rendering Index (CRI) closer to natural lighting (CRI=100) than prior art devices, methods and system may be provided.

According to any one of the above aspects, to mimic the effects of the sun on the photoreceptor excitations, with variation in CCT during the solar day, the following photoreceptor Weber contrast changes, relative to 5,500K, may be required:
Melanopsin contrasts: −33% at low CCT to +18% at high CCT; and
Rod contrasts: −27% at low CCT to +14% at high CCT.

According to any one of the above aspects, one or more biological effects may be prompted by the emitted light such as, photoreceptor excitations influencing a circadian rhythm aligned to changes in the solar day by transitioning the device spectrum from low melanopsin (i) and rhodopsin (R) excitations in the morning (e.g. for a low blackbody CCT), to higher melanopsin (i) and rhodopsin (R) excitations during the day, to low values in the evening. The parameters prompting these biological effects may be aligned to seasonal variation and/or geographical location.

According to another embodiment of any one of the above aspects, the emitted light comprises a spectrum that prompts a set of melanopsin (i) and rhodopsin (R) excitations that may match an alternative time of day to those elicited by the current natural solar day in the geographical location and time of day of the user with minimal perceptual variation. These spectrums may be set to a desired circadian entrainment pattern, an individual sleep/wake and/or alertness preferences resulting from, for example, occupational requirements (e.g. shift-work) or travel (e.g. jet-lag).

According to yet another embodiment of any one of the above aspects, the emitted light generates perceptually invariant changes in the melanopsin (i) and rhodopsin (R) excitations that may be higher and/or lower than a fixed blackbody radiator by combining melanopsin (i) and rhodopsin (R) excitations for the desired circadian effect with the cone excitations of the preferred blackbody radiator and subsequent corrections to account for color variation.

According to any one of the above aspects, the emitted light may provide a precise manipulation of visual perception and biological rhythms linked to industry defined human standard observer functions.

According to any one of the above aspects, the emitted light may provide a broader band spectral distribution to provide a truer representation of the natural environment may be provided.

In one embodiment of any one of the above aspects, the emitted light may not be perceived differently from ambient light (chromaticity invariant) and/or may be directed to circadian rhythm.

In another embodiment of any one of the above aspects, the emitted light may be modulated with circadian rhythm. The modulation may mimic the natural variation in a solar day such as, cooler (i.e. bluer) during the day and warmer (i.e. more orangish) during the evening. The modulation may be to mimic changes in environmental illumination. The modulation may mimic the natural variation in photoreceptor activations that change during a solar day, while maintaining a single nominated color appearance. The modulation may be different to the natural circadian rhythm. The modulation may be to prepare for travel to a different time zone; to recover from travel from a different time zone or jetlag; to synchronize with work or other activity. The modulation may restore dysfunctional photoreceptor(s) activity in a person with ophthalmic disease to normal (functional) levels.

According to any one of the above aspects, the emitted light may be for ambient lighting. The ambient lighting may be provided at a home; at a workplace; at a school; at a childcare centre; at a hospital; at a nursing home; at a hotel; at a sleeping quarters; in a transit vehicle; at a road; at a sports field or any site of human activity including sleep.

The device, method, system and computer program product according to any one of the above aspects may be comprised in one or more electronic device such as, a visual display unit, a computer device or illuminated billboard. The ambient lighting may be provided at a site of a visual display such as, a museum or gallery.

The device or system according to any one of the above aspects may comprise a luminaire.

According to any one of the above aspects, emitted light and associated stimulation or perception may be an animals' stimulation or perception. The animal may be a human. In other embodiments, the animal may be a companion animal; a performance animal; or another animal.

According to any one of the above aspects, the invention can be retrofitted to existing multi-spectrum light sources.

According to any one of the above aspects, the intended CCT in a first room or zone may be different to the intended CCT in a second, different room or zone. The first room or zone may comprise a first device or system according to the above aspects and the second room or zone may comprise a second device or system according to the above aspects. The intended perceived color may be constant or substantially constant in both the first room or zone and the second, different room or zone while melanopsin (i) stimulation and/or rhodopsin (R) stimulation may be varied to simulate an effect of the sun at the same or a different CCT. The perceived color may be maintained by correcting for any color contributions from melanopsin (i) stimulation and/or rhodopsin (R) stimulation.

According to any one of the above aspects, the one or more emitter may be comprised in an illumination source.

According to any of the above aspects, control may be via a wireless communication protocols.

Further aspects and/or features of the present invention will become apparent from the following detailed description.

### BRIEF DESCRIPTION OF THE DRAWINGS

In order that the invention may be readily understood and put into practical effect, reference will now be made to embodiments of the present invention with reference to the accompanying drawings, wherein like reference numbers refer to identical elements. The drawings are provided by way of example only, wherein:

FIG. 1A: shows component light spectra emitted from a device according to one embodiment of the invention.

FIG. 1B: shows another component light spectra emitted from a device according to another embodiment of the invention.

FIG. 2A: is a photoreceptor-directed spectra for altering the melanopsin and rhodopsin photoreceptor excitation based on the spectrum of FIG. 1A. Key: Solid line: Balanced spectrum (Reference). Black dashed line: Melanopsin increase. Grey dashed line: Melanopsin decrease. Black dotted line: Rhodopsin increase. Grey dotted line: Rhodopsin decrease. Black dash-dot line: Melanopsin and rhodopsin increase. Grey dash-dot line: Melanopsin and rhodopsin decrease.

FIG. 2B: is a photoreceptor-directed spectra for altering the melanopsin and rhodopsin photoreceptor excitation based on the spectrum of FIG. 1B. Key: Solid line: Balanced spectrum (Reference). Black dashed line: Melanopsin increase. Grey dashed line: Melanopsin decrease. Black dotted line: Rhodopsin increase. Grey dotted line: Rhodopsin decrease. Black dash-dot line: Melanopsin and rhodopsin increase. Grey dash-dot line: Melanopsin and rhodopsin decrease.

FIG. 3: is a schematic diagram showing one embodiment of a device according to the invention including control and emission elements.

FIG. 4: is schematic diagram showing one embodiment of a system according to the invention using exemplars for a visual display and video projector.

FIGS. 5A and 5B: schematic diagrams showing one embodiment of a computer and computer processor according to the invention.

FIG. 6: illustrates a three-dimensional CIE 1931 x,y,Y chromaticity diagram, showing that gamut reduces as relative luminance increases. It also includes data from refereed scientific studies that shows that rhodopsin (square symbols) and melanopsin (circle symbols) contribute to both color and luminance perception.

FIGS. 7A and 7B: show that within the CIE 1931 chromaticity diagram, the Planckian locus appears as an arc representing a standardised series of ideal and measured spectra (blackbody radiators; shown from 2,000K to 10,000K) intended to reference ‘white’ light (e.g. sunlight) in various conditions and times of day. FIG. 7A shows the region highlighted with arrows in FIG. 7. The symbols correspond to four different white light spectra—sunlight (circle symbol), generic white LED (square symbol), red, green and blue LED (cross symbol) and an embodiment of the biologically directed light (diamond symbol)—described in detail in the following.

FIG. 8: shows three different light spectra, scaled for equal-luminance. Dotted line for sunlight; dashed line for a generic white LED; solid line for a red, green and blue LED. These spectra share the same correlated color temperature (CCT=5,000K) and the same chromaticity coordinates (x=0.346, y=0.359) on the Planckian locus of the 1931 CIE color space. Referring back to FIGS. 7A and 7B, the circle symbol is for sunlight, square symbol for the white LED, and cross symbol for the RGB LED.

FIG. 9: illustrates a physiologically-defined color space representing the melanopsin and rhodopsin stimulation of a light (solid line shows the photoreceptor locus, dashed line shows the blackbody radiator between 2,000K and 10,000K). This melanopsin and rhodopsin space clearly shows that two different LED spectra (white LED and RGB LEDs from FIG. 8) that produce the same cone stimulations (FIG. 7, square and cross symbols), also produce two different physiological melanopsin and rhodopsin responses (FIG. 9, square and cross symbols), with deviations away from the Planckian locus.

FIG. 10: shows yet another component light spectra emitted from another embodiment of a biologically directed lighting device according to the invention, the device having a spectral output (5,000K) that actively considers all five photoreceptor classes (FIG. 10, dotted line), the effect of this light on the stimulation of the 3-cone opsins (FIGS. 7A and 7B, diamond symbol), rhodopsin and melanopsin (FIG. 9, largest diamond symbol) matches that of the sun (FIG. 9, circle symbol; FIGS. 7 and 7A, circle symbol). With a spectral output that considers only four photoreceptor classes (e.g. 3-cone opsins and melanopsin: Figure dashed line), there is a systematic deviation away from the Planckian locus and a different physiological effect to that from 5,000K sunlight stimulation (FIG. 9, middle sized diamond vs. circle). With a spectral output that considers only the three cone photoreceptor classes (FIG. 10, solid line), there is a systematic deviation away from the Planckian locus and a different physiological effect to that from sunlight stimulation on the melanopsin and rhodopsin photoreceptors (FIG. 9, smallest diamond symbol vs. circle).

FIGS. 11A; 11B; and 11C: FIG. 11A shows a spectrum generated by a device according to one embodiment of the invention that is metameric across all five photoreceptors to that of a 5,000K blackbody. FIG. 11B shows a spectrum generated by a device according to one embodiment of the invention that that is metameric across all three cone types to that of a 5,000K blackbody, and metameric for rhodopsin and melanopsin to that of a 6,000K blackbody. FIG. 11C shows a spectrum generated by a device according to one embodiment of the invention which applies a color correction for the color variation induced by the difference in rhodopsin and melanopsin excitation, relative to reference cone excitation.

FIGS. 12A; 12B; 12C; and 12D: show spectra generated by a device according to one embodiment of the invention that show a match of the photoreceptor-to-photopic luminance excitation ratio for the five photoreceptors that align with the blackbody radiator at the specified CCT (degrees Kelvin) (black solid line; CRI>97), that show a match of the photoreceptor-to-photopic luminance excitation ratio for all three cone types to that of a blackbody radiator at the specified CCT and for rhodopsin and melanopsin to that of a lower CCT (grey solid line; CRI>85), and that show a match of the photoreceptor-to-photopic luminance excitation ratio for all three cone types to that of a blackbody radiator at the specified CCT and for rhodopsin and melanopsin to that of a higher CCT (black dash line; CRI>80). Example CCTs specified as 3500K (FIG. 12A), 4000K (FIG. 12B), 5000K (FIG. 12C) and 6500K (FIG. 12D).

FIG. 13: a plot showing the photoreceptor-to-photopic luminance excitation ratio for the five photoreceptors and the bounds of their defined ranges (shading) as a function of CCT (degrees Kelvin) according to one embodiment of the invention.

FIGS. 14A and 14B: graphs showing a comparison between the emitted light produced according to one embodiment of the invention and a commercially available product, the Philips Hue product. FIG. 13A shows cone photoreceptors, while FIG. 13B shows rhodopsin (circle symbols) and melanopsin (square symbols).

FIGS. 15A and 15B: graphs showing a comparison between the emitted light produced according to one embodiment of the invention and a commercially available product, the SORAA product. FIG. 14A shows cone photoreceptors, while FIG. 1 shows rhodopsin (circle symbols) and melanopsin (square symbols).

FIG. 16A shows a device according to one embodiment of the invention that generates spectra with defined photoreceptor excitation ratios for supplemental light therapy. FIG. 16B shows a device according to another embodiment of the invention that generates biologically balanced light spectra to match the photoreceptor-to-photopic luminance excitation ratio for the five photoreceptors that align with the blackbody radiator at the specified CCT, or with variable photoreceptor-to-photopic luminance excitation ratios for the five photoreceptors with the perceived colour appearance matching the daylight CCTs, or with variable photoreceptor-to-photopic luminance excitation ratios for the five photoreceptors prompting a biological response matching that of a different blackbody radiator with changes that are invisible to the eye (i.e. chromaticity invariant) or difficult to perceive.

Skilled addressees will appreciate that elements in the drawings are illustrated for simplicity and clarity and have not necessarily been drawn to scale. For example, the relative dimensions of some elements in the drawings may be distorted to help improve understanding of embodiments of the present invention.

### DETAILED DESCRIPTION OF THE INVENTION

The present invention relates to a device, method and system for biologically balanced artificial light.

The invention is at least partly predicted on the inventors' unexpected discovery that biologically balanced artificial light which provides light that activates all ocular photoreceptors may positively benefit behaviour and/or may not have the disadvantages of existing illumination methods and devices which disrupt circadian rhythm or negatively affect mood or sleep-wake patterns. The invention may have additional advantages including one or more of promotion of wakefulness, increased autonomic nervous system arousal and improved sleep.

At least part of the invention lies in the inventors' recognition of the problem that biologically designed lighting in homes and offices is not a mature technology. Artificial lights, including those in computer devices, for example screens and phones, can cause circadian disruption and negatively affect mood and/or sleep-wake patterns. This effect of light on behaviour is as significant as diet and exercise. In one embodiment, the inventors have created an over-determined multi-primary lighting system using a method that may advantageously afford one or more of: (α) a precise manipulation of a person's visual perception and biological rhythms that is linked to industry defined standard human observer sensitivity functions; (β) a broader band spectral distribution to provide a truer representation of the natural environment; and (γ) a solution that provides a biologically balanced light with changes that are invisible to the eye (i.e. chromaticity invariant) and directed to the circadian system.

The prior art does not principally approach device design and functionality from a physiological perspective (e.g. color appearance as a by-product of design, rather than the goal) thereby limiting capability to the generation of artificial light spectra within their defined metrics (e.g. 1931 CIE chromaticity diagram and/or Melanopsin Effectiveness Factors). By not accounting in the metrics and device design for the stimulation of rod photoreceptors by the light spectrum, the perceptual hue, saturation and/or brightness contributions from melanopsin-containing ipRGCs and/or rods in response to a light spectrum, and the contribution of rods to circadian entrainment, the prior art leaves gaps where standard metrics may remain the same, but the output spectrum of the device, or its biological effects, may change. As such, should any of the defined metrics in the prior art be incorrect in the representation of the physiological effects of the light, the prior art may not inherently have the capacity to adapt. Moreover, output spectrums defined by different metrics will lead to different biological responses. This invention describes a device that, in one embodiment, may be capable of producing an output spectrum that matches those of prior art, but is not limited to those spectra in maintaining its own described specifications and capabilities. The device controls stimulation of all ocular photoreceptors in defined ratios. In comparison, the prior art can produce only a subset of this lighting device, one that lacks full photoreceptor control, for example 5,000K with a defined melanopsin excitation, but not rhodopsin, and not with color correction.

Biologically balanced or human centric lighting can be contrasted with conventional artificial light sources which have been developed irrespective of whether or not it was the “right” biological light. In one embodiment, the method, device and system provides biologically balanced or human centric light by producing illumination that actively stimulates all ocular photoreceptor classes comprising the melanopsin-containing ipRGCs; rods; and the 3 cone types L-, M-, and S-. Significantly, the stimulation of each respective ocular photoreceptor class may be in a defined ratio.

Standard controllable RGB lighting generates white light that tends to result in an overstimulation of melanopsin and rhodopsin and is therefore not capable of providing natural modulation of melanopsin, rods, and LMS-cones during the approximately 24-hour daylight cycle. The inventors' biologically designed lighting may be used to produce balanced, photoreceptor-directed changes in the melanopsin, rod and cone photoreceptor ratios during the circadian cycle to promote wakefulness, increased autonomic nervous system arousal, and sleep.

Another way of characterising the invention, is that the inventors are the first to recognise that the prior art systems are limited because they only vary the melanopsin activity relative to the activity of photopic luminance which may activate only L- and M-cone opsins. The prior-art systems are not capable of modulating rods and/or melanopsin and rods independent of the three cone photoreceptor classes.

The term “CIE” as used herein refers to the International Commission on Illumination, which is abbreviated CIE for its French name, Commission Internationale de l'éclairage.

Light is electromagnetic radiation capable of producing visual and/or non-visual responses. The SI (International System of Units, Système international (d'unites)) unit for luminance is candela per square metre (cd·m⁻²). The term “luminance” is the photometric analogue of radiance. Photopic Luminance is calculated by integrating the spectral energy distribution (radiance) of a source with a standard luminous efficiency function V(λ) multiplied by a conversion factor that relates lumens to watts.

As used herein, the terms “luminosity function” and “luminous efficiency function” are used to describe the standard photopic luminous efficiency function V(λ) and the CIE scotopic luminosity function (V′λ). The scotopic function (V′λ) represents the rhodopsin spectral sensitivity. Luminance is sometimes loosely related to perceptual brightness (the term “brightness” may be used in this context).

As used herein the term “spectral component” is used to refer to a component part or portion of the electromagnetic radiation emitted such as, a component part or portion of the total electromagnetic spectrum emitted by a light source.

As used herein the term “blackbody” is an idealized physical body that absorbs all incident electromagnetic radiation and emits radiation with a spectrum dependent on its temperature. A blackbody spectrum for a given CCT may be calculated as per CIE or TM-30 definitions.

The term “gamut” is used herein to refer to a certain complete subset of photoreceptor activation ratios. A common usage of gamut refers to the subset of chromaticities which can be represented in a given circumstance, such as within a given color space or by a certain output device.

As used herein the term “metameric” is used to describe two lights with different proportions of energy at certain wavelengths which produce the same excitation of the three cone types. This may be extended to include melanopsin and/or rhodopsin for the purpose of this document.

As used herein the terms “smlri” and ““SMRLI” and the individual component letter references are used to refer to the ocular photoreceptors, “s” or “S” referring to the short wavelength sensitive cones; “m” or “M” referring to the medium wavelength sensitive cones; “l” or “L” referring to long wavelength sensitive cones, “r” or “R” referring to rhodopsin; and “i” or “I” or “ipRGC” referring to melanopsin containing intrinsically photosensitive retinal ganglion cells (ipRGCs).

As used herein “RGB” is an acronym for red, green and blue, a common LED package.

As used herein the term “overdetermined” means to determine, account for, or cause (something) in more than one way or with more conditions than are necessary. In the context of mathematics, a system of equations is considered overdetermined if there are more equations than unknowns, which often results in multiple solutions.

The Standard Observer is a reference for specifying the sensitivity of the human eye to light. As used herein “Standard Colorimetric Observer” or “Standard Observer” is used to mean one or more sensitivity functions to standardize the representation of an ideal observer whose color matching properties represent a CIE color matching function. The Standard Observer function may be a 2 degree CIE Standard Colorimetric Observer (small field) or a 10 degree CIE Supplementary Standard Colorimetric Observer (large field), or these functions modified to represent variations in sensitivities. Although not currently standardised, this could be extended to equivalent functions in animals.

Light is electromagnetic radiation capable of producing visual and/or non-visual responses. As used herein the terms “light” and “electromagnetic radiation” may be used interchangeably. The term “electromagnetic radiation” is broader than the term “light” and refers to the waves, or their quanta or photons, of the electromagnetic field, propagating or radiating through space, carrying electromagnetic radiant energy. Electromagnetic radiation includes radio waves, microwaves, infrared, visible light, ultraviolet, X-rays, and gamma rays. Light is used to mean electromagnetic radiation within a portion of the electromagnetic spectrum and includes visible light, which is the visible spectrum that is visible to the human eye and is responsible for the sense of sight.

The term “correlated color temperature” (CCT) is a parameter that can describe a given chromaticity, as referenced to the chromaticity of the nearest blackbody radiator.

The term “photopic vision” is used herein to mean the vision of the eye under well-lit conditions (luminance levels above several cd·m⁻²).

As used herein the term “photoreceptor-to-photopic luminance activation ratio” means the ratio of the excitation of a photoreceptor class (e.g. S, M, L, R or i) caused by a given light spectrum, to the excitation of the photopic luminous efficiency function (V(k)) caused by the same spectrum. This term may be used interchangeably with ‘Photoreceptor excitation’ or ‘Photoreceptor ratio.’

These values are defined as being unit-normalised to equal-luminance, using one or more sensitivity functions. The coefficients and bound limits are defined relative to the earlier stated sensitivities but may be varied if different functions are used. The polynomials generally describe a series of blackbody radiators correlated with a set of sensitivity functions and optionally normalised.

Within the context of this document, the term “contrast” refers to the difference between photoreceptor class excitations caused by two light spectrums. Typically this is given as Weber contrast, defined by the equation (P−Q)/Q where P is a photoreceptor class excitation for a given spectrum and Q is the same class excitation for another spectrum.

As used herein the term “biologically directed lighting” or “biologically balanced lighting” is used to mean illumination that actively stimulates all ocular photoreceptor classes comprising the three cone types L-, M-, and S-; rhodopsin; and melanopsin-containing ipRGCs in a desired ratio.

To exert the full effect of light on human or other animal circadian rhythms and sleep, an artificial light source must control the relative activity of all five photoreceptor classes in the human eye (melanopsin-driven intrinsically photosensitive Retinal Ganglion Cell (ipRGC) responses; rhodopsin-driven rod responses; and the three opsin-driven cone responses) with their different spectral sensitivities to light (References: Feigl & Zele 2014; and Adhikari, Zele & Feigl 2015). While current lighting systems focus on the activation of melanopsin expressing ipRGCs alone (through “melanopic” light), ipRGCs do not transmit the full effect of light to the higher brain centres for photoentrainment. Based on scientific evidence (Reference: Altimus et al 2010; and Dumpala, Zele & Feigl 2019), ipRGCs access retinal circuitries through rod and cone pathways to relay light information at all light levels for circadian entrainment and for the full effect of light on sleep and wakefulness. By having all five photoreceptor classes work together, the present invention may extend the range of light intensities necessary for photoentrainment—adjusting the suprachiasmatic nucleus to a 24-hour day/night cycle.

In one embodiment, the present inventors have provided an all-purpose artificial lighting system that is biologically-directed for synchronizing circadian rhythms, sleep, wake and arousal with the 24 hour day-night cycle. The device, method and system of the invention may be used in homes and in commercial settings.

Advantageously, the invention may provide any required modulation synchronization, for example if the work environment requires increased alertness for night shift workers. This may lead to increased workplace productivity and safety. All changes may occur without visible perceptible changes in stimulus color, i.e. chromaticity, or any change may produce the biological-directed modulations while incorporating a change in the spectral content of the light to mimic the natural variation in the solar day such as, cooler (i.e. bluer) during the day and warmer (i.e. more orangish) during the evening.

Limitations of the methods described in the prior art include that light spectrums developed to only modulate melanopsin activity ultimately fail to distinguish that the fundamental light-dependent physiological processes required for normal humans photoentrainment (sleep and wakefulness circadian behaviours) are supported by the activity of both melanopsin- and rhodopsin-mediated photoreception, in addition to the cone-mediated photoreception (Reference: Altimus et al 2010). In other words, the full effects of light on human circadian rhythms is dependent on the activity of all five photoreceptor classes in the human eye.

Another limitation of the prior art is the use of metrics to quantify the activity of the light with reference only to melanopsin (MEF) and photopic luminance (M/P ratio). These metrics correlate a given spectrum with melanopsin and photopic luminance responses respectively, and then find the relative activation levels. These metrics oversimplify the visual process by ignoring the role of the S-cone visual pathways on color appearance, ignoring the perceptual differences within the photopic luminance function itself, and ignoring the effects of rod photoreception on both vision and circadian rhythms. This results in those lighting systems suffering from inadvertent variations in color appearance (or color fidelity) of the “white” light (e.g. CRI, CIE-Ra). Moreover, the MEF does not account for changes in appearance of a white light (both brightness and color) due to variation in the level of melanopsin activity (Reference: Zele, Feigl et al 2018; and Cao et al 2018). Neither the MEF nor M/P ratio consider this when light is specified with reference to the CCT. This is in addition to the color variation inherent in the CCT metric. In one embodiment, the present invention uses five or more dimensions to define the resultant spectrum, where three are currently directly linked to current trichromatic color theory (LMS) and there is scientific evidence that the remaining two photoreceptors (melanopsin expressing ipRGCs and rods) contribute to color perception (Reference: Cao, Zele & Pokorny 2008; Cao, Pokorny, Smith & Zele 2008; Zele, Feigl et al 2018; and Zele, Adhikari et al 2019). This is one purpose of the color correction functionality of the invention.

Yet another limitation of the prior art is that no prior art device includes a design for an artificial lighting fixture and/or system that, by way of fine spectral manipulation, simultaneously controls the activity of the melanopsin-containing ipRGC, rods and three cones to exert a more comprehensive influence over circadian rhythms while achieving a visually appealing natural light spectrum. That is, in one embodiment, the invention corrects for the change in color appearance of the white spectrum arising due to the requisite change in the relative activity of the five photoreceptors that support the circadian rhythm during the solar day/night. The current invention uses a physiologically based quantification of the light to control the activity of all five known photoreceptor classes in the human eye.

In one embodiment the invention provides a physiologically-designed “white” light that is referenced to the activity of all five photoreceptor classes and provides precise control of the melanopsin-containing ipRGCs and rods for exerting the full-effects of sleep and wakefulness/alertness on our circadian rhythms.

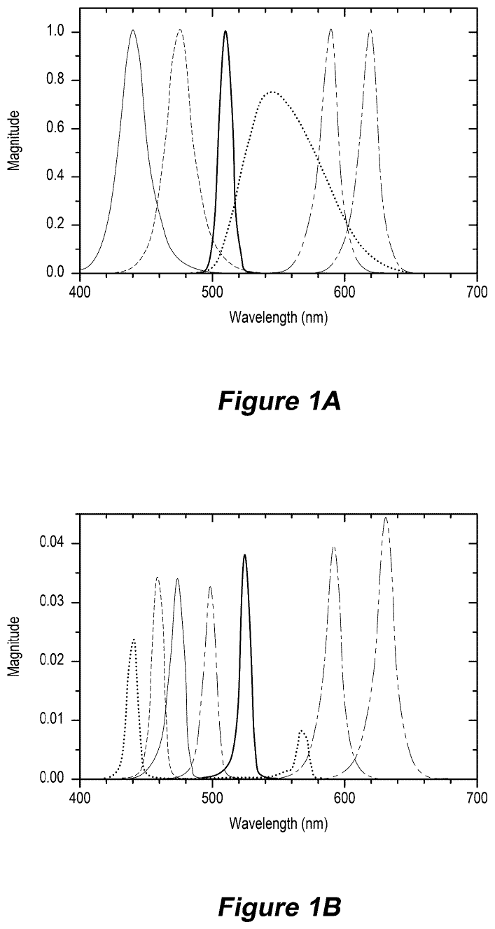

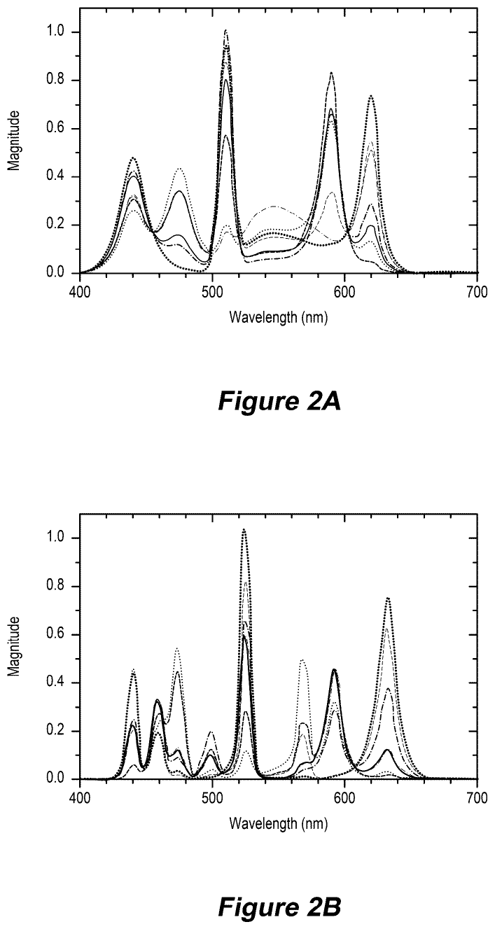

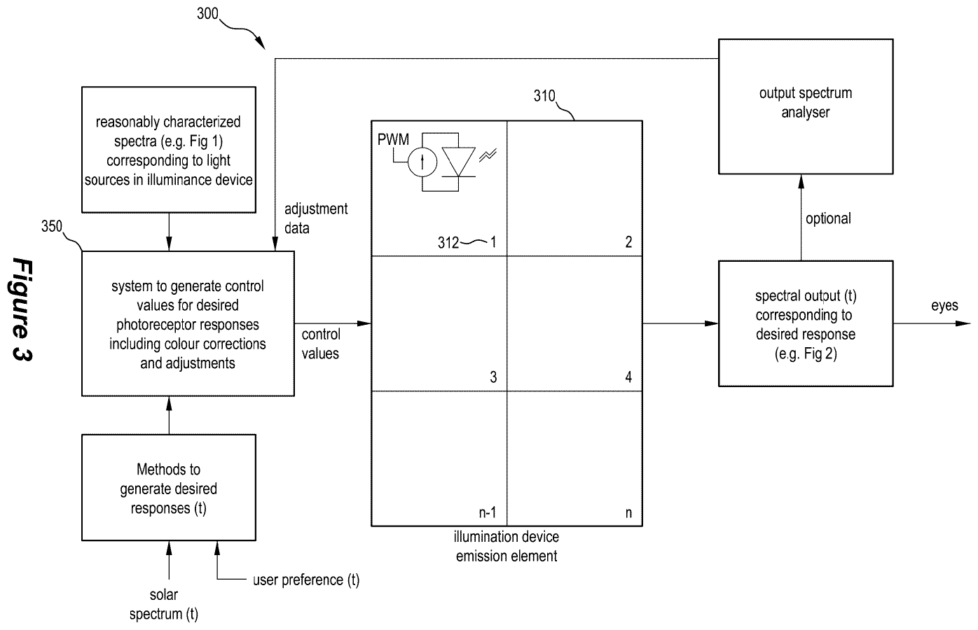

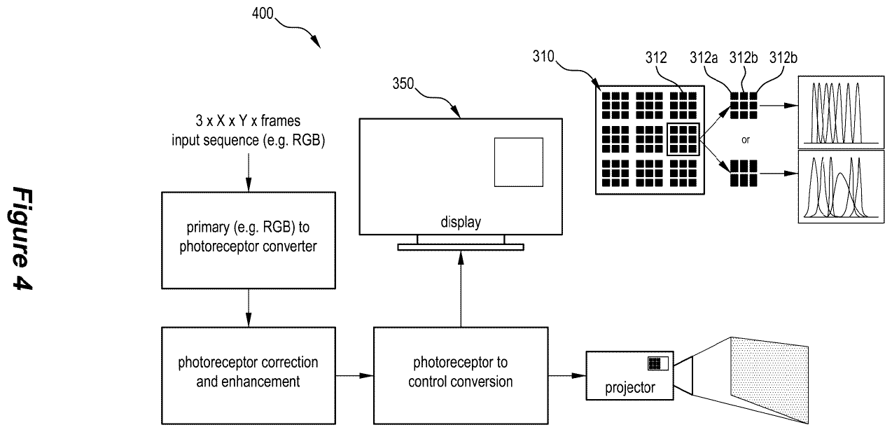

In another embodiment, the invention provides a minimisation algorithm (optimization) to compute spectral outputs based on individual photoreceptor responses. The invention may optimize the spectral output of a reasonably-characterised set of light sources such as, shown in FIGS. 1A and 1B to achieve a combined spectrum based on individual photoreceptor responses, see FIGS. 2A and 2B, and color correction preferences.

In the example shown in FIG. 2A, the balanced spectrum (solid line) is set to provide a spectrum metameric to an equal energy white. All other 6 spectrums share the same L-, M- and S-cone responses and therefore the same CCT (˜5300K). Trichromatic color theory describes light in terms of the activation of these three (L, M- and S-cone) photoreceptors and does not account for any contribution to color appearance resulting from the differential activation of melanopsin-containing ipRGCs and/or rods. With reference to the balanced white spectrum (solid line, 2:1 L:M cone ratio with S=1) the theory predicts that if these ratios remain the same, independent of change in the activation of the melanopsin-containing ipRGCs and/or rods (other lines) then color appearance of the white light will also remain the same. This is not the case however, as evidenced by data demonstrating that an increase in rod excitation (Reference: Cao, Pokorny, Smith & Zele 2008) or a decrease in rod excitation relative to a balanced spectrum, can both cause a change in color appearance (Reference: Cao, Zele & Pokorny 2008); in addition, the change in melanopsin excitation causes a change in color appearance (Reference: Zele, Feigl et al 2018; Zele, Adhikari, Cao & Feigl 2019; and Cao, Chang & Gai 2018) and an increase in perceived brightness (Reference: Zele, Adhikari, Feigl & Cao 2018). Therefore the trichromatic theory does not provide a satisfactory framework to evaluate the appearance of the lights and so, in one embodiment, the invention provides a correction method (incorporated in FIG. 3) to alter how the light spectrum changes the three (L, M- and S-) cone photoreceptors activations, to nullify the perceptual change in appearance of the light, while maintaining the appearance of the balanced spectrum during the change in melanopsin-containing ipRGC and/or rod activity.

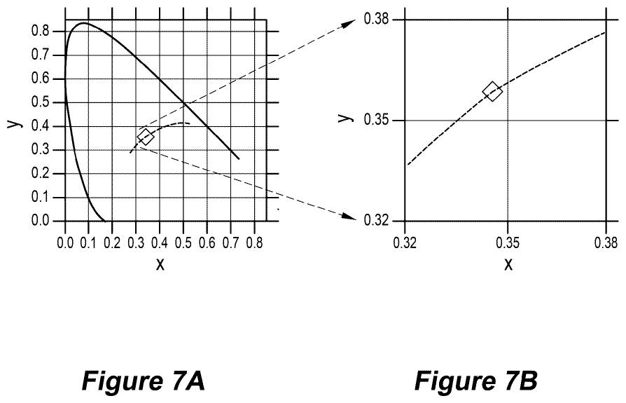

The two-dimensional CIE 1931 diagram (FIGS. 7A and 7B) representing chromaticity's of equal-luminance can be linearly transformed to other known color spaces (e.g. perceptually uniform CIE 1976 L* u* v*; chromaticity diagrams with physiological axes representing L- M- and S-cone excitation) providing it is referenced to a blackbody radiator (spectrum) for the same time of day. The linear transformation to a L- M- and S-cone space means that the CIE 1931 representation is the equivalent of a cone-photoreceptor-only representation of color. As circadian entrainment is driven by at least one non-cone photoreceptor class, this color space (and by extension, CCT) is an inadequate representation of the effect of biologically-driven light on vision and circadian rhythms.

The invention also makes possible real-time correction for changes in the spectral quality of the light. The changes may comprise one or more deviation from visually acceptable “white” light relative to that achievable by the prior-art by adjusting the activity of the three cone-opsins resulting from the change in CCT. As shown with reference to FIG. 3, this may be achieved by choosing light spectra that physiologically vary the activity of the cones for the purpose of correcting for color and brightness variation induced by modulation of the melanopsin-containing ipRGCs and rods in order to generate a biologically effective artificial light to support circadian rhythms, including sleep and wakefulness, in a manner similar to the natural environment.

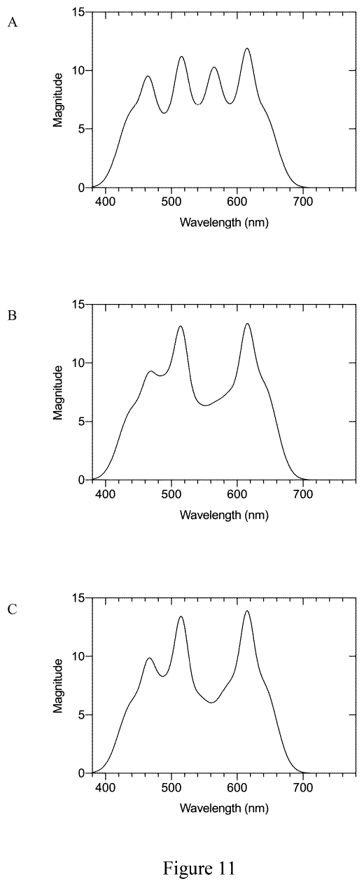

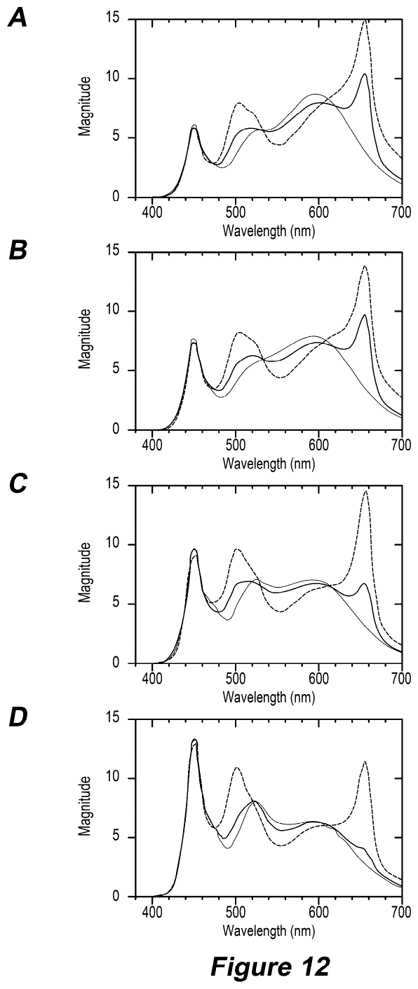

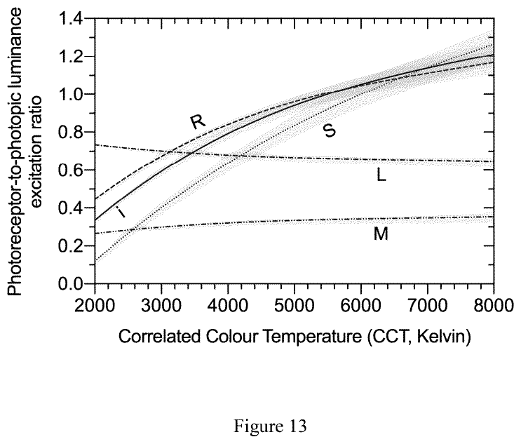

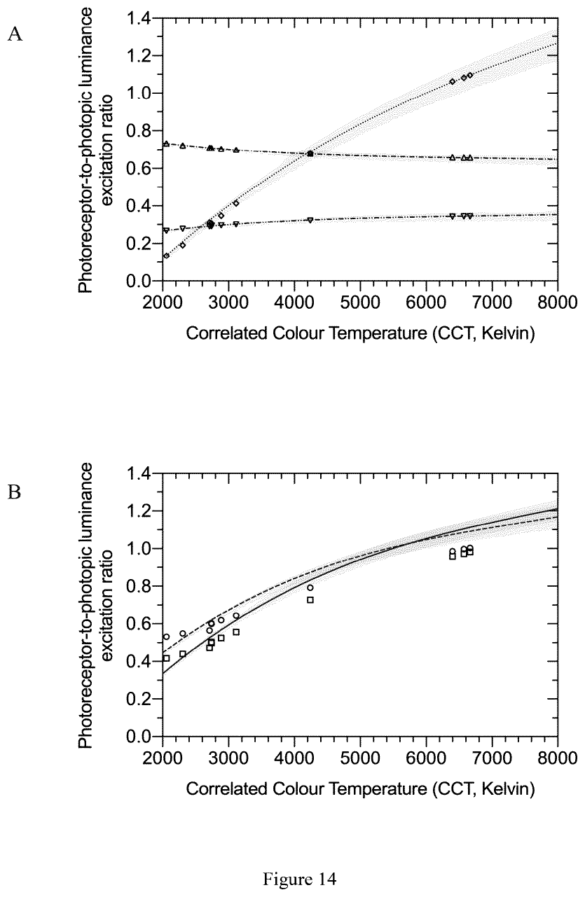

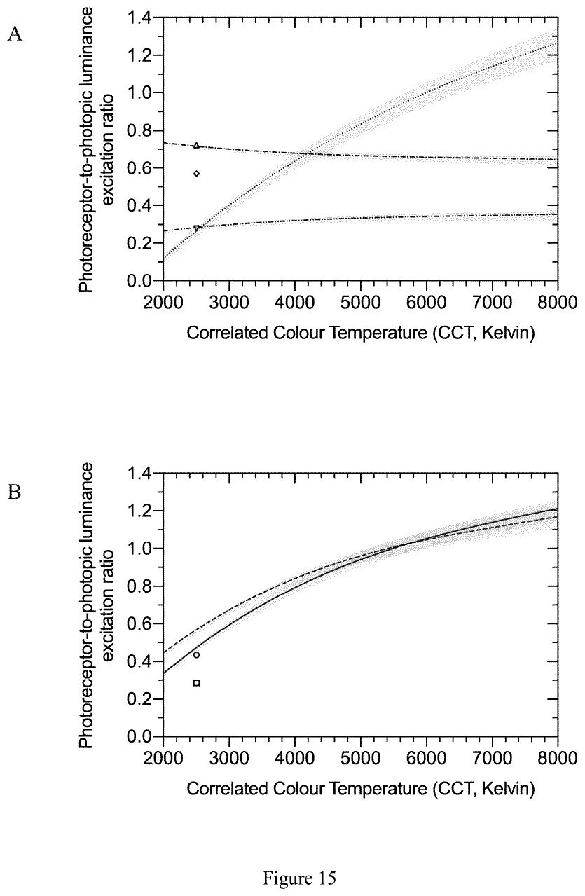

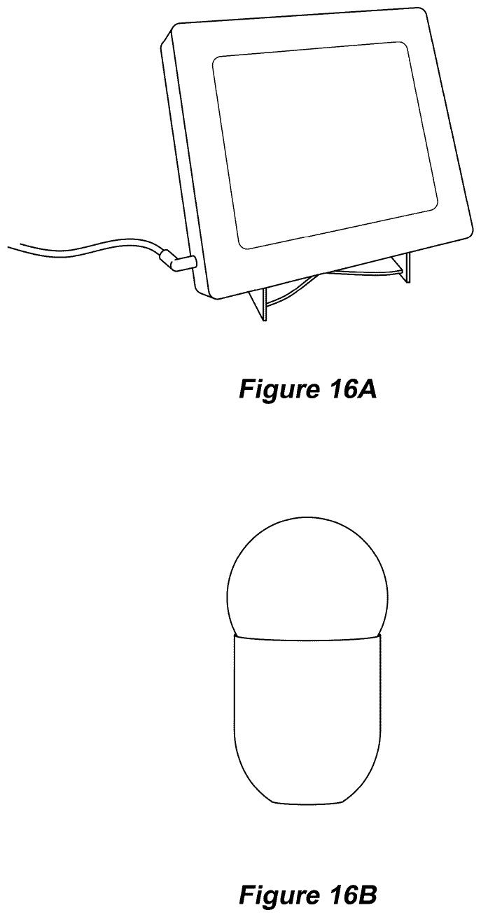

Another advantage of the present invention is that the CCT may remain constant during a 24-hour cycle, which may be suitable for some solar-cycle independent applications. By providing a dynamic or real-time control of melanopsin-containing ipRGCs and rods any realisable contrast level (flux) relative to constant (or variable) cone activity may be provided. The color invariance is achieved by correction of the color changes caused by melanopsin-containing ipRGC and/or rhodopsin activation (FIG. 11).

Yet another advantage of the invention is that color temperature may be varied such as, from low to high CCT, during the solar day while providing the required change in activity of the melanopsin-containing ipRGCs and rhodopsin to modulate the circadian rhythm, with the color correction negating the perceptual changes arising from these photoreceptor activations.

The independent modulation of the photoreceptor activity means the invention is not limited to decreasing melanopsin activity by dimming the light, which is a limitation of the prior art. The transition between photoreceptor states may be invisible to the eye and may not require any change in ambient illumination. This is important in a setting requiring constant ambient illumination, but variable states of circadian activity.

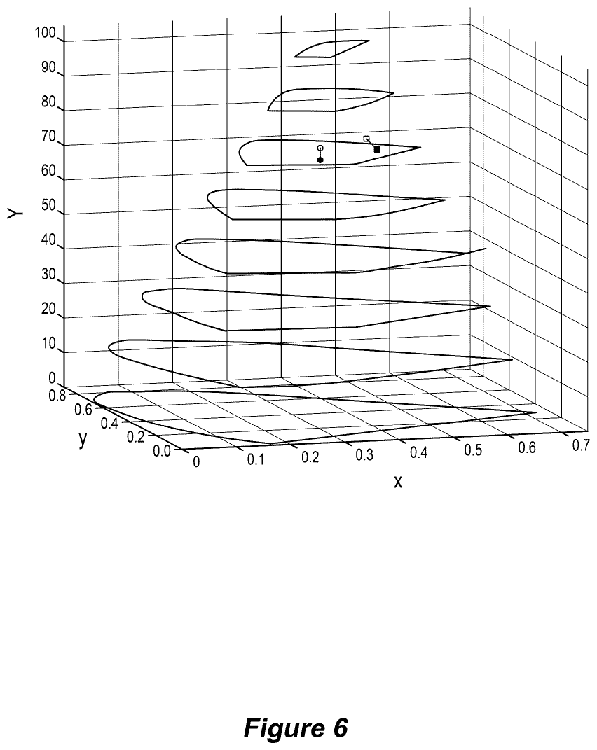

The gamut of a three-dimensional CIE 1931 x,y,Y chromaticity (FIG. 6) diagram reduces as relative luminance (Y) increases (shown from Y=0 to Y=100). Two ‘white’ lights with different cone chromaticities (FIG. 6, closed circle and closed square) are represented at a nominal relative luminance of 75 (FIG. 6, third x,y plane from the top). Increasing the melanopsin excitation of a light spectrum (open circle) causes a change in the perceived chromaticity (i.e. change in the color appearance away from the nominal ‘white’) and luminance (i.e. increase in brightness) of the light (Reference: Zele, Feigl et al 2018). Increasing the rhodopsin excitation of the light spectrum (open square) also causes a change in perceived chromaticity (i.e. change in the color appearance away from the nominal ‘white’) and luminance (i.e. increase in brightness) (Reference: Cao, Pokorny, Smith, Zele 2008). This invention describes a device that is capable of changing the ratio of the cone photoreceptor stimulations independently of melanopsin-containing ipRGCs and rods such that the perceived difference resulting from melanopsin-containing ipRGCs and/or rod modulation is minimised. Moreover, the correction for the increase in relative luminance (Y) of the light due to the higher melanopsin and/or rhodopsin excitation (the line connecting the closed and open symbols in FIG. 6) can be correlated with an overall decrease in energy output (dependent on the component spectra used). The magnitude of this vector is dependent on the color temperature and the melanopsin and/or rhodopsin excitation (relative to the reference) of the light spectrum. The algorithm that may be part of the device is capable of choosing a spectral output that minimizes the energy required for a given perceived brightness.

Within the CIE 1931 chromaticity diagram (FIGS. 7A and 7B), the Planckian locus appears as an arc representing a standardised series of ideal and measured spectra (blackbody radiators; dashed line shown from 2,000K to 10,000K) intended to reference ‘white’ light (e.g. sunlight) in various conditions and times of day. The Correlated Color Temperature (CCT) in degrees Kelvin (K) of a light source can be determined by its proximity to a point on the Planckian locus. A lower CCT (e.g. 2500K) represents a ‘white’ appearing light with an orangish-reddish hue; a higher CCT (e.g. 6,000K represents a ‘white’ appearing light with a bluish hue. This means that perceptually-invariant light (e.g. a nominally, preferably ‘white’ appearing light) cannot be defined solely by its CCT. There is shown a circle symbol for sunlight, square symbol for the white LED, cross symbol for the RGB LED).

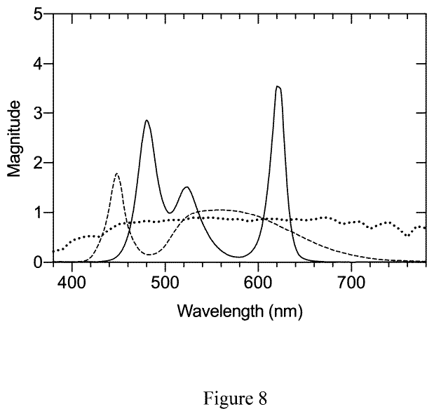

Three different light spectra, scaled for equal-luminance (FIG. 8: dotted line for sunlight; dashed line for a generic white LED; solid line for a red, green and blue LED), share the same correlated color temperature (CCT: 5,000K) and the same chromaticity coordinates (x=0.346, y=0.359) on the Planckian locus of the 1931 CIE color space (see FIGS. 7A and 7B where circle symbol is for sunlight, square symbol is for the white LED and cross symbol is for the RGB LED). These three different example light spectra are also known as metamers because they have the same 1931 CIE X, Y chromaticity coordinates and luminance (or same L-, M- and S-cone excitations).

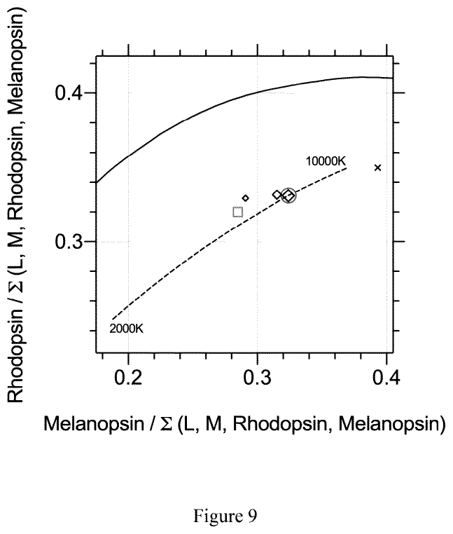

Creating metamers tends to focus only on the cone space, which means a color can be considered the same while leaving two photoreceptor classes as variables. For the purpose of recreating a truly comparable biological and perceptual response, a metamer must keep the same excitation across all five photoreceptor classes. Using the same blackbody radiators as for the 1931 CIE space, we include a physiologically-defined color space representing the melanopsin and rhodopsin stimulation of a light (FIG. 9, solid line shows the photoreceptor locus, dashed line shows the blackbody radiator between 2,000K and 10,000K). This melanopsin and rhodopsin space (FIG. 9) clearly shows that two different LED spectra (white LED and RGB LEDs from FIG. 8) that produce the same cone stimulations (FIG. 7, square and cross symbols), also produce two different physiological melanopsin and rhodopsin responses (FIG. 9, square and cross symbols), with deviations away from the Planckian locus. In terms of the 1931 CIE space, variations in melanopsin and rod excitations are generally evident as changes in the CRI and/or gamut. FIG. 9 demonstrates that there are complex relationships within the spectra that cause differential changes in the photoreceptor responses and their biological effects, and that ignoring any of the five photoreceptor classes when creating color will generally yield a suboptimal result.

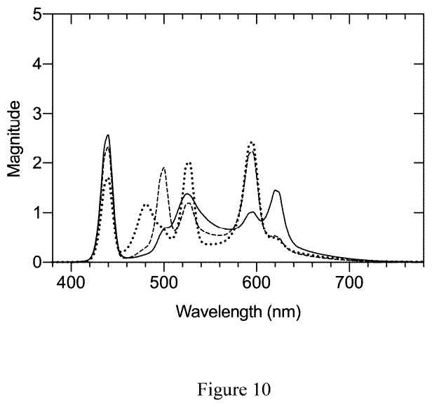

As an example, for a biologically directed lighting device with a spectral output (5,000K) that actively considers all five photoreceptor classes (FIG. 10, dotted line), the effect of this light on the stimulation of the 3-cone opsins (FIG. 7, diamond symbol), melanopsin and rhodopsin (FIG. 9, largest diamond symbol) matches that of the sun (FIG. 9, circle symbol; FIG. 7, circle symbol). With a spectral output that considers only 4 photoreceptor classes (e.g. 3-cone opsins and melanopsin: FIG. 10, dashed line), there is a systematic deviation away from the Planckian locus and a different physiological effect to that from 5,000K sunlight stimulation (FIG. 9, middle sized diamond vs. circle). With a spectral output that considers only the 3 cone photoreceptor classes (FIG. 10, solid line), there is a systematic deviation away from the Planckian locus and a different physiological effect to that from 5,000K sunlight stimulation on the melanopsin and rhodopsin photoreceptors (FIG. 9, smallest sized diamond symbol vs. circle). Additionally, the color contribution of melanopsin and rods has not been standardised, and so may require the capability to effect excitation of all five photoreceptor classes independently to achieve perceptually invariant light. For this reason, one embodiment of the biologically directed light device is capable of generating spectral output that actively considers all five photoreceptor classes which allows for active color correction (or the contributions from melanopsin and rods) and color rendering (high CRI) if the user intends to deviate from ‘natural’ photoreceptor ratios (see the photoreceptor and spectra ratios discussed below). If a user requires a preferable ‘white’ that is not positioned on the Planckian locus, another color correction can be applied for the desired rhodopsin and/or melanopsin stimulation chosen by the user.

In one embodiment the device can generate spectra with correlated color temperatures (CCT) ranging between 3,000K and 7,000K (referenced to a normalised black body radiator; see Table 3, which provides CCT values in 1,000K steps).

FIGS. 3 and 4 show embodiments of a device 300 and system 400 according to the invention comprising an illumination source 310 comprising one or more light emitter 312 and a light emitter controller 350. The one or more emitter 312 emits electromagnetic radiation that activates all ocular photoreceptors in a desired ratio. The one or more emitter 312 contains a plurality of emitters 312, or a singular emitter 312 with variable spectral output. Each emitter 312 may contain one or more physical elements capable of emitting electromagnetic radiation primarily within the visible light spectrum, but possibly outside. Each emitter 312 may have a reasonably characterised spectral output such as, to one of the spectra shown on FIG. 1A or 1B, for a given control value corresponding to that component's input.

The light emitter controller 350 may control the illumination source 310 to emit the electromagnetic radiation that actively stimulates all ocular photoreceptor classes.

Device 300 and system 400 provide active stimulation of all ocular photoreceptor classes directly due to the emitted electromagnetic radiation that is emitted from the one or more emitter 312. Advantageously, the active stimulation of all ocular photoreceptor classes may be in a defined ratio which for example, may be stimulation correlated to a part of a solar day. The stimulation correlated to a part of a solar day may be in synchronisation with the solar day at that location or time-shifted from the solar day at that location. This time-shift may be of assistance to shift workers or those recovering from or preparing for travel. The defined ratio may also be to a stimulation correlated to one or more dysfunctional photoreceptor class in the retina. This dysfunction could for example result from an ophthalmic disease or condition. The stimulation may comprise an increase in the one or more dysfunctional photoreceptor class activation to a normal or a functional level.

The provision of biologically balanced or human centric lighting also means the invention provides a method of photoentrainment; a method of treating an ophthalmic disease, disorder or condition; a method of treating a neurologic disease, disorder or condition; a method of treating metabolic disease, disorder or conditions; a method of treating a sleep disease, disorder or condition; a method of treating a mood disease, disorder or condition; a method of treating a circadian rhythm disease, disorder or condition; a method of promoting and/or aiding sleep or wakefulness; a method of supporting biorhythm; or a method of conserving energy during illumination comprising providing illumination specific to melanopsin and/or decreased (L+M) cone activation.

The one or more emitter 312 may emit electromagnetic radiation comprising a bandwidth between 420 and 650 nm or between 300 and 780 nm. The emitted electromagnetic radiation may comprise at least five distinctly controllable spectral components at 420 to 470 nm; 460 to 510 nm; 500 to 550 nm; 540 to 600 nm; and 580 to 650 nm. The emitted electromagnetic radiation may comprise a sixth distinctly controllable spectral component at 500 to 610 nm. These six emitted spectral components are shown in FIG. 1A.

Another particular embodiment is shown in FIG. 1B which shows eight spectral components. These eight spectral components, defined by peak wavelength and deviation from peak wavelength at half-maximum, are: 440±5 nm; 459±5 nm; 473±5 nm; 499±5 nm; 524±5 nm; 567±5 nm; 592±8 nm; and 632±8 nm. The relative spectral power is irrelevant for this example but may be relevant for achieving function for a specific application. The scaled component spectra may be homogenised before entering the target environment.

As the skilled person will appreciate from FIGS. 1A; 1B; 2A; and 2B, the term “spectral component” is used to mean a distinct range of electromagnetic radiation. Although one spectral component may overlap with one or more other spectral component, their distributions are distinguishable.

The one or more emitter 312 may comprise a plurality of emitters 312, such as emitters 312a; 312b; and 312c labelled in FIG. 4. Each of the plurality of emitters 312 emit light at one or more bandwidth between 420 and 650 nm or between 300 and 780 nm. The plurality of emitters 312 may comprise distinctly controllable emitters 312 emitting light at the above spectral components. The plurality of distinctly controllable emitters 312 may comprise a respective emitter 312 for each spectral component. The plurality of controllable emitters 312 may comprise a fewer number of emitters 312 than spectral components wherein at least one of the controllable emitters 312 emits electromagnetic radiation at two or more of the spectral components. The plurality of distinctly controllable emitters 312 may comprise a larger number of emitters 312 than spectral components wherein at least two of the controllable emitters 312 emits electromagnetic radiation to create one spectral component.

Five or more channels of electromagnetic radiation emission may be provided. The five or more channels may comprise respective channels to the above-listed distinct spectral components such as, the five channels of the five ocular photoreceptor classes; the six channels of FIGS. 1A and 2A; or the eight channels of FIGS. 1B; and 2B.

As mentioned above, emitters 312a, 312b and 312c are labelled in FIG. 4. FIG. 4 actually shows the illumination source 310 to comprise a plurality of emitters 312 comprising a set of arrays. Each array may be selected to provide a desired spectral component. The array in which emitters 312a, 312b and 312 are labelled is shown to comprise nine emitters 312 which can emit the electromagnetic radiation illustrated in the top graph of FIG. 4, showing nine respective spectral components. Another six emitter 312 array is shown which can emit the electromagnetic radiation illustrated in the bottom graph of FIG. 4, showing six respective spectral components.

Each array, bank or group, or a subset of the one or emitters 312 comprised in each array, bank or group, may be independently controlled.

For each CCT the light spectrum emitted by the device 300 may be metameric to the photoreceptor excitations produced by the corresponding black body radiator (Table 3, S,M,L,R,I).

Photoreceptor excitations can be specified in reference to the CIE Standard Observer functions, with L+M=1.

The device 300 is capable of producing spectra eliciting the same physiological response as the natural solar spectrum.

In one embodiment, the device output metameric to a blackbody radiator at a nominal CCT may achieve CRI's>=95 (see Table 3, CRI).

The component spectra ratios of one embodiment of the device 300 are specified with reference to a 12-bit scale (Table 3, S1-S9).

In one embodiment the device 300 may generate a fixed CCT light spectrum with photoreceptor excitations equivalent to a desired daylight CCT at a high CRI.

In another embodiment the device 300 may prompt biological effects (e.g. photoreceptor excitations influencing circadian rhythms) aligned to changes in the solar day by transitioning the device spectrum from low melanopsin and rhodopsin excitations in the morning (e.g. for a low blackbody CCT), to higher rhodopsin and melanopsin excitations during the day, to low values in the evening. The parameters prompting these biological effects can be aligned to seasonal variation and/or geographical location.

The biologically-directed light generated by the device 300 may generate a spectrum that prompts a set of rhodopsin and melanopsin excitations that may match an alternative time of day to those elicited by the current natural solar day in the geographical location and time of day of the user with minimal perceptual variation from the desired appearance. These spectrums can be set to a desired circadian entrainment pattern of the user, their individual sleep/wake and alertness preferences resulting from, for example, occupational requirements (e.g. shift-work) or travel (e.g. jet-lag).

Additionally, the device 300 may be capable of generating perceptually invariant changes in the rhodopsin and melanopsin excitations that may be higher and/or lower than a fixed blackbody radiator by combining rhodopsin and melanopsin excitations for the desired circadian effect with the cone excitations of the preferred blackbody radiator and subsequent corrections to account for color variation.

In one particular embodiment, the device 300 may contain 9 independently controllable component spectra. In another embodiment it may contain a different number of independently controllable component spectra. The component spectra may be normalised Gaussian with centres at 25 nm increments from 400 nm to 640 nm, with a combination of wideband (approx. 46 nm FWHM bandwidth) and narrowband distributions (approx. 23 nm FWHM). The device may be required to generate a spectrum that is metameric (across all five photoreceptors) to that of a 5,000K blackbody. The device may produce an output spectrum (FIG. 11A) for that purpose which achieves a CRI of >95. The component photoreceptor and spectra ratios (relative to photopic luminance) required to achieve a (sun) biologically directed light stimulation includes: S-cone=0.8148; M-cone=L-cone=0.6663; Rhodopsin=0.9608; Melanopsin=0.9409 with LED1=6.2245; LED2=4.2179; LED3=5.3482; LED4=5.9605; LED5=6.0993; LED6=4.5857; LED7=6.1699; LED8=6.2801; LED9=6.2355.

Color corrections may be applicable in embodiments such as those with 9 independently controllable component spectra. The precision of the color correction is highest with >=5 or more independently controllable component spectra.

The device 300 may be required to generate a spectrum that is metameric (across all 3 cone types) to that of a 5,000K blackbody, and metameric (for rhodopsin and melanopsin) to that of a 6,000K blackbody. The device may produce an output spectrum (FIG. 11B) for that purpose which achieves a CRI of >80. The component photoreceptor and spectra ratios (relative to photopic luminance) required to achieve a 5,000K (with 6,000K Rod & Melanopsin) biologically directed light stimulation includes: S-cone=M-cone=0.3337; L-cone=0.6663; Rhodopsin=1.0469; Melanopsin=1.0527 with LED1=5.7309; LED2=2.8370; LED3=8.1176; LED4=6.7973; LEDS=5.7251; LED6=0.8824; LED7=6.8239; LED8=6.6527; LED9=7.8776.

The device 300 may be required to apply a color correction for the color variation induced by the difference in melanopsin and rhodopsin excitation, relative to reference cone excitation. The device 300 may produce an output spectrum (FIG. 11C) for that purpose which achieves a CRI of >80. The component photoreceptor and spectra ratios (relative to photopic luminance) required to achieve a 5,000K (with 6,000K Rod & Melanopsin) biologically directed light stimulation ratios with color correction include: S-cone=0.8311; M-cone=0.3337; L-cone=0.6676; Rhodopsin=1.0469; Melanopsin=1.0527 with LED1=6.2245; LED2=4.2179; LED3=5.3482; LED4=5.9605; LEDS=6.0993; LED6=4.5857; LED7=6.1699; LED8=6.2801; LED9=6.2355.

Each distinct spectral component may be associated with electromagnetic radiation emitted by a respective emitter 312 and/or may comprise a subset of the electromagnetic radiation spectrum. The subset of the electromagnetic radiation spectrum may be a discrete continuum of the electromagnetic spectrum.

The active stimulation of all ocular photoreceptor classes by the artificial electromagnetic radiation may be independent active stimulation. The independent active stimulation may comprise a respective channel and/or spectral component of emitted electromagnetic radiation for each respective ocular photoreceptor class.

From the teaching herein, a skilled person is readily able to select a suitable number of emitters 312 such as, one; two; three; four; five; six; seven; eight; nine; ten; or ten or more.

The controllable spectra may comprise narrowband or one or more wideband output including but not limited to 1 nm; 2 nm; 3 nm; 4 nm; 5 nm; 6 nm; 7 nm; 8 nm; 9 nm; nm; 15 nm; 20 nm; 25 nm; 30 nm; 40 nm; 45 nm; 50 nm; 60 nm; 70 nm; 80 nm; 90 nm; 100 nm; or 110 nm.

Each emitter 312 may comprise one or more light source such as, an LED and/or one or more biologically luminescent material. Each emitter 312 may comprise one or more of a solid state light source; an organic luminescent material and/or inorganic luminescent material. Each emitter 312 may comprise a spectrum bandwidth that may either be un-attenuated, or optically attenuated using organic or inorganic substrates to narrow their output spectrum. The one or more emitter may be provided in a combination to control the available modulation ratios of the five classes of human photoreceptors.

Each of the one or more emitter 312 may further comprise one or more filter such as, one or more colored interference filter; one or more spectral filter; and/or one or more neutral density filter for tuning the emitted electromagnetic radiation. The emitted electromagnetic radiation may comprise white light.

The one or more emitter 312, or respective emitters 312 may be independently controllable by the light emitter controller 350. The independent control may be for spectral power manipulation corresponding to a set of photoreceptor class ratios.

The light emitter controller 350 may comprise one or more computer processor. The skilled person will understand from the teaching herein that the controller 350, computer 200 or computer processor 205 may comprise one or more of a microcontroller; a Field-programmable Gate Array (FPGA); or other control generating device. The controller 350, computer 200 or computer processor 205 may be comprised in a single system or distributed systems.

The light emitter controller 350 may utilise a minimisation algorithm (optimisation) to compute spectral outputs based on individual photoreceptor responses. The algorithm may produce high Color Rendering Index (CRI) solutions for all CCTs through the daylight spectrum with variations in rhodopsin and/or melanopsin stimulation. Tests to date show the CRI is better than commercial products (see Table 3).

The light emitter controller 350 may utilise pulsed width modulation (PWM) dimming and/or illumination source measurement.

The method, device 300 or system 400 may further comprise one or more sensor for detecting (the emitted) electromagnetic radiation. The detected electromagnetic radiation may be analysed. The analysis may comprise a spectral analysis. The analysis may be in real-time or close to real-time.

The light emitter controller 350 may comprise one or more processor to determine one or more control value for a desired electromagnetic radiation emission and may control the illumination source to modulate the emitted electromagnetic radiation to the desired emitted electromagnetic radiation. The desired emitted electromagnetic radiation emission may comprise an artificial spectrum or a natural spectrum. The natural spectrum may comprise a solar spectrum. The desired emitted electromagnetic radiation may be to one or more control value for each emitter 312 over time. The one or more control value may be for desired photoreceptor class response(s) which may optionally vary over time. The one or more control value may correct for perceptual color variations caused by color contributions of melanopsin and/or rods (rhodopsin) (FIG. 6).

The method, device 300 or system 400 may generate a spectrum from a desired set of photoreceptor ratios which may comprise ipRGC (melanopsin (i)) and/or rod (rhodopsin (R)) ratios that are calculated from a natural spectrum and cone ratios that are chosen for a given application or preference to generate a perceived color. The perceived color may be selected from a variety of colors such as blue, green, yellow, orange, purple, pink or red for example. For an absolute photopic luminance, a natural spectrum may be defined by the method, device 300 or system 400.

The one or more sensor may measure ambient illumination and/or may receive one or more spectra of daily light exposure. For example, the one or more received spectra may be received from a meteorology bureau or department. The ambient illumination and/or received one or more spectral may allow calculation and provision of supplemental light exposure. That is, the device, method and system of the invention may then generate light spectra absent from the internal environment that are required to achieve the appropriate circadian rhythm by accounting for other ambient lighting. This may find particular application outdoors or indoors where ambient light is provided through windows and/or other existing lighting. An added advantage is that the light can incorporate with existing lighting spectrums which reduces the power usage thereby increasing energy efficiency.

The minimization algorithm may optimize the component spectra contributions to decrease the AUC of the spectra (power). Advantageously, this will provide a most efficient spectra, with minimal wastage of energy relative to photoreceptor excitation, which achieves not merely matching, but good matching.

Advantageously, the correction, through direct changes in the ratio of the cone photoreceptor stimulations, of perceptual variations in hue, saturation and brightness away from a nominal and preferable ‘white’ that are caused by the stimulation of rhodopsin and/or melanopsin is a key advantage that is not present in the prior art.

The light emitter controller 350 may control the one or more emitter 312 to dynamically control melanopsin and rod photoreceptor class activation. The light emitter controller 350 may allow any realisable contrast level (flux) relative to constant (or variable) melanopsin, rod and/or cone activation. The control may comprise real-time or near real-time modulation.

The emitted electromagnetic radiation may comprise a light spectrum that produces photoreceptor class activations that are closer to that resultant from the environmental broadband solar spectrum.

The one or more emitter 312 may comprise a different primary combination to create a white based on the CCT and/or photoreceptor class activation. The illumination may provide a constant CCT while varying rod and/or melanopsin activation. Advantageously, this may correct for changes in color appearance caused by variations in rod and melanopsin activation.

The emitted electromagnetic radiation may comprise a Color Rendering Index (CRI) of at least 85; at least 86; at least 87; at least 88; at least 89; at least 90; at least 91; at least 92; at least 93; at least 94; at least 95. The CRI may be higher than conventional white LEDs and this may be due to a more uniform spectral output. The CRI may comprise a higher Color Rendering Index (CRI) than the Bio Hue Lamp. The CRI may be varied by adjusting the spectral composition of the illumination source 310 or the one or more emitter 312. The CRI may be varied by adjusting the bandwidth and/or dominant wavelength and/or spectral distribution of the one or more emitter 312.

The emitted electromagnetic radiation may stimulate all photoreceptors classes comprising melanopsin containing ipRGCs; rods; cones; and at a system or user defined ration. The emitted electromagnetic radiation may stimulate all ocular photoreceptor proteins comprising: melanopsin; rhodopsin; and opsins. The opsins may comprise three opsins. The three opsins may comprise long wavelength sensitive opsin (erythrolabe) or red opsin; middle wavelength sensitive opsin (chlorolabe) or green opsin; and short wavelength sensitive opsin (cyanolabe) or blue opsin. The stimulation may be independently control the activity level of each of the photoreceptors.

The present invention may provide a Color Rendering Index (CRI) closer to natural lighting (CRI=100) than prior art devices, methods and system.

To mimic the effects of the sun on the photoreceptor excitations, with variation in CCT during the solar day, the following photoreceptor Weber contrast changes, relative to 5,500K, may be required:
Rod contrasts: −27% at low CCT to +14% at high CCT; and
Melanopsin contrasts: −33% at low CCT to +18% at high CCT.

The invention may provide a precise manipulation of visual perception and biological rhythms linked to industry defined human observer functions.

The invention may further provide a broader band spectral distribution to provide a truer representation of the natural environment.

Advantageously, the emitted electromagnetic radiation may not be perceived differently from ambient light (chromaticity invariant) and may be directed to circadian rhythm.

The illumination may be modulated with circadian rhythm. The modulation may be to mimic natural variation in a solar day such as, bluer during the day and warmer (more orange) during the evening. The modulation may be to mimic changes in environmental illumination. The modulation may mimic the natural variation in photoreceptor activations that change during a solar day, while maintaining a single nominated color appearance. The modulation may be different to the natural circadian rhythm. The modulation may be to prepare for travel to a different time zone; to recover from travel from a different time zone or jetlag; to synchronize with work or other activity. The modulation may restore dysfunctional photoreceptor(s) activity in a person with ophthalmic disease to normal (functional) levels.

The device 300, method or system 400 may provide ambient lighting such as, at a home; at a workplace; at a school; at a childcare centre; at a hospital; at a nursing home; at a hotel; at a sleeping quarters; in a transit vehicle; at a road; at a sports field; or any site of human activity including sleep.

The device 300, method and system 400 may be comprised in one or more electronic device such as, a visual display unit or a computer device. The ambient lighting may be provided at a site of a visual display such as, a museum or gallery.

The stimulation or perception may be an animals' stimulation or perception. The animal may be a human; companion animal; a performance animal; or another animal.

Advantageously, the invention can be retrofitted to an existing multi-spectrum light source.

The light emitter controller 350 may comprise the necessary hardware to maintain, and possibly evaluate, reasonably characterised spectral output of the one or more emitter 312, as well as computational resources to determine control values for a desired output.

The controller 350 may provide a reasonably characterised spectral output for a given control value. By reasonably characterised is meant that for known control and component parameters, a known spectral output is generation. One embodiment utilises PWM dimming in conjunction with light source measurements to achieve this. Close-to-real-time spectral analysis either in the physical device, or at a distance, may also be performed.

The controller 350 may operate on the illumination source 310 so that photoreceptor class response values corresponding to a given solar spectrum, or other, artificial or natural spectrum, incorporating user preferences, over time is provided.

The system 350 may determine control values for the one or more emitters 312, corresponding to known spectral components, for desired photoreceptor class responses over time. This may or may not incorporate a method to correct for perceptual color variations caused by color contributions of melanopsin and rods.

The device 300 and system 400 and user interface may be wired, wireless, user-input or hard-wired.

The emitted electromagnetic radiation may comprise a static spectrum, where the spectrum stimulates the photoreceptors in a defined ratio corresponding to [i(Melanopsin) R(od) S M L] using standardised data points (e.g. for house lighting, light therapy in clinical and/or home settings). The emitted electromagnetic radiation may comprise a dynamic spectrum that changes over time, where the light is sufficiently perceptually consistent, but the photoreceptor stimulation ratios change. This may include non-linear transitions between states (e.g. for commercial, medical, hospitality, agricultural, transportation, industrial settings); control and emission systems that modulate biological rhythms (i.e. melanopsin, rods and cone photoreceptor activity) according to characteristic changes in the environmental solar spectrum that will be relevant for phase shifting circadian rhythms after travel, and for correcting differences in regional variations in seasonal light exposure (e.g. relevant for seasonal effective disorders; SAD). This enables light dependent control of mood (alertness). Other applications requiring biologically-directed control of the lighting include animal housing and agriculture.

The invention may be implemented within a visual display such as, a computer screen, a television, or a projector. The device, method or system of the invention can optimise photoreceptor stimulation ratios to improve low-natural-light visibility or reduce light pollution (e.g. street lighting, automotive lighting) and include appropriately and reasonably defined characterised fit-for purpose spectrums, including for human/animal with different photoreceptor spectral responses, including flora.

One possible advantage of the invention's chromaticity correction is that it leads to increased energy efficiency. Australian, and international, lighting standards quantify light output in the visual environment in terms of the L+M cone activation, photometric cd·m⁻² or Lux. This does not provide any estimation of the effects of melanopsin in standard RGB lighting. The melanopsin photoreceptor mediates the perception of brightness, and our chromaticity correction reduces the L+M cone activation, i.e. decreases the cone luminance, cd·m⁻², to account for the increased melanopsin activation, which results in a lower light output and higher energy efficiency with no visible change in brightness or color. The MEF and M/P ratio metrics used in prior art evaluate melanopsin excitation but do not estimate the melanopsin contribution to color or brightness perception.

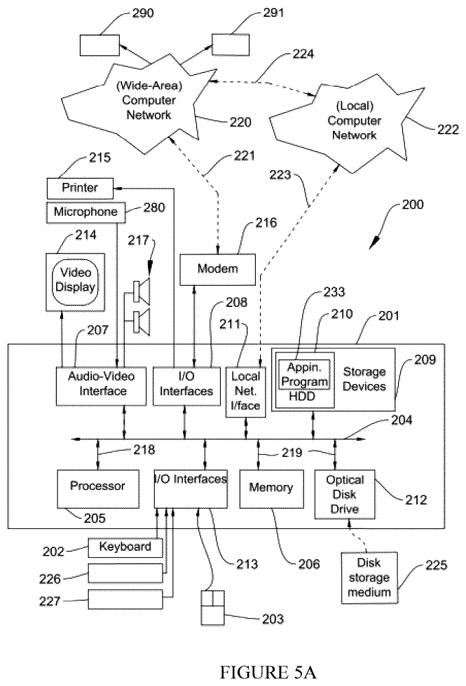

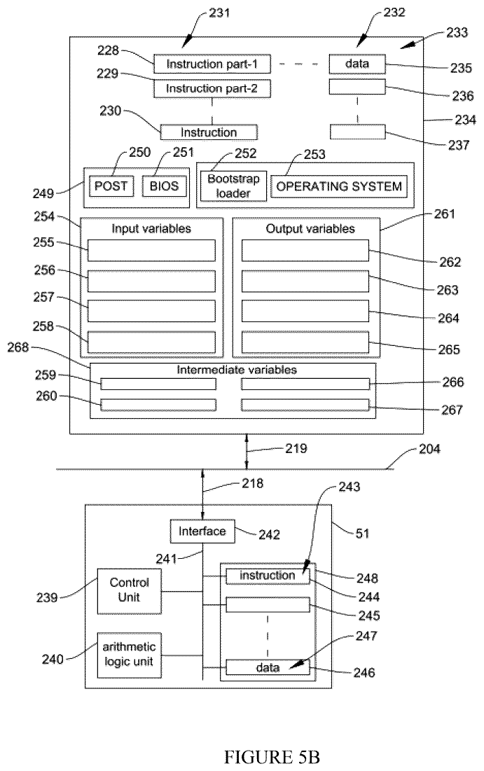

The light emitter controller may take the form of a computer device, such as computer or computer device 200 shown in FIGS. 5A and 5B. In the embodiment shown computer device 200 comprises a computer module 201 comprising input devices such as a keyboard 202, a mouse pointer device 203, a scanner 226, an external hard drive 227, and a microphone 280; and output devices including a printer 215, a display device 214 and loudspeakers 217. In some embodiments video display 214 may comprise a touchscreen.

A Modulator-Demodulator (Modem) transceiver device 216 may be used by the computer module 201 for communicating to and from a communications network 220 via a connection 221. The network 220 may be a wide-area network (WAN), such as the Internet, a cellular telecommunications network, or a private WAN. Through the network 220, computer module 201 may be connected to other similar personal devices 290 or server computers 291. Where the connection 221 is a telephone line, the modem 216 may be a traditional “dial-up” modem. Alternatively, where the connection 221 is a high capacity (e.g.: cable) connection, the modem 216 may be a broadband modem. A wireless modem may also be used for wireless connection to network 220.

The computer module 201 typically includes at least one processor 205, and a memory 206 for example formed from semiconductor random access memory (RAM) and semiconductor read only memory (ROM). The module 201 also includes a number of input/output (I/O) interfaces including: an audio-video interface 207 that couples to the video display 214, loudspeakers 217 and microphone 280; an I/O interface 213 for the keyboard 202, mouse 203, scanner 226 and external hard drive 227; and an interface 208 for the external modem 216 and printer 215. In some implementations, modem 216 may be incorporated within the computer module 201, for example within the interface 208. The computer module 201 also has a local network interface 211 which, via a connection 223, permits coupling of the computer device 200 to a local computer network 222, known as a Local Area Network (LAN).

As also illustrated, the local network 222 may also couple to the wide network 220 via a connection 224, which would typically include a so-called “firewall” device or device of similar functionality. The interface 211 may be formed by an Ethernet circuit card, WiFi, including WiFi HaLow, a Bluetooth wireless arrangement or an IEEE 802.11 wireless arrangement or other suitable interface such as Zigbee and Morse Micro, which may be implemented in (Industrial) Internet of Things ((I)IoT) or home automation technology.

The I/O interfaces 208 and 213 may afford either or both of serial and parallel connectivity, the former typically being implemented according to the Universal Serial Bus (USB) standards and having corresponding USB connectors (not illustrated).

Storage devices 209 are provided and typically include a hard disk drive (HDD) 210. Other storage devices such as, an external HD 227, a disk drive (not shown) and a magnetic tape drive (not shown) may also be used. An optical disk drive 212 is typically provided to act as a non-volatile source of data. Portable memory devices, such as optical disks (e.g.: CD-ROM, DVD, Blu-Ray Disc), USB-RAM, external hard drives and floppy disks for example, may be used as appropriate sources of data to the computer device 200. Another source of data to computer device 200 is provided by the at least one server computer 291 through network 220.

The components 205 to 213 of the computer module 201 typically communicate via an interconnected bus 204 in a manner that results in a conventional mode of operation of computer device 200. In the embodiment shown in FIGS. 5A and 5B, processor 205 is coupled to system bus 204 through connections 218. Similarly, memory 206 and optical disk drive 212 are coupled to the system bus 204 by connections 219. Examples of computer devices 200 on which the described arrangements can be practiced include IBM-PC's and compatibles, Sun Sparc stations, Apple computers; smart phones; tablet computers or like a device comprising a computer module like computer module 201 or (Industrial) Internet of Things ((I)IoT) home automation technology such as, zigbee and morse micro access points and/or connected devices. It is to be understood that when computer device 200 comprises a smart phone or a tablet computer, display device 214 may comprise a touchscreen and other input and output devices may not be included such as, mouse pointer device 203; keyboard 202; scanner 226; and printer 215.

FIG. 2B is a detailed schematic block diagram of processor 205 and a memory 234. The memory 234 represents a logical aggregation of all the memory modules, including the storage device 209 and semiconductor memory 206, which can be accessed by the computer module 201 in FIG. 2A.

The methods of the invention may be implemented using computer device 200 wherein the methods may be implemented as one or more software application programs 233 executable within computer module 201. In particular, the steps of the methods of the invention may be effected by instructions 231 in the software carried out within the computer module 201.

The software instructions 231 may be formed as one or more code modules, each for performing one or more particular tasks. The software 233 may also be divided into two separate parts, in which a first part and the corresponding code modules performs the method of the invention and a second part and the corresponding code modules manage a graphical user interface between the first part and the user.

The software 233 may be stored in a computer readable medium, including in a storage device of a type described herein. The software is loaded into the computer device 200 from the computer readable medium or through network 221 or 223, and then executed by computer device 200. In one example the software 233 is stored on storage medium 225 that is read by optical disk drive 212. Software 233 is typically stored in the HDD 210 or the memory 206.

A computer readable medium having such software 233 or computer program recorded on it is a computer program product. The use of the computer program product in the computer device 200 preferably effects a device or apparatus for implementing the methods of the invention.

In some instances, the software application programs 233 may be supplied to the user encoded on one or more disk storage medium 225 such as a CD-ROM, DVD or Blu-Ray disc, and read via the corresponding drive 212, or alternatively may be read by the user from the networks 220 or 222. Still further, the software can also be loaded into the computer device 200 from other computer readable media. Computer readable storage media refers to any non-transitory tangible storage medium that provides recorded instructions and/or data to the computer module 201 or computer device 200 for execution and/or processing. Examples of such storage media include floppy disks, magnetic tape, CD-ROM, DVD, Blu-ray Disc, a hard disk drive, a ROM or integrated circuit, USB memory, a magneto-optical disk, or a computer readable card such as a PCMCIA card and the like, whether or not such devices are internal or external of the computer module 201. Examples of transitory or non-tangible computer readable transmission media that may also participate in the provision of software application programs 233, instructions 231 and/or data to the computer module 201 include radio or infra-red transmission channels as well as a network connection 221, 223, 334, to another computer or networked device 290, 291 and the Internet or an Intranet including email transmissions and information recorded on Websites and the like.

The second part of the application programs 233 and the corresponding code modules mentioned above may be executed to implement one or more graphical user interfaces (GUIs) to be rendered or otherwise represented upon display 214. Through manipulation of, typically, keyboard 202, mouse 203 and/or screen 214 when comprising a touchscreen, a user of computer device 200 and the methods of the invention may manipulate the interface in a functionally adaptable manner to provide controlling commands and/or input to the applications associated with the GUI(s). Other forms of functionally adaptable user interfaces may also be implemented, such as an audio interface utilizing speech prompts output via loudspeakers 217 and user voice commands input via microphone 280. The manipulations including mouse clicks, screen touches, speech prompts and/or user voice commands may be transmitted via network 220 or 222.

When the computer module 201 is initially powered up, a power-on self-test (POST) program 250 may execute. The POST program 250 is typically stored in a ROM 249 of the semiconductor memory 206. A hardware device such as the ROM 249 is sometimes referred to as firmware. The POST program 250 examines hardware within the computer module 201 to ensure proper functioning, and typically checks processor 205, memory 234 (209, 206), and a basic input-output systems software (BIOS) module 251, also typically stored in ROM 249, for correct operation. Once the POST program 250 has run successfully, BIOS 251 activates hard disk drive 210. Activation of hard disk drive 210 causes a bootstrap loader program 252 that is resident on hard disk drive 210 to execute via processor 205. This loads an operating system 253 into RAM memory 206 upon which operating system 253 commences operation. Operating system 253 is a system level application, executable by processor 205, to fulfil various high level functions, including processor management, memory management, device management, storage management, software application interface, and generic user interface.

Operating system 253 manages memory 234 (209, 206) in order to ensure that each process or application running on computer module 201 has sufficient memory in which to execute without colliding with memory allocated to another process. Furthermore, the different types of memory available in the computer device 200 must be used properly so that each process can run effectively. Accordingly, the aggregated memory 234 is not intended to illustrate how particular segments of memory are allocated, but rather to provide a general view of the memory accessible by computer module 201 and how such is used.

Processor 205 includes a number of functional modules including a control unit 239, an arithmetic logic unit (ALU) 240, and a local or internal memory 248, sometimes called a cache memory. The cache memory 248 typically includes a number of storage registers 244, 245, 246 in a register section storing data 247. One or more internal busses 241 functionally interconnect these functional modules. The processor 205 typically also has one or more interfaces 242 for communicating with external devices via the system bus 204, using a connection 218. The memory 234 is connected to the bus 204 by connection 219.

Application program 233 includes a sequence of instructions 231 that may include conditional branch and loop instructions. Program 233 may also include data 232 which is used in execution of the program 233. The instructions 231 and the data 232 are stored in memory locations 228, 229, 230 and 235, 236, 237, respectively. Depending upon the relative size of the instructions 231 and the memory locations 228-230, a particular instruction may be stored in a single memory location as depicted by the instruction shown in the memory location 230. Alternately, an instruction may be segmented into a number of parts each of which is stored in a separate memory location, as depicted by the instruction segments shown in the memory locations 228 and 229.

In general, processor 205 is given a set of instructions 243 which are executed therein. The processor 205 then waits for a subsequent input, to which processor 205 reacts by executing another set of instructions. Each input may be provided from one or more of a number of sources, including data generated by one or more of the input devices 202, 203, or 214 when comprising a touchscreen, data received from an external source across one of the networks 220, 222, data retrieved from one of the storage devices 206, 209 or data retrieved from a storage medium 225 inserted into the corresponding reader 212. The execution of a set of the instructions may in some cases result in output of data. Execution may also involve storing data or variables to the memory 234.

The disclosed arrangements use input variables 254 that are stored in the memory 234 in corresponding memory locations 255, 256, 257, 258. The described arrangements produce output variables 261 that are stored in the memory 234 in corresponding memory locations 262, 263, 264, 265. Intermediate variables 268 may be stored in memory locations 259, 260, 266 and 267.

The register section 244, 245, 246, the arithmetic logic unit (ALU) 240, and the control unit 239 of the processor 205 work together to perform sequences of micro-operations needed to perform “fetch, decode, and execute” cycles for every instruction in the instruction set making up the program 233. Each fetch, decode, and execute cycle comprises:
a fetch operation, which fetches or reads an instruction 231 from memory location 228, 229, 230;
a decode operation in which control unit 239 determines which instruction has been fetched; and
an execute operation in which the control unit 239 and/or the ALU 240 execute the instruction.

Thereafter, a further fetch, decode, and execute cycle for the next instruction may be executed. Similarly, a store cycle may be performed by which the control unit 239 stores or writes a value to a memory location 232.

Each step or sub-process in the methods of the invention may be associated with one or more segments of the program 233, and may be performed by register section 244-246, the ALU 240, and the control unit 239 in the processor 205 working together to perform the fetch, decode, and execute cycles for every instruction in the instruction set for the noted segments of program 233.

One or more other computers 290 may be connected to the communications network 220 as seen in FIG. 5A. Each such computer 290 may have a similar configuration to the computer module 201 and corresponding peripherals.

One or more other server computer 291 may be connected to the communications network 220. These server computers 291 response to requests from the personal device or other server computers to provide information.

The methods of the invention may alternatively be implemented in dedicated hardware such as one or more integrated circuits performing the functions or sub functions of the described methods. Such dedicated hardware may include graphic processors, digital signal processors, or one or more microprocessors and associated memories.

It will be understood that in order to practice the methods of the invention as described above, it is not necessary that the processors and/or the memories of the processing machine be physically located in the same geographical place. That is, each of the processors and the memories used in the invention may be located in geographically distinct locations and connected so as to communicate in any suitable manner Additionally, it will be understood that each of the processor and/or the memory may be composed of different physical pieces of equipment. Accordingly, it is not necessary that a processor be one single piece of equipment in one location and that the memory be another single piece of equipment in another location. That is, it is contemplated that the processor may be two pieces of equipment in two different physical locations. The two distinct pieces of equipment may be connected in any suitable manner Additionally, the memory may include two or more portions of memory in two or more physical locations.

To explain further, processing as described above is performed by various components and various memories. It will be understood, however, that the processing performed by two distinct components as described above may, in accordance with a further embodiment of the invention be performed by a single component. Further, the processing performed by one distinct component as described above may be performed by two distinct components. In a similar manner, the memory storage performed by two distinct memory portions as described above may, in accordance with a further embodiment of the invention, be performed by a single memory portion. Further, the memory storage performed by one distinct memory portion as described above may be performed by two memory portions.

Further, various technologies may be used to provide communication between the various processors and/or memories, as well as to allow the processors and/or the memories of the invention to communicate with any other entity, i.e., so as to obtain further instructions or to access and use remote memory stores, for example. Such technologies used to provide such communication might include a network, the Internet, Intranet, Extranet, LAN, an Ethernet, a telecommunications network (e.g., a cellular or wireless network) or any client server system that provides communication, for example. Such communications technologies may use any suitable protocol such as TCP/IP, UDP, or OSI, for example.

The skilled person will readily appreciate that the present invention is applicable to current and conventional multi-primary (e.g. RGBw etc) lighting systems. Furthermore, the present invention is independent of the light source and it may accommodate future developments (e.g. organic LEDS, super-luminescent LEDs, lasers).

From the teaching herein, the skilled person will also readily appreciate the invention has applications in ambient lighting e.g. home, industry, transportation—including in extreme regions near the polar-circles when the day-night cycles are different relative to 24-hour day-night cycles at the equator, environmental lighting such as, city and rural settings for flora and fauna, in visual displays such as, TV and cinema, and computer devices such as, laptops, desktop, phones.

The following non-limiting examples illustrate the invention. These examples should not be construed as limiting: the examples are included for the purposes of illustration only. The Examples will be understood to represent an exemplification of the invention.

### Examples

An embodiment of the “biologically-balanced artificial lighting” system was designed, constructed and tested in the QUT Visual Science and Medical Retina Laboratories. The device was used to generate electromagnetic radiation as illustrated in FIGS. 1A; 1B; 2A; 2B; 10; 11 and 12.

Device: The device comprises nine independently-controllable spectra. LED and interference filter combinations achieved narrowband spectra. The individual spectra were homogenised to achieve the emitted light. The individual emitter channels were driven by LED drivers set at 330 mA, and controlled via PWM from a microcontroller that received commands from a custom-designed application. The application performed all necessary calculations and optimisations.

Sensitivity functions used for quantification in this document: The L-, M-, S-cone fundamentals are linear transformations (Smith & Pokorny, 1975) of CIE 1964 supplementary standard color-matching functions. The rhodopsin (rod) spectral sensitivity (V′λ) is the CIE scotopic sensitivity function. The melanopsin-mediated ipRGC excitation (i or I) is computed according to the melanopsin spectral sensitivity function that accounts for cornea and lens spectral filtering (Enezi et al, 2011; Adhikari et al, 2015). Spectral sensitivity functions are normalized with photopic retinal illuminance specified as the sum of L- and M-cone excitations (L+M) (MacLeod Boynton, 1979). For an Equal-Energy-Spectrum (EES) light at 1 photopic Troland (Td) the S-, M-, and L-cone, rod, and melanopsin expressing ipRGC photoreceptor excitations are 0.6667 L-cone Td, 0.3333 M-cone Td, 1 S-cone Td, 1 rod Td, and 1 melanopsin Td. For an EES light, the photoreceptor excitation relative to photopic luminance is l=L/(L+M)=0.6667, m=M/(L+M)=0.3333, s=S/(L+M)=1, r=R/(L+M)=1, i=I/(L+M)=1. An L-cone chromaticity of 0.6667 with an EES light comes about from the CIE XYZ normalization of EES at 1 Td to have x=0.3333, y=0.3333. That CIE Y is normalized as luminance and the luminosity spectral sensitivity has a 2:1 L:M cone contribution (Smith & Pokorny, 1975), when EES is expressed in the MacLeod-Boynton cone excitation space the relative L:M cone weightings at EES are 2:1 (MacLeod & Boynton, 1979).

Scientific evidence (Reference: Altimus et al. 2010; and Dumpala, Zele & Feigl 2019) confirms that the stimulation of all photoreceptors is required to promote the full effect of sleep. Melanopsin expressing ipRGCs, rods and cones all influence visual and non-visual (including circadian) responses.

One advantage of the invention is that, unlike the prior art, the present invention corrects for the changes in the color appearance of the light due to melanopsin and rod activation.

Unlike the prior art which approach artificial lighting from a ‘color theory from light’ perspective which is based on physics, in one embodiment, the present invention provides artificial light as ‘color as a by-product of biological stimulation’, which is based on physiology or physiological response to light.

Another advantage of the present invention is that unlike the prior art, which utilise defined high and/or low states, the present invention is continuous within the limitations of the chosen light spectra and Standard Observer.

Advantageously, the invention may permit spectral outputs to be changed to modulate the rhodopsin and/or melanopsin stimulation (and associated color corrections) in accordance with changes in CCT during solar day, or at a fixed CCT in order to provide the required circadian phase of the user/s with reference to the solar day. This is particularly relevant for geographical locations with extreme seasonal variations in light exposure spectrums and intensity, for international travel, for street lighting, and for agriculture.

Yet another advantage is that parental settings on electronic devices such as, phones, tablet computers etc, may be set so that photoreceptor stimulations are appropriate for the time of day.

Yet another advantage of the invention is that it provides for creation of personalised lighting that permits local variation in the rhodopsin and/or melanopsin stimulation levels such as, in one workspace, while maintaining uniform chromaticity (CCT) within a larger area such as, throughout a building.

In this specification, the terms “comprises”, “comprising” or similar terms are intended to mean a non-exclusive inclusion, such that an apparatus that comprises a list of elements does not include those elements solely, but may well include other elements not listed.

Throughout the specification the aim has been to describe the invention without limiting the invention to any one embodiment or specific collection of features. Persons skilled in the relevant art may realize variations from the specific embodiments that will nonetheless fall within the scope of the invention.

TABLE 1
Coefficient values for each respective ocular photoreceptor
class as defined by a cubic polynomial:

| | a | B | C | d | B |
|---|---|---|---|---|---|
| S | 0.001259126 | −0.034754594 | 0.433092008 | −0.619542968 | 0.075 |
| M | 0.000505513 | −0.010270342 | 0.074854481 | 0.153126403 | 0.025 |
| L | −0.000505513 | 0.010270342 | −0.074854481 | 0.846873597 | 0.025 |
| R | 0.002189895 | −0.049925408 | 0.435812967 | −0.244240435 | 0.05 |
| I | 0.001953407 | −0.047973588 | 0.461696814 | −0.412592279 | 0.05 |

TABLE 2
Photoreceptor class-to-photopic-luminance activation
ratios for CCT values 2,000 to 8,000:

| CCT | S | M | L | R | i |
|---|---|---|---|---|---|
| 2000 | 0.140588689 | 0.262434466 | 0.737565534 | 0.439016579 | 0.333294061 |
| 2010 | 0.142772929 | 0.26297303 | 0.73702697 | 0.441836051 | 0.336195453 |
| 2020 | 0.144968498 | 0.263507069 | 0.736492931 | 0.44464614 | 0.339093033 |
| 2030 | 0.147175217 | 0.264036635 | 0.735963365 | 0.447446831 | 0.341986695 |
| 2040 | 0.149392909 | 0.26456178 | 0.73543822 | 0.450238115 | 0.344876335 |
| 2050 | 0.151621396 | 0.265082556 | 0.734917444 | 0.453019981 | 0.34776185 |
| 2060 | 0.153860502 | 0.265599015 | 0.734400985 | 0.455792419 | 0.350643142 |
| 2070 | 0.156110053 | 0.266111206 | 0.733888794 | 0.458555424 | 0.353520114 |
| 2080 | 0.158369873 | 0.266619179 | 0.733380821 | 0.46130899 | 0.356392672 |
| 2090 | 0.16063979 | 0.267122983 | 0.732877017 | 0.464053112 | 0.359260725 |
| 2100 | 0.162919632 | 0.267622666 | 0.732377334 | 0.466787788 | 0.362124183 |
| 2110 | 0.165209227 | 0.268118275 | 0.731881725 | 0.469513016 | 0.364982961 |
| 2120 | 0.167508405 | 0.268609857 | 0.731390143 | 0.472228795 | 0.367836974 |
| 2130 | 0.169816997 | 0.269097459 | 0.730902541 | 0.474935126 | 0.37068614 |
| 2140 | 0.172134835 | 0.269581125 | 0.730418875 | 0.477632012 | 0.37353038 |
| 2150 | 0.174461751 | 0.270060902 | 0.729939098 | 0.480319454 | 0.376369616 |
| 2160 | 0.176797581 | 0.270536832 | 0.729463168 | 0.482997459 | 0.379203774 |
| 2170 | 0.17914216 | 0.271008959 | 0.728991041 | 0.48566603 | 0.382032779 |
| 2180 | 0.181495323 | 0.271477327 | 0.728522673 | 0.488325174 | 0.384856562 |
| 2190 | 0.18385691 | 0.271941978 | 0.728058022 | 0.490974899 | 0.387675054 |
| 2200 | 0.186226758 | 0.272402953 | 0.727597047 | 0.493615213 | 0.390488187 |
| 2210 | 0.188604709 | 0.272860295 | 0.727139705 | 0.496246124 | 0.393295897 |
| 2220 | 0.190990603 | 0.273314042 | 0.726685958 | 0.498867643 | 0.396098121 |
| 2230 | 0.193384284 | 0.273764236 | 0.726235764 | 0.501479781 | 0.398894798 |
| 2240 | 0.195785595 | 0.274210916 | 0.725789084 | 0.50408255 | 0.401685869 |
| 2250 | 0.198194381 | 0.274654121 | 0.725345879 | 0.506675962 | 0.404471276 |
| 2260 | 0.20061049 | 0.275093889 | 0.724906111 | 0.509260031 | 0.407250964 |
| 2270 | 0.203033768 | 0.275530258 | 0.724469742 | 0.511834771 | 0.410024879 |
| 2280 | 0.205464065 | 0.275963266 | 0.724036734 | 0.514400197 | 0.41279297 |
| 2290 | 0.207901232 | 0.27639295 | 0.72360705 | 0.516956324 | 0.415555185 |
| 2300 | 0.210345121 | 0.276819345 | 0.723180655 | 0.519503169 | 0.418311476 |
| 2310 | 0.212795583 | 0.277242487 | 0.722757513 | 0.522040749 | 0.421061795 |
| 2320 | 0.215252474 | 0.277662413 | 0.722337587 | 0.524569082 | 0.423806098 |
| 2330 | 0.21771565 | 0.278079156 | 0.721920844 | 0.527088185 | 0.42654434 |
| 2340 | 0.220184968 | 0.278492752 | 0.721507248 | 0.529598078 | 0.429276478 |
| 2350 | 0.222660285 | 0.278903233 | 0.721096767 | 0.53209878 | 0.432002472 |
| 2360 | 0.225141463 | 0.279310634 | 0.720689366 | 0.53459031 | 0.434722282 |
| 2370 | 0.227628362 | 0.279714988 | 0.720285012 | 0.537072691 | 0.437435869 |
| 2380 | 0.230120844 | 0.280116326 | 0.719883674 | 0.539545942 | 0.440143198 |
| 2390 | 0.232618773 | 0.280514682 | 0.719485318 | 0.542010085 | 0.442844232 |
| 2400 | 0.235122015 | 0.280910087 | 0.719089913 | 0.544465142 | 0.445538937 |
| 2410 | 0.237630437 | 0.281302571 | 0.718697429 | 0.546911135 | 0.448227281 |
| 2420 | 0.240143905 | 0.281692167 | 0.718307833 | 0.549348089 | 0.450909232 |
| 2430 | 0.242662289 | 0.282078904 | 0.717921096 | 0.551776025 | 0.453584759 |
| 2440 | 0.24518546 | 0.282462813 | 0.717537187 | 0.554194968 | 0.456253834 |
| 2450 | 0.24771329 | 0.282843922 | 0.717156078 | 0.556604941 | 0.458916429 |
| 2460 | 0.250245652 | 0.283222262 | 0.716777738 | 0.55900597 | 0.461572516 |
| 2470 | 0.25278242 | 0.283597861 | 0.716402139 | 0.56139808 | 0.464222071 |
| 2480 | 0.255323471 | 0.283970747 | 0.716029253 | 0.563781294 | 0.466865069 |
| 2490 | 0.257868682 | 0.284340948 | 0.715659052 | 0.56615564 | 0.469501486 |
| 2500 | 0.260417931 | 0.284708493 | 0.715291507 | 0.568521143 | 0.472131301 |
| 2510 | 0.262971098 | 0.285073409 | 0.714926591 | 0.570877829 | 0.474754491 |
| 2520 | 0.265528064 | 0.285435722 | 0.714564278 | 0.573225725 | 0.477371037 |
| 2530 | 0.268088713 | 0.285795459 | 0.714204541 | 0.575564858 | 0.479980919 |
| 2540 | 0.270652927 | 0.286152647 | 0.713847353 | 0.577895254 | 0.482584119 |
| 2550 | 0.273220592 | 0.28650731 | 0.71349269 | 0.580216941 | 0.48518062 |
| 2560 | 0.275791595 | 0.286859476 | 0.713140524 | 0.582529946 | 0.487770405 |
| 2570 | 0.278365823 | 0.287209169 | 0.712790831 | 0.584834298 | 0.490353459 |
| 2580 | 0.280943165 | 0.287556415 | 0.712443585 | 0.587130025 | 0.492929767 |
| 2590 | 0.283523511 | 0.287901236 | 0.712098764 | 0.589417153 | 0.495499316 |
| 2600 | 0.286106754 | 0.288243659 | 0.711756341 | 0.591695713 | 0.498062093 |
| 2610 | 0.288692785 | 0.288583707 | 0.711416293 | 0.593965733 | 0.500618084 |
| 2620 | 0.291281499 | 0.288921404 | 0.711078596 | 0.596227241 | 0.503167281 |
| 2630 | 0.293872791 | 0.289256772 | 0.710743228 | 0.598480266 | 0.505709671 |
| 2640 | 0.296466558 | 0.289589836 | 0.710410164 | 0.600724839 | 0.508245245 |
| 2650 | 0.299062697 | 0.289920617 | 0.710079383 | 0.602960987 | 0.510773995 |
| 2660 | 0.301661108 | 0.290249139 | 0.709750861 | 0.60518874 | 0.513295912 |
| 2670 | 0.30426169 | 0.290575423 | 0.709424577 | 0.607408129 | 0.515810989 |
| 2680 | 0.306864345 | 0.290899492 | 0.709100508 | 0.609619182 | 0.518319219 |
| 2690 | 0.309468976 | 0.291221367 | 0.708778633 | 0.611821929 | 0.520820596 |
| 2700 | 0.312075487 | 0.291541069 | 0.708458931 | 0.614016401 | 0.523315114 |
| 2710 | 0.314683782 | 0.29185862 | 0.70814138 | 0.616202628 | 0.52580277 |
| 2720 | 0.317293767 | 0.29217404 | 0.70782596 | 0.618380639 | 0.528283558 |
| 2730 | 0.319905351 | 0.29248735 | 0.70751265 | 0.620550465 | 0.530757476 |
| 2740 | 0.322518441 | 0.29279857 | 0.70720143 | 0.622712137 | 0.533224521 |
| 2750 | 0.325132947 | 0.29310772 | 0.70689228 | 0.624865684 | 0.53568469 |
| 2760 | 0.32774878 | 0.293414821 | 0.706585179 | 0.627011137 | 0.538137982 |
| 2770 | 0.330365852 | 0.293719891 | 0.706280109 | 0.629148527 | 0.540584396 |
| 2780 | 0.332984075 | 0.29402295 | 0.70597705 | 0.631277883 | 0.543023931 |
| 2790 | 0.335603364 | 0.294324017 | 0.705675983 | 0.633399238 | 0.545456588 |
| 2800 | 0.338223634 | 0.294623111 | 0.705376889 | 0.635512621 | 0.547882366 |
| 2810 | 0.340844801 | 0.29492025 | 0.70507975 | 0.637618064 | 0.550301268 |
| 2820 | 0.343466783 | 0.295215454 | 0.704784546 | 0.639715596 | 0.552713294 |
| 2830 | 0.346089498 | 0.295508739 | 0.704491261 | 0.641805249 | 0.555118447 |
| 2840 | 0.348712865 | 0.295800125 | 0.704199875 | 0.643887054 | 0.557516729 |
| 2850 | 0.351336806 | 0.296089628 | 0.703910372 | 0.645961041 | 0.559908144 |
| 2860 | 0.353961241 | 0.296377266 | 0.703622734 | 0.648027242 | 0.562292695 |
| 2870 | 0.356586094 | 0.296663057 | 0.703336943 | 0.650085686 | 0.564670386 |
| 2880 | 0.359211288 | 0.296947017 | 0.703052983 | 0.652136405 | 0.567041221 |
| 2890 | 0.361836748 | 0.297229164 | 0.702770836 | 0.654179431 | 0.569405205 |
| 2900 | 0.364462399 | 0.297509514 | 0.702490486 | 0.656214792 | 0.571762343 |
| 2910 | 0.367088168 | 0.297788083 | 0.702211917 | 0.658242522 | 0.574112642 |
| 2920 | 0.369713982 | 0.298064889 | 0.701935111 | 0.660262649 | 0.576456107 |
| 2930 | 0.372339771 | 0.298339946 | 0.701660054 | 0.662275206 | 0.578792744 |
| 2940 | 0.374965465 | 0.298613272 | 0.701386728 | 0.664280222 | 0.58112256 |
| 2950 | 0.377590992 | 0.298884881 | 0.701115119 | 0.66627773 | 0.583445563 |
| 2960 | 0.380216286 | 0.299154789 | 0.700845211 | 0.668267758 | 0.585761759 |
| 2970 | 0.382841277 | 0.299423011 | 0.700576989 | 0.670250339 | 0.588071158 |
| 2980 | 0.385465901 | 0.299689563 | 0.700310437 | 0.672225503 | 0.590373767 |
| 2990 | 0.388090091 | 0.29995446 | 0.70004554 | 0.674193279 | 0.592669594 |
| 3000 | 0.390713782 | 0.300217716 | 0.699782284 | 0.6761537 | 0.594958648 |
| 3010 | 0.393336911 | 0.300479346 | 0.699520654 | 0.678106796 | 0.597240939 |
| 3020 | 0.395959414 | 0.300739364 | 0.699260636 | 0.680052596 | 0.599516476 |
| 3030 | 0.398581229 | 0.300997786 | 0.699002214 | 0.681991132 | 0.601785269 |
| 3040 | 0.401202295 | 0.301254624 | 0.698745376 | 0.683922434 | 0.604047327 |
| 3050 | 0.403822552 | 0.301509893 | 0.698490107 | 0.685846532 | 0.606302662 |
| 3060 | 0.40644194 | 0.301763606 | 0.698236394 | 0.687763457 | 0.608551283 |
| 3070 | 0.4090604 | 0.302015778 | 0.697984222 | 0.689673239 | 0.610793202 |
| 3080 | 0.411677875 | 0.302266422 | 0.697733578 | 0.691575907 | 0.613028429 |
| 3090 | 0.414294308 | 0.30251555 | 0.69748445 | 0.693471493 | 0.615256977 |
| 3100 | 0.416909641 | 0.302763177 | 0.697236823 | 0.695360026 | 0.617478856 |
| 3110 | 0.419523821 | 0.303009316 | 0.696990684 | 0.697241535 | 0.619694079 |
| 3120 | 0.422136792 | 0.303253978 | 0.696746022 | 0.699116052 | 0.621902657 |
| 3130 | 0.424748501 | 0.303497177 | 0.696502823 | 0.700983606 | 0.624104603 |
| 3140 | 0.427358894 | 0.303738926 | 0.696261074 | 0.702844226 | 0.626299929 |
| 3150 | 0.42996792 | 0.303979237 | 0.696020763 | 0.704697942 | 0.628488649 |
| 3160 | 0.432575526 | 0.304218122 | 0.695781878 | 0.706544784 | 0.630670774 |
| 3170 | 0.435181663 | 0.304455593 | 0.695544407 | 0.708384782 | 0.632846319 |
| 3180 | 0.43778628 | 0.304691663 | 0.695308337 | 0.710217964 | 0.635015296 |
| 3190 | 0.440389327 | 0.304926344 | 0.695073656 | 0.71204436 | 0.637177719 |
| 3200 | 0.442990758 | 0.305159646 | 0.694840354 | 0.713864 | 0.639333601 |
| 3210 | 0.445590523 | 0.305391582 | 0.694608418 | 0.715676913 | 0.641482957 |
| 3220 | 0.448188575 | 0.305622163 | 0.694377837 | 0.717483127 | 0.6436258 |
| 3230 | 0.450784869 | 0.305851401 | 0.694148599 | 0.719282672 | 0.645762145 |
| 3240 | 0.453379359 | 0.306079307 | 0.693920693 | 0.721075576 | 0.647892006 |
| 3250 | 0.455972 | 0.306305892 | 0.693694108 | 0.722861869 | 0.650015398 |
| 3260 | 0.458562747 | 0.306531167 | 0.693468833 | 0.724641579 | 0.652132334 |
| 3270 | 0.461151557 | 0.306755142 | 0.693244858 | 0.726414735 | 0.654242831 |
| 3280 | 0.463738387 | 0.306977829 | 0.693022171 | 0.728181366 | 0.656346902 |
| 3290 | 0.466323195 | 0.307199239 | 0.692800761 | 0.729941499 | 0.658444564 |
| 3300 | 0.46890594 | 0.307419382 | 0.692580618 | 0.731695164 | 0.66053583 |
| 3310 | 0.471486579 | 0.307638267 | 0.692361733 | 0.733442388 | 0.662620717 |
| 3320 | 0.474065074 | 0.307855906 | 0.692144094 | 0.735183199 | 0.66469924 |
| 3330 | 0.476641384 | 0.308072309 | 0.691927691 | 0.736917627 | 0.666771415 |
| 3340 | 0.47921547 | 0.308287486 | 0.691712514 | 0.738645698 | 0.668837256 |
| 3350 | 0.481787294 | 0.308501447 | 0.691498553 | 0.74036744 | 0.670896781 |
| 3360 | 0.484356818 | 0.308714201 | 0.691285799 | 0.742082882 | 0.672950004 |
| 3370 | 0.486924005 | 0.308925759 | 0.691074241 | 0.743792051 | 0.674996943 |
| 3380 | 0.489488819 | 0.30913613 | 0.69086387 | 0.745494974 | 0.677037612 |
| 3390 | 0.492051222 | 0.309345323 | 0.690654677 | 0.747191679 | 0.679072028 |
| 3400 | 0.49461118 | 0.309553349 | 0.690446651 | 0.748882194 | 0.681100208 |
| 3410 | 0.497168658 | 0.309760216 | 0.690239784 | 0.750566545 | 0.683122167 |
| 3420 | 0.499723621 | 0.309965934 | 0.690034066 | 0.75224476 | 0.685137923 |
| 3430 | 0.502276036 | 0.310170512 | 0.689829488 | 0.753916866 | 0.687147492 |
| 3440 | 0.50482587 | 0.310373958 | 0.689626042 | 0.755582889 | 0.68915089 |
| 3450 | 0.50737309 | 0.310576283 | 0.689423717 | 0.757242857 | 0.691148133 |
| 3460 | 0.509917663 | 0.310777494 | 0.689222506 | 0.758896796 | 0.69313924 |
| 3470 | 0.512459559 | 0.310977601 | 0.689022399 | 0.760544732 | 0.695124226 |
| 3480 | 0.514998746 | 0.311176612 | 0.688823388 | 0.762186694 | 0.697103109 |
| 3490 | 0.517535193 | 0.311374536 | 0.688625464 | 0.763822705 | 0.699075905 |
| 3500 | 0.520068871 | 0.311571381 | 0.688428619 | 0.765452794 | 0.701042632 |
| 3510 | 0.522599751 | 0.311767157 | 0.688232843 | 0.767076986 | 0.703003306 |
| 3520 | 0.525127802 | 0.31196187 | 0.68803813 | 0.768695308 | 0.704957944 |
| 3530 | 0.527652997 | 0.31215553 | 0.68784447 | 0.770307784 | 0.706906565 |
| 3540 | 0.530175308 | 0.312348144 | 0.687651856 | 0.771914442 | 0.708849184 |
| 3550 | 0.532694707 | 0.312539721 | 0.687460279 | 0.773515307 | 0.71078582 |
| 3560 | 0.535211166 | 0.312730268 | 0.687269732 | 0.775110404 | 0.712716489 |
| 3570 | 0.537724661 | 0.312919794 | 0.687080206 | 0.776699759 | 0.714641209 |
| 3580 | 0.540235163 | 0.313108306 | 0.686891694 | 0.778283397 | 0.716559997 |
| 3590 | 0.542742649 | 0.313295813 | 0.686704187 | 0.779861344 | 0.718472871 |
| 3600 | 0.545247091 | 0.313482321 | 0.686517679 | 0.781433625 | 0.720379848 |
| 3610 | 0.547748467 | 0.313667839 | 0.686332161 | 0.783000265 | 0.722280945 |
| 3620 | 0.550246751 | 0.313852374 | 0.686147626 | 0.784561289 | 0.724176181 |
| 3630 | 0.552741919 | 0.314035933 | 0.685964067 | 0.786116722 | 0.726065572 |
| 3640 | 0.555233949 | 0.314218524 | 0.685781476 | 0.787666588 | 0.727949136 |
| 3650 | 0.557722816 | 0.314400154 | 0.685599846 | 0.789210913 | 0.729826891 |
| 3660 | 0.5602085 | 0.31458083 | 0.68541917 | 0.790749721 | 0.731698854 |
| 3670 | 0.562690976 | 0.31476056 | 0.68523944 | 0.792283035 | 0.733565043 |
| 3680 | 0.565170224 | 0.31493935 | 0.68506065 | 0.793810882 | 0.735425476 |
| 3690 | 0.567646223 | 0.315117208 | 0.684882792 | 0.795333284 | 0.737280169 |
| 3700 | 0.57011895 | 0.31529414 | 0.68470586 | 0.796850266 | 0.739129142 |
| 3710 | 0.572588387 | 0.315470154 | 0.684529846 | 0.798361852 | 0.740972411 |
| 3720 | 0.575054512 | 0.315645256 | 0.684354744 | 0.799868065 | 0.742809995 |
| 3730 | 0.577517306 | 0.315819453 | 0.684180547 | 0.801368931 | 0.74464191 |
| 3740 | 0.579976749 | 0.315992751 | 0.684007249 | 0.802864471 | 0.746468175 |
| 3750 | 0.582432822 | 0.316165158 | 0.683834842 | 0.80435471 | 0.748288807 |
| 3760 | 0.584885508 | 0.316336679 | 0.683663321 | 0.805839671 | 0.750103824 |
| 3770 | 0.587334786 | 0.316507322 | 0.683492678 | 0.807319378 | 0.751913243 |
| 3780 | 0.58978064 | 0.316677093 | 0.683322907 | 0.808793854 | 0.753717083 |
| 3790 | 0.592223053 | 0.316845998 | 0.683154002 | 0.810263122 | 0.755515362 |
| 3800 | 0.594662006 | 0.317014043 | 0.682985957 | 0.811727204 | 0.757308095 |
| 3810 | 0.597097483 | 0.317181234 | 0.682818766 | 0.813186125 | 0.759095303 |
| 3820 | 0.599529467 | 0.317347579 | 0.682652421 | 0.814639906 | 0.760877001 |
| 3830 | 0.601957942 | 0.317513082 | 0.682486918 | 0.81608857 | 0.762653209 |
| 3840 | 0.604382893 | 0.31767775 | 0.68232225 | 0.817532141 | 0.764423942 |
| 3850 | 0.606804304 | 0.317841589 | 0.682158411 | 0.818970639 | 0.76618922 |
| 3860 | 0.609222159 | 0.318004606 | 0.681995394 | 0.820404089 | 0.76794906 |
| 3870 | 0.611636444 | 0.318166805 | 0.681833195 | 0.821832512 | 0.769703479 |
| 3880 | 0.614047145 | 0.318328192 | 0.681671808 | 0.82325593 | 0.771452495 |
| 3890 | 0.616454246 | 0.318488774 | 0.681511226 | 0.824674365 | 0.773196126 |
| 3900 | 0.618857734 | 0.318648557 | 0.681351443 | 0.82608784 | 0.77493439 |
| 3910 | 0.621257597 | 0.318807545 | 0.681192455 | 0.827496375 | 0.776667303 |
| 3920 | 0.623653819 | 0.318965744 | 0.681034256 | 0.828899994 | 0.778394883 |
| 3930 | 0.626046388 | 0.319123161 | 0.680876839 | 0.830298717 | 0.780117149 |
| 3940 | 0.628435292 | 0.3192798 | 0.6807202 | 0.831692566 | 0.781834116 |
| 3950 | 0.630820518 | 0.319435667 | 0.680564333 | 0.833081563 | 0.783545804 |
| 3960 | 0.633202054 | 0.319590767 | 0.680409233 | 0.834465728 | 0.78525223 |
| 3970 | 0.635579888 | 0.319745107 | 0.680254893 | 0.835845084 | 0.78695341 |
| 3980 | 0.637954008 | 0.31989869 | 0.68010131 | 0.837219651 | 0.788649363 |
| 3990 | 0.640324404 | 0.320051523 | 0.679948477 | 0.83858945 | 0.790340105 |
| 4000 | 0.642691064 | 0.32020361 | 0.67979639 | 0.839954502 | 0.792025655 |
| 4010 | 0.645053977 | 0.320354957 | 0.679645043 | 0.841314828 | 0.793706029 |
| 4020 | 0.647413133 | 0.320505569 | 0.679494431 | 0.842670449 | 0.795381245 |
| 4030 | 0.649768521 | 0.320655451 | 0.679344549 | 0.844021385 | 0.79705132 |
| 4040 | 0.652120133 | 0.320804607 | 0.679195393 | 0.845367657 | 0.798716272 |
| 4050 | 0.654467957 | 0.320953044 | 0.679046956 | 0.846709285 | 0.800376117 |
| 4060 | 0.656811985 | 0.321100766 | 0.678899234 | 0.84804629 | 0.802030873 |
| 4070 | 0.659152207 | 0.321247777 | 0.678752223 | 0.849378692 | 0.803680558 |
| 4080 | 0.661488615 | 0.321394083 | 0.678605917 | 0.850706511 | 0.805325187 |
| 4090 | 0.663821199 | 0.321539688 | 0.678460312 | 0.852029766 | 0.806964779 |
| 4100 | 0.666149951 | 0.321684598 | 0.678315402 | 0.853348479 | 0.808599351 |
| 4110 | 0.668474863 | 0.321828816 | 0.678171184 | 0.854662668 | 0.810228919 |
| 4120 | 0.670795926 | 0.321972348 | 0.678027652 | 0.855972354 | 0.811853501 |
| 4130 | 0.673113134 | 0.322115199 | 0.677884801 | 0.857277556 | 0.813473113 |
| 4140 | 0.675426478 | 0.322257372 | 0.677742628 | 0.858578293 | 0.815087773 |
| 4150 | 0.677735951 | 0.322398872 | 0.677601128 | 0.859874586 | 0.816697497 |
| 4160 | 0.680041546 | 0.322539705 | 0.677460295 | 0.861166453 | 0.818302303 |
| 4170 | 0.682343257 | 0.322679873 | 0.677320127 | 0.862453914 | 0.819902207 |
| 4180 | 0.684641075 | 0.322819383 | 0.677180617 | 0.863736988 | 0.821497226 |
| 4190 | 0.686934995 | 0.322958237 | 0.677041763 | 0.865015693 | 0.823087377 |
| 4200 | 0.689225011 | 0.323096441 | 0.676903559 | 0.86629005 | 0.824672676 |
| 4210 | 0.691511117 | 0.323233999 | 0.676766001 | 0.867560076 | 0.826253141 |
| 4220 | 0.693793306 | 0.323370915 | 0.676629085 | 0.868825791 | 0.827828788 |
| 4230 | 0.696071573 | 0.323507194 | 0.676492806 | 0.870087213 | 0.829399633 |
| 4240 | 0.698345912 | 0.323642839 | 0.676357161 | 0.871344361 | 0.830965693 |
| 4250 | 0.700616319 | 0.323777855 | 0.676222145 | 0.872597253 | 0.832526986 |
| 4260 | 0.702882788 | 0.323912245 | 0.676087755 | 0.873845908 | 0.834083526 |
| 4270 | 0.705145314 | 0.324046015 | 0.675953985 | 0.875090344 | 0.835635331 |
| 4280 | 0.707403893 | 0.324179168 | 0.675820832 | 0.87633058 | 0.837182417 |
| 4290 | 0.70965852 | 0.324311708 | 0.675688292 | 0.877566633 | 0.838724801 |
| 4300 | 0.71190919 | 0.324443638 | 0.675556362 | 0.878798521 | 0.840262498 |
| 4310 | 0.7141559 | 0.324574964 | 0.675425036 | 0.880026263 | 0.841795526 |
| 4320 | 0.716398645 | 0.324705689 | 0.675294311 | 0.881249877 | 0.8433239 |
| 4330 | 0.718637422 | 0.324835816 | 0.675164184 | 0.882469379 | 0.844847636 |
| 4340 | 0.720872227 | 0.324965351 | 0.675034649 | 0.883684789 | 0.846366752 |
| 4350 | 0.723103057 | 0.325094295 | 0.674905705 | 0.884896123 | 0.847881262 |
| 4360 | 0.725329908 | 0.325222655 | 0.674777345 | 0.886103399 | 0.849391184 |
| 4370 | 0.727552778 | 0.325350432 | 0.674649568 | 0.887306634 | 0.850896532 |
| 4380 | 0.729771662 | 0.325477631 | 0.674522369 | 0.888505846 | 0.852397324 |
| 4390 | 0.731986559 | 0.325604255 | 0.674395745 | 0.889701052 | 0.853893574 |
| 4400 | 0.734197466 | 0.325730308 | 0.674269692 | 0.89089227 | 0.855385299 |
| 4410 | 0.73640438 | 0.325855795 | 0.674144205 | 0.892079516 | 0.856872516 |
| 4420 | 0.738607299 | 0.325980717 | 0.674019283 | 0.893262807 | 0.858355239 |
| 4430 | 0.74080622 | 0.32610508 | 0.67389492 | 0.89444216 | 0.859833484 |
| 4440 | 0.743001143 | 0.326228886 | 0.673771114 | 0.895617593 | 0.861307267 |
| 4450 | 0.745192064 | 0.326352139 | 0.673647861 | 0.896789121 | 0.862776604 |
| 4460 | 0.747378982 | 0.326474843 | 0.673525157 | 0.897956762 | 0.864241511 |
| 4470 | 0.749561896 | 0.326597001 | 0.673402999 | 0.899120532 | 0.865702003 |
| 4480 | 0.751740804 | 0.326718616 | 0.673281384 | 0.900280448 | 0.867158095 |
| 4490 | 0.753915704 | 0.326839692 | 0.673160308 | 0.901436525 | 0.868609803 |
| 4500 | 0.756086596 | 0.326960232 | 0.673039768 | 0.902588782 | 0.870057143 |
| 4510 | 0.758253478 | 0.327080239 | 0.672919761 | 0.903737232 | 0.87150013 |
| 4520 | 0.76041635 | 0.327199718 | 0.672800282 | 0.904881894 | 0.872938779 |
| 4530 | 0.762575211 | 0.32731867 | 0.67268133 | 0.906022783 | 0.874373106 |
| 4540 | 0.76473006 | 0.3274371 | 0.6725629 | 0.907159914 | 0.875803125 |
| 4550 | 0.766880897 | 0.32755501 | 0.67244499 | 0.908293305 | 0.877228853 |
| 4560 | 0.769027721 | 0.327672405 | 0.672327595 | 0.90942297 | 0.878650304 |
| 4570 | 0.771170532 | 0.327789286 | 0.672210714 | 0.910548926 | 0.880067494 |
| 4580 | 0.773309329 | 0.327905657 | 0.672094343 | 0.911671188 | 0.881480437 |
| 4590 | 0.775444114 | 0.328021522 | 0.671978478 | 0.912789772 | 0.882889148 |
| 4600 | 0.777574885 | 0.328136883 | 0.671863117 | 0.913904694 | 0.884293644 |
| 4610 | 0.779701644 | 0.328251744 | 0.671748256 | 0.915015968 | 0.885693937 |
| 4620 | 0.78182439 | 0.328366107 | 0.671633893 | 0.916123611 | 0.887090044 |
| 4630 | 0.783943124 | 0.328479976 | 0.671520024 | 0.917227637 | 0.888481979 |
| 4640 | 0.786057846 | 0.328593353 | 0.671406647 | 0.918328063 | 0.889869757 |
| 4650 | 0.788168558 | 0.328706242 | 0.671293758 | 0.919424902 | 0.891253393 |
| 4660 | 0.790275259 | 0.328818645 | 0.671181355 | 0.920518171 | 0.892632901 |
| 4670 | 0.792377952 | 0.328930566 | 0.671069434 | 0.921607883 | 0.894008297 |
| 4680 | 0.794476636 | 0.329042008 | 0.670957992 | 0.922694055 | 0.895379594 |
| 4690 | 0.796571313 | 0.329152972 | 0.670847028 | 0.923776701 | 0.896746807 |
| 4700 | 0.798661984 | 0.329263463 | 0.670736537 | 0.924855836 | 0.898109951 |
| 4710 | 0.800748651 | 0.329373483 | 0.670626517 | 0.925931474 | 0.89946904 |
| 4720 | 0.802831314 | 0.329483034 | 0.670516966 | 0.92700363 | 0.900824089 |
| 4730 | 0.804909976 | 0.329592121 | 0.670407879 | 0.92807232 | 0.902175112 |
| 4740 | 0.806984638 | 0.329700744 | 0.670299256 | 0.929137556 | 0.903522123 |
| 4750 | 0.809055302 | 0.329808908 | 0.670191092 | 0.930199354 | 0.904865137 |
| 4760 | 0.811121969 | 0.329916614 | 0.670083386 | 0.931257728 | 0.906204167 |
| 4770 | 0.813184641 | 0.330023867 | 0.669976133 | 0.932312693 | 0.907539229 |
| 4780 | 0.815243321 | 0.330130667 | 0.669869333 | 0.933364262 | 0.908870336 |
| 4790 | 0.81729801 | 0.330237019 | 0.669762981 | 0.93441245 | 0.910197501 |
| 4800 | 0.819348711 | 0.330342924 | 0.669657076 | 0.935457271 | 0.91152074 |
| 4810 | 0.821395426 | 0.330448385 | 0.669551615 | 0.936498738 | 0.912840067 |
| 4820 | 0.823438157 | 0.330553405 | 0.669446595 | 0.937536866 | 0.914155494 |
| 4830 | 0.825476906 | 0.330657987 | 0.669342013 | 0.938571669 | 0.915467036 |
| 4840 | 0.827511677 | 0.330762133 | 0.669237867 | 0.939603161 | 0.916774707 |
| 4850 | 0.829542471 | 0.330865845 | 0.669134155 | 0.940631355 | 0.918078521 |
| 4860 | 0.831569292 | 0.330969127 | 0.669030873 | 0.941656264 | 0.919378491 |
| 4870 | 0.833592141 | 0.33107198 | 0.66892802 | 0.942677903 | 0.920674631 |
| 4880 | 0.835611023 | 0.331174407 | 0.668825593 | 0.943696285 | 0.921966955 |
| 4890 | 0.83762594 | 0.331276411 | 0.668723589 | 0.944711424 | 0.923255476 |
| 4900 | 0.839636894 | 0.331377993 | 0.668622007 | 0.945723332 | 0.924540208 |
| 4910 | 0.84164389 | 0.331479158 | 0.668520842 | 0.946732024 | 0.925821164 |
| 4920 | 0.843646929 | 0.331579906 | 0.668420094 | 0.947737512 | 0.927098358 |
| 4930 | 0.845646016 | 0.33168024 | 0.66831976 | 0.94873981 | 0.928371803 |
| 4940 | 0.847641153 | 0.331780164 | 0.668219836 | 0.949738931 | 0.929641513 |
| 4950 | 0.849632344 | 0.331879678 | 0.668120322 | 0.950734888 | 0.930907501 |
| 4960 | 0.851619593 | 0.331978786 | 0.668021214 | 0.951727694 | 0.93216978 |
| 4970 | 0.853602902 | 0.332077489 | 0.667922511 | 0.952717361 | 0.933428363 |
| 4980 | 0.855582276 | 0.33217579 | 0.66782421 | 0.953703904 | 0.934683264 |
| 4990 | 0.857557717 | 0.332273692 | 0.667726308 | 0.954687334 | 0.935934495 |
| 5000 | 0.814837775 | 0.333687912 | 0.666312088 | 0.96075482 | 0.940944138 |
| 5010 | 0.816819462 | 0.333787855 | 0.666212145 | 0.961739327 | 0.942208202 |
| 5020 | 0.818797749 | 0.333887394 | 0.666112606 | 0.962721075 | 0.943469053 |
| 5030 | 0.82077263 | 0.333986529 | 0.666013471 | 0.963700073 | 0.944726699 |
| 5040 | 0.822744101 | 0.334085264 | 0.665914736 | 0.964676331 | 0.945981148 |
| 5050 | 0.824712155 | 0.334183601 | 0.665816399 | 0.965649856 | 0.947232408 |
| 5060 | 0.826676788 | 0.334281542 | 0.665718458 | 0.966620659 | 0.948480486 |
| 5070 | 0.828637994 | 0.334379089 | 0.665620911 | 0.967588747 | 0.949725391 |
| 5080 | 0.830595768 | 0.334476245 | 0.665523755 | 0.96855413 | 0.95096713 |
| 5090 | 0.832550107 | 0.33457301 | 0.66542699 | 0.969516816 | 0.95220571 |
| 5100 | 0.834501005 | 0.334669389 | 0.665330611 | 0.970476814 | 0.953441141 |
| 5110 | 0.836448458 | 0.334765383 | 0.665234617 | 0.971434133 | 0.95467343 |
| 5120 | 0.838392463 | 0.334860993 | 0.665139007 | 0.972388781 | 0.955902584 |
| 5130 | 0.840333014 | 0.334956223 | 0.665043777 | 0.973340767 | 0.957128611 |
| 5140 | 0.842270109 | 0.335051074 | 0.664948926 | 0.974290101 | 0.95835152 |
| 5150 | 0.844203743 | 0.335145548 | 0.664854452 | 0.975236789 | 0.959571318 |
| 5160 | 0.846133913 | 0.335239647 | 0.664760353 | 0.976180842 | 0.960788013 |
| 5170 | 0.848060616 | 0.335333375 | 0.664666625 | 0.977122267 | 0.962001613 |
| 5180 | 0.849983848 | 0.335426731 | 0.664573269 | 0.978061073 | 0.963212126 |
| 5190 | 0.851903606 | 0.33551972 | 0.66448028 | 0.978997268 | 0.964419559 |
| 5200 | 0.853819888 | 0.335612342 | 0.664387658 | 0.979930862 | 0.965623921 |
| 5210 | 0.855732691 | 0.335704599 | 0.664295401 | 0.980861863 | 0.966825219 |
| 5220 | 0.857642012 | 0.335796494 | 0.664203506 | 0.981790278 | 0.968023462 |
| 5230 | 0.859547848 | 0.335888029 | 0.664111971 | 0.982716117 | 0.969218656 |
| 5240 | 0.861450197 | 0.335979206 | 0.664020794 | 0.983639388 | 0.97041081 |
| 5250 | 0.863349057 | 0.336070026 | 0.663929974 | 0.984560099 | 0.971599932 |
| 5260 | 0.865244426 | 0.336160491 | 0.663839509 | 0.985478259 | 0.972786029 |
| 5270 | 0.867136301 | 0.336250604 | 0.663749396 | 0.986393876 | 0.97396911 |
| 5280 | 0.869024682 | 0.336340366 | 0.663659634 | 0.987306958 | 0.975149181 |
| 5290 | 0.870909566 | 0.33642978 | 0.66357022 | 0.988217513 | 0.976326252 |
| 5300 | 0.872790952 | 0.336518846 | 0.663481154 | 0.98912555 | 0.977500329 |
| 5310 | 0.874668837 | 0.336607568 | 0.663392432 | 0.990031077 | 0.978671421 |
| 5320 | 0.876543222 | 0.336695947 | 0.663304053 | 0.990934102 | 0.979839535 |
| 5330 | 0.878414105 | 0.336783984 | 0.663216016 | 0.991834634 | 0.98100468 |
| 5340 | 0.880281484 | 0.336871682 | 0.663128318 | 0.99273268 | 0.982166862 |
| 5350 | 0.882145359 | 0.336959042 | 0.663040958 | 0.993628248 | 0.98332609 |
| 5360 | 0.884005728 | 0.337046066 | 0.662953934 | 0.994521347 | 0.984482372 |
| 5370 | 0.885862592 | 0.337132757 | 0.662867243 | 0.995411985 | 0.985635714 |
| 5380 | 0.887715949 | 0.337219115 | 0.662780885 | 0.99630017 | 0.986786126 |
| 5390 | 0.889565799 | 0.337305143 | 0.662694857 | 0.997185909 | 0.987933614 |
| 5400 | 0.891412142 | 0.337390843 | 0.662609157 | 0.998069212 | 0.989078187 |
| 5410 | 0.893254977 | 0.337476215 | 0.662523785 | 0.998950084 | 0.990219852 |
| 5420 | 0.895094304 | 0.337561262 | 0.662438738 | 0.999828536 | 0.991358617 |
| 5430 | 0.896930123 | 0.337645986 | 0.662354014 | 1.000704574 | 0.99249449 |
| 5440 | 0.898762434 | 0.337730388 | 0.662269612 | 1.001578207 | 0.993627478 |
| 5450 | 0.900591237 | 0.337814471 | 0.662185529 | 1.002449441 | 0.994757588 |
| 5460 | 0.902416533 | 0.337898235 | 0.662101765 | 1.003318286 | 0.99588483 |
| 5470 | 0.904238321 | 0.337981682 | 0.662018318 | 1.004184749 | 0.99700921 |
| 5480 | 0.906056602 | 0.338064815 | 0.661935185 | 1.005048838 | 0.998130736 |
| 5490 | 0.907871377 | 0.338147634 | 0.661852366 | 1.00591056 | 0.999249415 |
| 5500 | 0.909682646 | 0.338230141 | 0.661769859 | 1.006769923 | 1.000365256 |
| 5510 | 0.911490411 | 0.338312339 | 0.661687661 | 1.007626935 | 1.001478266 |
| 5520 | 0.913294671 | 0.338394228 | 0.661605772 | 1.008481604 | 1.002588452 |
| 5530 | 0.915095428 | 0.338475811 | 0.661524189 | 1.009333937 | 1.003695822 |
| 5540 | 0.916892682 | 0.338557089 | 0.661442911 | 1.010183942 | 1.004800384 |
| 5550 | 0.918686436 | 0.338638063 | 0.661361937 | 1.011031626 | 1.005902146 |
| 5560 | 0.920476689 | 0.338718735 | 0.661281265 | 1.011876997 | 1.007001114 |
| 5570 | 0.922263443 | 0.338799106 | 0.661200894 | 1.012720063 | 1.008097297 |
| 5580 | 0.9240467 | 0.338879179 | 0.661120821 | 1.013560831 | 1.009190702 |
| 5590 | 0.925826461 | 0.338958955 | 0.661041045 | 1.014399308 | 1.010281336 |
| 5600 | 0.927602728 | 0.339038435 | 0.660961565 | 1.015235503 | 1.011369208 |
| 5610 | 0.929375502 | 0.339117621 | 0.660882379 | 1.016069422 | 1.012454324 |
| 5620 | 0.931144784 | 0.339196514 | 0.660803486 | 1.016901073 | 1.013536692 |
| 5630 | 0.932910577 | 0.339275117 | 0.660724883 | 1.017730463 | 1.014616319 |
| 5640 | 0.934672882 | 0.339353429 | 0.660646571 | 1.0185576 | 1.015693214 |
| 5650 | 0.936431702 | 0.339431454 | 0.660568546 | 1.019382491 | 1.016767383 |
| 5660 | 0.938187037 | 0.339509191 | 0.660490809 | 1.020205143 | 1.017838834 |
| 5670 | 0.939938891 | 0.339586644 | 0.660413356 | 1.021025564 | 1.018907575 |
| 5680 | 0.941687266 | 0.339663813 | 0.660336187 | 1.021843761 | 1.019973612 |
| 5690 | 0.943432163 | 0.3397407 | 0.6602593 | 1.022659741 | 1.021036954 |
| 5700 | 0.945173585 | 0.339817306 | 0.660182694 | 1.023473511 | 1.022097607 |
| 5710 | 0.946911534 | 0.339893632 | 0.660106368 | 1.024285079 | 1.023155579 |
| 5720 | 0.948646013 | 0.339969681 | 0.660030319 | 1.025094452 | 1.024210877 |
| 5730 | 0.950377024 | 0.340045453 | 0.659954547 | 1.025901636 | 1.025263509 |
| 5740 | 0.952104569 | 0.34012095 | 0.65987905 | 1.02670664 | 1.026313483 |
| 5750 | 0.953828652 | 0.340196173 | 0.659803827 | 1.027509469 | 1.027360804 |
| 5760 | 0.955549275 | 0.340271124 | 0.659728876 | 1.028310132 | 1.028405481 |
| 5770 | 0.957266441 | 0.340345804 | 0.659654196 | 1.029108635 | 1.029447522 |
| 5780 | 0.958980152 | 0.340420215 | 0.659579785 | 1.029904985 | 1.030486932 |
| 5790 | 0.960690412 | 0.340494357 | 0.659505643 | 1.030699189 | 1.03152372 |
| 5800 | 0.962397223 | 0.340568232 | 0.659431768 | 1.031491255 | 1.032557893 |
| 5810 | 0.964100588 | 0.340641842 | 0.659358158 | 1.032281188 | 1.033589458 |
| 5820 | 0.965800511 | 0.340715188 | 0.659284812 | 1.033068997 | 1.034618422 |
| 5830 | 0.967496994 | 0.34078827 | 0.65921173 | 1.033854688 | 1.035644792 |
| 5840 | 0.969190041 | 0.340861092 | 0.659138908 | 1.034638267 | 1.036668576 |
| 5850 | 0.970879655 | 0.340933653 | 0.659066347 | 1.035419742 | 1.037689781 |
| 5860 | 0.972565839 | 0.341005955 | 0.658994045 | 1.03619912 | 1.038708414 |
| 5870 | 0.974248597 | 0.341077999 | 0.658922001 | 1.036976407 | 1.039724482 |
| 5880 | 0.975927931 | 0.341149787 | 0.658850213 | 1.03775161 | 1.040737993 |
| 5890 | 0.977603846 | 0.34122132 | 0.65877868 | 1.038524735 | 1.041748953 |
| 5900 | 0.979276345 | 0.341292599 | 0.658707401 | 1.039295791 | 1.042757369 |
| 5910 | 0.980945431 | 0.341363626 | 0.658636374 | 1.040064783 | 1.043763249 |
| 5920 | 0.982611109 | 0.341434401 | 0.658565599 | 1.040831717 | 1.0447666 |
| 5930 | 0.984273381 | 0.341504926 | 0.658495074 | 1.041596601 | 1.045767428 |
| 5940 | 0.985932251 | 0.341575203 | 0.658424797 | 1.042359442 | 1.046765741 |
| 5950 | 0.987587724 | 0.341645231 | 0.658354769 | 1.043120245 | 1.047761546 |
| 5960 | 0.989239802 | 0.341715014 | 0.658284986 | 1.043879018 | 1.04875485 |
| 5970 | 0.99088849 | 0.341784551 | 0.658215449 | 1.044635767 | 1.04974566 |
| 5980 | 0.992533792 | 0.341853844 | 0.658146156 | 1.045390498 | 1.050733982 |
| 5990 | 0.994175712 | 0.341922894 | 0.658077106 | 1.046143219 | 1.051719824 |
| 6000 | 0.995814253 | 0.341991703 | 0.658008297 | 1.046893935 | 1.052703193 |
| 6010 | 0.99744942 | 0.342060272 | 0.657939728 | 1.047642653 | 1.053684096 |
| 6020 | 0.999081217 | 0.342128601 | 0.657871399 | 1.048389379 | 1.054662539 |
| 6030 | 1.000709647 | 0.342196692 | 0.657803308 | 1.049134121 | 1.05563853 |
| 6040 | 1.002334715 | 0.342264546 | 0.657735454 | 1.049876884 | 1.056612075 |
| 6050 | 1.003956426 | 0.342332165 | 0.657667835 | 1.050617674 | 1.057583181 |
| 6060 | 1.005574783 | 0.342399548 | 0.657600452 | 1.051356499 | 1.058551856 |
| 6070 | 1.00718979 | 0.342466699 | 0.657533301 | 1.052093364 | 1.059518105 |
| 6080 | 1.008801453 | 0.342533617 | 0.657466383 | 1.052828276 | 1.060481936 |
| 6090 | 1.010409775 | 0.342600303 | 0.657399697 | 1.053561241 | 1.061443356 |
| 6100 | 1.01201476 | 0.34266676 | 0.65733324 | 1.054292265 | 1.062402371 |
| 6110 | 1.013616414 | 0.342732988 | 0.657267012 | 1.055021354 | 1.063358988 |
| 6120 | 1.015214741 | 0.342798987 | 0.657201013 | 1.055748516 | 1.064313214 |
| 6130 | 1.016809744 | 0.34286476 | 0.65713524 | 1.056473755 | 1.065265056 |
| 6140 | 1.018401429 | 0.342930307 | 0.657069693 | 1.057197079 | 1.06621452 |
| 6150 | 1.0199898 | 0.34299563 | 0.65700437 | 1.057918493 | 1.067161613 |
| 6160 | 1.021574863 | 0.343060729 | 0.656939271 | 1.058638003 | 1.068106342 |
| 6170 | 1.02315662 | 0.343125605 | 0.656874395 | 1.059355616 | 1.069048714 |
| 6180 | 1.024735078 | 0.34319026 | 0.65680974 | 1.060071337 | 1.069988734 |
| 6190 | 1.026310241 | 0.343254694 | 0.656745306 | 1.060785174 | 1.070926411 |
| 6200 | 1.027882113 | 0.34331891 | 0.65668109 | 1.061497131 | 1.07186175 |
| 6210 | 1.029450699 | 0.343382906 | 0.656617094 | 1.062207214 | 1.072794757 |
| 6220 | 1.031016005 | 0.343446686 | 0.656553314 | 1.062915431 | 1.073725441 |
| 6230 | 1.032578034 | 0.343510249 | 0.656489751 | 1.063621787 | 1.074653806 |
| 6240 | 1.034136793 | 0.343573597 | 0.656426403 | 1.064326287 | 1.075579861 |
| 6250 | 1.035692285 | 0.343636731 | 0.656363269 | 1.065028938 | 1.076503611 |
| 6260 | 1.037244515 | 0.343699652 | 0.656300348 | 1.065729746 | 1.077425062 |
| 6270 | 1.038793489 | 0.34376236 | 0.65623764 | 1.066428716 | 1.078344222 |
| 6280 | 1.040339212 | 0.343824858 | 0.656175142 | 1.067125854 | 1.079261097 |
| 6290 | 1.041881688 | 0.343887145 | 0.656112855 | 1.067821167 | 1.080175693 |
| 6300 | 1.043420923 | 0.343949222 | 0.656050778 | 1.06851466 | 1.081088017 |
| 6310 | 1.044956921 | 0.344011092 | 0.655988908 | 1.069206339 | 1.081998075 |
| 6320 | 1.046489687 | 0.344072754 | 0.655927246 | 1.06989621 | 1.082905874 |
| 6330 | 1.048019228 | 0.34413421 | 0.65586579 | 1.070584278 | 1.08381142 |
| 6340 | 1.049545547 | 0.344195461 | 0.655804539 | 1.07127055 | 1.08471472 |
| 6350 | 1.05106865 | 0.344256507 | 0.655743493 | 1.07195503 | 1.085615779 |
| 6360 | 1.052588543 | 0.34431735 | 0.65568265 | 1.072637725 | 1.086514605 |
| 6370 | 1.054105229 | 0.34437799 | 0.65562201 | 1.073318641 | 1.087411204 |
| 6380 | 1.055618715 | 0.344438428 | 0.655561572 | 1.073997783 | 1.088305581 |
| 6390 | 1.057129006 | 0.344498666 | 0.655501334 | 1.074675156 | 1.089197744 |
| 6400 | 1.058636107 | 0.344558705 | 0.655441295 | 1.075350767 | 1.090087698 |
| 6410 | 1.060140023 | 0.344618544 | 0.655381456 | 1.076024621 | 1.09097545 |
| 6420 | 1.06164076 | 0.344678186 | 0.655321814 | 1.076696723 | 1.091861006 |
| 6430 | 1.063138323 | 0.34473763 | 0.65526237 | 1.07736708 | 1.092744373 |
| 6440 | 1.064632716 | 0.344796879 | 0.655203121 | 1.078035697 | 1.093625557 |
| 6450 | 1.066123947 | 0.344855932 | 0.655144068 | 1.078702578 | 1.094504563 |
| 6460 | 1.067612019 | 0.344914791 | 0.655085209 | 1.079367731 | 1.095381398 |
| 6470 | 1.069096939 | 0.344973457 | 0.655026543 | 1.080031159 | 1.096256069 |
| 6480 | 1.070578711 | 0.34503193 | 0.65496807 | 1.08069287 | 1.097128581 |
| 6490 | 1.072057342 | 0.345090212 | 0.654909788 | 1.081352867 | 1.09799894 |
| 6500 | 1.073532836 | 0.345148302 | 0.654851698 | 1.082011158 | 1.098867154 |
| 6510 | 1.075005199 | 0.345206203 | 0.654793797 | 1.082667746 | 1.099733227 |
| 6520 | 1.076474436 | 0.345263915 | 0.654736085 | 1.083322638 | 1.100597166 |
| 6530 | 1.077940554 | 0.345321439 | 0.654678561 | 1.083975839 | 1.101458977 |
| 6540 | 1.079403557 | 0.345378775 | 0.654621225 | 1.084627353 | 1.102318666 |
| 6550 | 1.080863451 | 0.345435925 | 0.654564075 | 1.085277188 | 1.10317624 |
| 6560 | 1.082320242 | 0.345492889 | 0.654507111 | 1.085925347 | 1.104031704 |
| 6570 | 1.083773935 | 0.345549668 | 0.654450332 | 1.086571836 | 1.104885064 |
| 6580 | 1.085224535 | 0.345606264 | 0.654393736 | 1.087216661 | 1.105736326 |
| 6590 | 1.086672049 | 0.345662676 | 0.654337324 | 1.087859827 | 1.106585496 |
| 6600 | 1.088116482 | 0.345718906 | 0.654281094 | 1.088501339 | 1.107432581 |
| 6610 | 1.089557839 | 0.345774955 | 0.654225045 | 1.089141201 | 1.108277586 |
| 6620 | 1.090996126 | 0.345830822 | 0.654169178 | 1.089779421 | 1.109120517 |
| 6630 | 1.092431349 | 0.34588651 | 0.65411349 | 1.090416001 | 1.10996138 |
| 6640 | 1.093863513 | 0.345942019 | 0.654057981 | 1.091050949 | 1.110800182 |
| 6650 | 1.095292624 | 0.345997349 | 0.654002651 | 1.091684268 | 1.111636927 |
| 6660 | 1.096718688 | 0.346052502 | 0.653947498 | 1.092315965 | 1.112471621 |
| 6670 | 1.09814171 | 0.346107479 | 0.653892521 | 1.092946044 | 1.113304272 |
| 6680 | 1.099561696 | 0.346162279 | 0.653837721 | 1.09357451 | 1.114134884 |
| 6690 | 1.100978651 | 0.346216904 | 0.653783096 | 1.094201368 | 1.114963463 |
| 6700 | 1.102392582 | 0.346271355 | 0.653728645 | 1.094826624 | 1.115790015 |
| 6710 | 1.103803495 | 0.346325632 | 0.653674368 | 1.095450282 | 1.116614546 |
| 6720 | 1.105211394 | 0.346379737 | 0.653620263 | 1.096072348 | 1.117437062 |
| 6730 | 1.106616285 | 0.346433669 | 0.653566331 | 1.096692826 | 1.118257569 |
| 6740 | 1.108018175 | 0.34648743 | 0.65351257 | 1.097311722 | 1.119076071 |
| 6750 | 1.109417069 | 0.346541021 | 0.653458979 | 1.09792904 | 1.119892576 |
| 6760 | 1.110812972 | 0.346594441 | 0.653405559 | 1.098544786 | 1.120707088 |
| 6770 | 1.112205891 | 0.346647693 | 0.653352307 | 1.099158964 | 1.121519614 |
| 6780 | 1.113595832 | 0.346700776 | 0.653299224 | 1.09977158 | 1.122330158 |
| 6790 | 1.1149828 | 0.346753692 | 0.653246308 | 1.100382637 | 1.123138728 |
| 6800 | 1.1163668 | 0.346806441 | 0.653193559 | 1.100992142 | 1.123945328 |
| 6810 | 1.117747839 | 0.346859024 | 0.653140976 | 1.101600099 | 1.124749963 |
| 6820 | 1.119125923 | 0.346911441 | 0.653088559 | 1.102206512 | 1.125552641 |
| 6830 | 1.120501057 | 0.346963693 | 0.653036307 | 1.102811387 | 1.126353366 |
| 6840 | 1.121873247 | 0.347015782 | 0.652984218 | 1.103414729 | 1.127152143 |
| 6850 | 1.123242499 | 0.347067707 | 0.652932293 | 1.104016542 | 1.127948979 |
| 6860 | 1.124608818 | 0.34711947 | 0.65288053 | 1.104616831 | 1.128743879 |
| 6870 | 1.125972212 | 0.347171071 | 0.652828929 | 1.105215601 | 1.129536849 |
| 6880 | 1.127332684 | 0.347222511 | 0.652777489 | 1.105812856 | 1.130327894 |
| 6890 | 1.128690242 | 0.34727379 | 0.65272621 | 1.106408602 | 1.131117019 |
| 6900 | 1.130044891 | 0.34732491 | 0.65267509 | 1.107002842 | 1.13190423 |
| 6910 | 1.131396637 | 0.34737587 | 0.65262413 | 1.107595583 | 1.132689533 |
| 6920 | 1.132745486 | 0.347426672 | 0.652573328 | 1.108186828 | 1.133472933 |
| 6930 | 1.134091443 | 0.347477316 | 0.652522684 | 1.108776582 | 1.134254436 |
| 6940 | 1.135434515 | 0.347527804 | 0.652472196 | 1.109364849 | 1.135034046 |
| 6950 | 1.136774707 | 0.347578135 | 0.652421865 | 1.109951635 | 1.13581177 |
| 6960 | 1.138112025 | 0.34762831 | 0.65237169 | 1.110536944 | 1.136587612 |
| 6970 | 1.139446475 | 0.34767833 | 0.65232167 | 1.111120781 | 1.137361579 |
| 6980 | 1.140778063 | 0.347728196 | 0.652271804 | 1.11170315 | 1.138133675 |
| 6990 | 1.142106796 | 0.347777908 | 0.652222092 | 1.112284055 | 1.138903906 |
| 7000 | 1.143432677 | 0.347827467 | 0.652172533 | 1.112863502 | 1.139672277 |
| 7010 | 1.144766237 | 0.347877266 | 0.652122734 | 1.113446091 | 1.140444889 |
| 7020 | 1.146092396 | 0.347926743 | 0.652073257 | 1.114025234 | 1.141213007 |
| 7030 | 1.147415683 | 0.347976067 | 0.652023933 | 1.114602914 | 1.141979256 |
| 7040 | 1.148736107 | 0.348025238 | 0.651974762 | 1.115179135 | 1.142743644 |
| 7050 | 1.150053676 | 0.348074258 | 0.651925742 | 1.115753903 | 1.143506177 |
| 7060 | 1.151368395 | 0.348123127 | 0.651876873 | 1.116327223 | 1.14426686 |
| 7070 | 1.152680273 | 0.348171846 | 0.651828154 | 1.1168991 | 1.1450257 |
| 7080 | 1.153989317 | 0.348220415 | 0.651779585 | 1.117469539 | 1.145782703 |
| 7090 | 1.155295533 | 0.348268836 | 0.651731164 | 1.118038544 | 1.146537875 |
| 7100 | 1.15659893 | 0.348317108 | 0.651682892 | 1.118606121 | 1.147291221 |
| 7110 | 1.157899514 | 0.348365233 | 0.651634767 | 1.119172276 | 1.148042748 |
| 7120 | 1.159197292 | 0.348413211 | 0.651586789 | 1.119737012 | 1.148792462 |
| 7130 | 1.160492272 | 0.348461043 | 0.651538957 | 1.120300334 | 1.149540368 |
| 7140 | 1.161784461 | 0.348508729 | 0.651491271 | 1.120862249 | 1.150286473 |
| 7150 | 1.163073865 | 0.348556271 | 0.651443729 | 1.12142276 | 1.151030781 |
| 7160 | 1.164360493 | 0.348603668 | 0.651396332 | 1.121981872 | 1.1517733 |
| 7170 | 1.16564435 | 0.348650922 | 0.651349078 | 1.12253959 | 1.152514035 |
| 7180 | 1.166925445 | 0.348698033 | 0.651301967 | 1.12309592 | 1.153252991 |
| 7190 | 1.168203784 | 0.348745002 | 0.651254998 | 1.123650865 | 1.153990175 |
| 7200 | 1.169479374 | 0.348791829 | 0.651208171 | 1.124204431 | 1.154725592 |
| 7210 | 1.170752223 | 0.348838515 | 0.651161485 | 1.124756623 | 1.155459248 |
| 7220 | 1.172022337 | 0.34888506 | 0.65111494 | 1.125307444 | 1.156191148 |
| 7230 | 1.173289723 | 0.348931466 | 0.651068534 | 1.1258569 | 1.156921299 |
| 7240 | 1.174554389 | 0.348977732 | 0.651022268 | 1.126404996 | 1.157649706 |
| 7250 | 1.175816341 | 0.34902386 | 0.65097614 | 1.126951736 | 1.158376374 |
| 7260 | 1.177075586 | 0.349069851 | 0.650930149 | 1.127497124 | 1.159101309 |
| 7270 | 1.178332131 | 0.349115703 | 0.650884297 | 1.128041166 | 1.159824517 |
| 7280 | 1.179585984 | 0.34916142 | 0.65083858 | 1.128583866 | 1.160546003 |
| 7290 | 1.180837151 | 0.349206999 | 0.650793001 | 1.129125229 | 1.161265773 |
| 7300 | 1.182085638 | 0.349252444 | 0.650747556 | 1.129665259 | 1.161983832 |
| 7310 | 1.183331454 | 0.349297753 | 0.650702247 | 1.13020396 | 1.162700186 |
| 7320 | 1.184574604 | 0.349342929 | 0.650657071 | 1.130741338 | 1.16341484 |
| 7330 | 1.185815096 | 0.34938797 | 0.65061203 | 1.131277396 | 1.164127799 |
| 7340 | 1.187052936 | 0.349432878 | 0.650567122 | 1.13181214 | 1.16483907 |
| 7350 | 1.188288131 | 0.349477654 | 0.650522346 | 1.132345573 | 1.165548658 |
| 7360 | 1.189520689 | 0.349522297 | 0.650477703 | 1.132877701 | 1.166256567 |
| 7370 | 1.190750615 | 0.34956681 | 0.65043319 | 1.133408527 | 1.166962803 |
| 7380 | 1.191977917 | 0.349611191 | 0.650388809 | 1.133938056 | 1.167667372 |
| 7390 | 1.193202601 | 0.349655442 | 0.650344558 | 1.134466292 | 1.168370279 |
| 7400 | 1.194424675 | 0.349699563 | 0.650300437 | 1.13499324 | 1.169071529 |
| 7410 | 1.195644144 | 0.349743555 | 0.650256445 | 1.135518904 | 1.169771128 |
| 7420 | 1.196861016 | 0.349787418 | 0.650212582 | 1.136043288 | 1.17046908 |
| 7430 | 1.198075298 | 0.349831153 | 0.650168847 | 1.136566397 | 1.171165391 |
| 7440 | 1.199286995 | 0.349874761 | 0.650125239 | 1.137088235 | 1.171860066 |
| 7450 | 1.200496115 | 0.349918242 | 0.650081758 | 1.137608806 | 1.172553111 |
| 7460 | 1.201702664 | 0.349961596 | 0.650038404 | 1.138128115 | 1.17324453 |
| 7470 | 1.202906649 | 0.350004824 | 0.649995176 | 1.138646165 | 1.173934329 |
| 7480 | 1.204108077 | 0.350047927 | 0.649952073 | 1.139162962 | 1.174622512 |
| 7490 | 1.205306954 | 0.350090905 | 0.649909095 | 1.139678508 | 1.175309085 |
| 7500 | 1.206503287 | 0.350133759 | 0.649866241 | 1.140192809 | 1.175994054 |
| 7510 | 1.207697082 | 0.350176489 | 0.649823511 | 1.140705868 | 1.176677422 |
| 7520 | 1.208888346 | 0.350219096 | 0.649780904 | 1.14121769 | 1.177359196 |
| 7530 | 1.210077086 | 0.35026158 | 0.64973842 | 1.141728279 | 1.178039379 |
| 7540 | 1.211263307 | 0.350303942 | 0.649696058 | 1.142237638 | 1.178717978 |
| 7550 | 1.212447017 | 0.350346182 | 0.649653818 | 1.142745773 | 1.179394997 |
| 7560 | 1.213628222 | 0.3503883 | 0.6496117 | 1.143252686 | 1.180070441 |
| 7570 | 1.214806929 | 0.350430299 | 0.649569701 | 1.143758383 | 1.180744315 |
| 7580 | 1.215983143 | 0.350472176 | 0.649527824 | 1.144262867 | 1.181416623 |
| 7590 | 1.217156872 | 0.350513935 | 0.649486065 | 1.144766141 | 1.182087372 |
| 7600 | 1.218328122 | 0.350555574 | 0.649444426 | 1.145268211 | 1.182756566 |
| 7610 | 1.219496899 | 0.350597094 | 0.649402906 | 1.14576908 | 1.183424209 |
| 7620 | 1.22066321 | 0.350638496 | 0.649361504 | 1.146268753 | 1.184090306 |
| 7630 | 1.221827061 | 0.350679781 | 0.649320219 | 1.146767232 | 1.184754863 |
| 7640 | 1.222988458 | 0.350720948 | 0.649279052 | 1.147264522 | 1.185417885 |
| 7650 | 1.224147408 | 0.350761999 | 0.649238001 | 1.147760627 | 1.186079375 |
| 7660 | 1.225303917 | 0.350802933 | 0.649197067 | 1.148255551 | 1.186739338 |
| 7670 | 1.226457992 | 0.350843752 | 0.649156248 | 1.148749297 | 1.187397781 |
| 7680 | 1.227609638 | 0.350884455 | 0.649115545 | 1.14924187 | 1.188054706 |
| 7690 | 1.228758863 | 0.350925043 | 0.649074957 | 1.149733274 | 1.18871012 |
| 7700 | 1.229905672 | 0.350965517 | 0.649034483 | 1.150223512 | 1.189364026 |
| 7710 | 1.231050072 | 0.351005878 | 0.648994122 | 1.150712587 | 1.190016429 |
| 7720 | 1.232192068 | 0.351046125 | 0.648953875 | 1.151200505 | 1.190667334 |
| 7730 | 1.233331668 | 0.351086259 | 0.648913741 | 1.151687268 | 1.191316747 |
| 7740 | 1.234468877 | 0.35112628 | 0.64887372 | 1.152172881 | 1.19196467 |
| 7750 | 1.235603702 | 0.35116619 | 0.64883381 | 1.152657347 | 1.192611109 |
| 7760 | 1.236736148 | 0.351205987 | 0.648794013 | 1.15314067 | 1.193256069 |
| 7770 | 1.237866222 | 0.351245674 | 0.648754326 | 1.153622853 | 1.193899554 |
| 7780 | 1.238993931 | 0.351285251 | 0.648714749 | 1.154103901 | 1.194541568 |
| 7790 | 1.240119279 | 0.351324717 | 0.648675283 | 1.154583817 | 1.195182117 |
| 7800 | 1.241242274 | 0.351364073 | 0.648635927 | 1.155062605 | 1.195821205 |
| 7810 | 1.242362921 | 0.35140332 | 0.64859668 | 1.155540268 | 1.196458835 |
| 7820 | 1.243481227 | 0.351442458 | 0.648557542 | 1.15601681 | 1.197095014 |
| 7830 | 1.244597198 | 0.351481488 | 0.648518512 | 1.156492235 | 1.197729744 |
| 7840 | 1.245710839 | 0.351520409 | 0.648479591 | 1.156966546 | 1.198363032 |
| 7850 | 1.246822157 | 0.351559224 | 0.648440776 | 1.157439746 | 1.19899488 |
| 7860 | 1.247931158 | 0.351597931 | 0.648402069 | 1.157911841 | 1.199625294 |
| 7870 | 1.249037847 | 0.351636531 | 0.648363469 | 1.158382832 | 1.200254278 |
| 7880 | 1.250142232 | 0.351675025 | 0.648324975 | 1.158852724 | 1.200881836 |
| 7890 | 1.251244317 | 0.351713414 | 0.648286586 | 1.15932152 | 1.201507973 |
| 7900 | 1.252344109 | 0.351751697 | 0.648248303 | 1.159789224 | 1.202132693 |
| 7910 | 1.253441613 | 0.351789875 | 0.648210125 | 1.160255839 | 1.202756 |
| 7920 | 1.254536836 | 0.351827948 | 0.648172052 | 1.160721369 | 1.203377899 |
| 7930 | 1.255629784 | 0.351865917 | 0.648134083 | 1.161185817 | 1.203998394 |
| 7940 | 1.256720462 | 0.351903783 | 0.648096217 | 1.161649187 | 1.20461749 |
| 7950 | 1.257808877 | 0.351941545 | 0.648058455 | 1.162111481 | 1.20523519 |
| 7960 | 1.258895034 | 0.351979204 | 0.648020796 | 1.162572705 | 1.205851498 |
| 7970 | 1.259978939 | 0.352016761 | 0.647983239 | 1.16303286 | 1.20646642 |
| 7980 | 1.261060598 | 0.352054216 | 0.647945784 | 1.163491951 | 1.20707996 |
| 7990 | 1.262140017 | 0.352091569 | 0.647908431 | 1.16394998 | 1.207692121 |
| 8000 | 1.263217201 | 0.352128821 | 0.647871179 | 1.164406952 | 1.208302907 |

TABLE 3
Numerical values for photoreceptor class-to-photopic luminance activation ratios with correlated color
temperatures (CCT) and the component emissions for specified with reference to a 12-bit scale (S1-S9)

| CCT | Photoreceptor excitations | | | | | | Component emissions | | | | | | | | |
|---|---|---|---|---|---|---|---|---|---|---|---|---|---|---|---|
| (K) | S | M | L | R | I | CRI | S1 | S2 | S3 | S4 | S5 | S6 | S7 | S8 | S9 |
| 3000 | 0.3907 | 0.3002 | 0.6998 | 0.6762 | 0.5950 | 98 | 1725 | 344 | 187 | 749 | 865 | 1184 | 3116 | 2045 | 0 |
| 4000 | 0.6427 | 0.3202 | 0.6798 | 0.8400 | 0.7920 | 98 | 1724 | 1250 | 484 | 1119 | 765 | 1902 | 1995 | 1332 | 2 |
| 5000 | 0.8148 | 0.3337 | 0.6663 | 0.9608 | 0.9409 | 98 | 2141 | 1688 | 560 | 1460 | 425 | 1793 | 404 | 1 | 236 |
| 6000 | 0.9958 | 0.342 | 0.6580 | 1.0469 | 1.0527 | 98 | 2151 | 2263 | 920 | 1283 | 1116 | 1311 | 175 | 29 | 25 |
| 7000 | 1.1434 | 0.3478 | 0.6522 | 1.1129 | 1.1397 | 98 | 2093 | 2656 | 1374 | 918 | 1953 | 935 | 7 | 4 | 7 |

### REFERENCES

### Rod Contributions to Color Perception:

Cao D, Pokorny J, Smith V C, Zele A J. (2008). Rod contribution to color perception: Linear with rod contrast. Vision Research. 48. 2586-2592.
Cao D, Zele A J, Pokorny J. (2008). Chromatic discrimination in the presence of incremental and decremental rod pedestals. Visual Neuroscience. 25. 399-404.

### Spectral Sensitivity:

Feigl B, Zele A J. (2014). Melanopsin expressing intrinsically photosensitive Retinal Ganglion Cells in retinal disease. Optometry and Vision Science. 91. 894-903.
Adhikari P, Zele A J, Feigl B. (2015). The post-illumination pupil response (PIPR). Investigative Ophthalmology and Visual Science. 56. 3838-3849.
Adhikari P, Feigl B, Zele A J. (2016). Rhodopsin and melanopsin contributions to the early redilation phase of the Post-Illumination Pupil Response (PIPR). PLoS One. 11(8): e0161175. doi:10.1371/journal.pone.0161175.
Enezi, J., Revell, V., Brown, T., Wynne, J., Schlangen, L., & Lucas, R. (2011). A “melanopic” spectral efficiency function predicts the sensitivity of melanopsin photoreceptors to polychromatic lights. Journal of Biological Rhythms, 26(4), 314-323.
MacLeod, D. I. A., & Boynton, R. M. (1979). Chromaticity diagram showing cone excitation by stimuli of equal luminance. Journal of the Optical Society of America, 69, 1183-1185.
Smith, V. C., & Pokorny, J. (1975). Spectral sensitivity of the foveal cone photopigments between 400 and 500 nm. Vision Research, 15(2), 161-171.

### Melanopsin Contributions to Brightness Perception:

Zele A J, Adhikari P, Feigl B, Cao D. (2018). Cone and melanopsin contributions to human brightness estimation. Journal of the Optical Society of America A. 35(4). B19-B25.
Zele A J, Dey A, Adhikari P, Feigl B. (2020). Rhodopsin and melanopsin contributions to human brightness estimation. Journal of the Optical Society of America A. 37(4). A145-A153

### Melanopsin Contributions to Color Perception:

Zele A J, Feigl B, Adhikari P, Maynard M L, Cao D. (2018). Melanopsin photoreception contributes to human visual detection, temporal and color processing. Scientific Reports. 8.3842. DOI:10.1038/s41598-018-22197-w
Zele A J, Adhikari P, Cao D, Feigl B. (2019). Melanopsin driven enhancement of cone-mediated visual processing. Vision Research. 160. 72-81.

### Melanopsin Contributions to the Perception of White:

Cao D, Chang A, Gai S. (2018). Evidence for an impact of melanopsin activation on unique white perception. Journal of the Optical Society of America A. 35(4). B287-B291.
   Rod and Melanopsin Expressing ipRGC Contributions to Sleep and Circadian Photoentrainment:
Altimus C M, Guler A D, Alam N M, Arman A C, Prusky G T, Sampath A P, Hatter S. (2010). Rod photoreceptors drive circadian photoentrainment across a wide range of light intensities. Nature Neuroscience. 13(9). 1107-1113.
Dumpala S, Zele A J, Feigl B. (2019). Outer retinal structure and function deficits contribute to circadian disruption in patients with type 2 diabetes. Investigative Ophthalmology and Visual Science. 2019; 60:1870-1878.

1. A lighting device providing emitted light with an adjustable correlated color temperature (CCT), the lighting device comprising:
one or more light emitters with each respective light emitter configured to provide one or more spectral components of the emitted light, wherein the emitted light comprises a respective photoreceptor class-to-photopic luminance activation ratio for each ocular photoreceptor class corresponding to an intended CCT of the emitted light, the respective photoreceptor class-to-photopic luminance activation ratios comprising:
(a) a melanopsin (i)-to-photopic-luminance activation ratio within a defined range of a melanopsin-to-photopic-luminance activation ratio of a blackbody radiator with the intended CCT;
(b) a rhodopsin (R)-to-photopic-luminance activation ratio within a defined range of a rhodopsin-to-photopic-luminance activation ratio of a blackbody radiator with the intended CCT;
(c) a long-wavelength sensitive opsin (L)-to-photopic-luminance activation ratio within a defined range of a long-wavelength sensitive opsin-to-photopic-luminance activation ratio of a blackbody radiator with the intended CCT;
(d) a middle-wavelength sensitive opsin (M)-to-photopic-luminance activation ratio within a defined range of a middle-wavelength sensitive opsin-to-photopic-luminance activation ratio of a blackbody radiator with the intended CCT; and
(e) a short-wavelength sensitive opsin (S)-to-photopic-luminance activation ratio within a defined range of a short-wavelength sensitive opsin-to-photopic-luminance activation ratio of a blackbody radiator with the intended CCT; and
a light emitter controller, configured to independently control each of the one or more light emitters to provide the emitted light.

2. A method for providing emitted light with an adjustable correlated color temperature (CCT), the method comprising:
independently controlling one or more light emitters to provide emitted light wherein each respective light emitter is configured to provide one or more spectral components of the emitted light, wherein the emitted light comprises a respective photoreceptor class-to-photopic luminance activation ratio for each ocular photoreceptor class corresponding to an intended CCT of the emitted light, the respective photoreceptor class-to-photopic luminance activation ratios comprising:
(a) a melanopsin (i)-to-photopic-luminance activation ratio within a defined range of a melanopsin-to-photopic-luminance activation ratio of a blackbody radiator with the intended CCT;
(b) a rhodopsin (R)-to-photopic-luminance activation ratio within a defined range of a rhodopsin-to-photopic-luminance activation ratio of a blackbody radiator with the intended CCT;
(c) a long-wavelength sensitive opsin (L)-to-photopic-luminance activation ratio within a defined range of a long-wavelength sensitive opsin-to-photopic-luminance activation ratio of a blackbody radiator with the intended CCT;
(d) a middle-wavelength sensitive opsin (M)-to-photopic-luminance activation ratio within a defined range of a middle-wavelength sensitive opsin-to-photopic-luminance activation ratio of a blackbody radiator with the intended CCT; and
(e) a short-wavelength sensitive opsin (S)-to-photopic-luminance activation ratio within a defined range of a short-wavelength sensitive opsin-to-photopic-luminance activation ratio of a blackbody radiator with the intended CCT.

3. A system for providing emitted light with an adjustable correlated color temperature (CCT), the system comprising:
one or more light emitters wherein each respective light emitter is configured to provide one or more spectral components of the emitted light, wherein the emitted light comprises a respective photoreceptor class-to-photopic luminance activation ratio for each ocular photoreceptor class corresponding to an intended CCT of the emitted light, the respective photoreceptor class-to-photopic luminance activation ratios comprising:
(a) a melanopsin (i)-to-photopic-luminance activation ratio within a defined range of a melanopsin-to-photopic-luminance activation ratio of a blackbody radiator with the intended CCT;
(b) a rhodopsin (R)-to-photopic-luminance activation ratio within a defined range of a rhodopsin-to-photopic-luminance activation ratio of a blackbody radiator with the intended CCT;
(c) a long-wavelength sensitive opsin (L)-to-photopic-luminance activation ratio within a defined range of a long-wavelength sensitive opsin-to-photopic-luminance activation ratio of a blackbody radiator with the intended CCT;
(d) a middle-wavelength sensitive opsin (M)-to-photopic-luminance activation ratio within a defined range of a middle-wavelength sensitive opsin-to-photopic-luminance activation ratio of a blackbody radiator with the intended CCT; and
(e) a short-wavelength sensitive opsin (S)-to-photopic-luminance activation ratio within a defined range of a short-wavelength sensitive opsin-to-photopic-luminance activation ratio of a blackbody radiator with the intended CCT; and
a light emitter controller, configured to independently control each of the one or more light emitters to provide the emitted light.

4.-7. (canceled)

8. The lighting device of claim 1, wherein the one or more light emitters comprise five light emitters, wherein each of the five light emitters emit light comprising one spectral component.

9. The lighting device, of claim 1 wherein at least one of the one or more light emitters emit light comprising two or more spectral components.

10. The lighting device of claim 1, wherein the emitted light produces a photoreceptor class-to-photopic-luminance activation defined by a respective cubic polynomial which is a function of CCT for each respective ocular photoreceptor class: i; R; L; M; and S.

11. The lighting device of claim 1, wherein the respective photoreceptor class-to-photopic luminance activation ratios are defined as being unit-normalized to equal-luminance.

12. The lighting device of claim 1, wherein the respective photoreceptor class-to-photopic luminance activation ratios are defined using one or more sensitivity function.

13. The lighting device according to of claim 10, wherein each respective cubic polynomial for each ocular photoreceptor class i; R; L; M; and S comprises ax³+bx²+cx+d where x is a desired CCT divided by 1,000.

14. (canceled)

15. The lighting device of claim 1, wherein the defined range for each respective ocular photoreceptor class comprises an increase and/or decrease relative to a target activation ratio of each respective photoreceptor class: i; R; L; M; and S.

16. The lighting device of claim 1, wherein an intended perceived color is maintained while a melanopsin (i) stimulation and/or rhodopsin (R) stimulation may be varied to simulate an effect of the sun at a same or different CCT.

17. The lighting device according to claim 10 wherein the defined range is defined, with reference to the polynomial, by
ax³+bx²+cx+d±B wherein a value for B for each respective ocular photoreceptor class i; R; L; M; and S comprises 0.01 to 0.1; 0.02 to 0.08; or 0.025 to 0.075.

18. The lighting device according to claim 10 wherein the defined range is defined, with reference to the polynomial, by
ax³+bx²+(c±(B/F))x+d wherein a value for B for each respective ocular photoreceptor class comprises 0.01 to 0.1; 0.02 to 0.08; or 0.025 to 0.075, wherein F is between 3.0 and 10.0.

19. (canceled)

20. (canceled)

21. The lighting device according to claim 17 or claim 18 wherein the defined range applies for a CCT greater than or equal to 3,000K.

22. The lighting device according to claim 17 or claim 18 wherein the value for B for
R=0.05; for L=0.025; for M=0.025; for S=0.075; and for ipRGC=0.05.

23.-27. (canceled)

28. The lighting device of claim 10, wherein coefficient values for each respective ocular photoreceptor class are as shown in Table 1.

29.-31. (canceled)

32. The lighting device of claim 1, wherein respective ocular photoreceptor activation ratio is independent of an illumination level of the emitted light.

33.-36. (canceled)

37. The lighting device of claim 1 wherein the emitted light or electromagnetic radiation comprises eight unique spectral components, optionally defined by peak wavelength and deviation from peak wavelength at half-maximum and comprising: 440±459±5 nm; 473±5 nm; 499±5 nm; 524±5 nm; 567±5 nm; 592±8 nm; and 632±8 nm.

38. The lighting device of claim 1 wherein the one or more emitters comprises a plurality of emitters, optionally wherein each of the plurality of emitters emits light or electromagnetic radiation at one or more bandwidth between 420 and 650 nm or between 300 and 780 nm.

39. The lighting device of claim 38 wherein the plurality of emitters comprise distinctly controllable emitters emitting light or electromagnetic radiation at the following spectral components comprising: 420 to 470 nm; 460 to 510 nm; 500 to 550 nm; 540 to 600 nm; and 580 to 650 nm.

40-59. (canceled)
